(19) United States
(12) Patent Application Publication
(10) Pub. No.: US 2026/0306775 A1
(43) Pub. Date: Oct. 8, 2026

(54) PATIENT INTERFACE INCLUDING FLOW GENERATOR

(71) Applicant: ResMed Pty Ltd, Bella Vista, New South Wales (AU)

(72) Inventors: Matthew Robin WELLS, Sydney (AU); Sebastien DEUBEL, Sydney (AU)

(21) Appl. No.: 19/480,734

(22) PCT Filed: May 2, 2024
(86) PCT No.: PCT/AU2024/050419

§ 371 Date: Oct. 31, 2025

Related U.S. Application Data
(60) Provisional application No. 63/463,729, filed on May 3, 2023.

(51) Int. Cl. A61M 16/06; A61M 16/00; A61M 16/08; A61M 16/16

(52) U.S. Cl. CPC A61M 16/0622; A61M 16/0069; A61M 16/024; A61M 16/0633; A61M 16/0672; A61M 16/0683; A61M 16/0875; A61M 16/16; A61M 2205/0216

(57) ABSTRACT

A patient interface for treating a patient with a respiratory disorder includes an RPT device including an electric blower configured to generate pressurized breathable air, a user interface structure configured to engage against the patient's face, the user interface structure at least partially forming a plenum chamber configured to receive the pressurized breathable air, a head-mounted interface configured to substantially cover the eyes of the patient, a flow generator casing that at least partly encloses the head-mounted interface and the electric blower and is connected to the plenum chamber, the flow generator casing including at least one air opening to receive ambient air for delivery to the RPT device, and a positioning and stabilising structure configured to maintain the user interface structure in a therapeutically effective position.

### 1 CROSS-REFERENCE TO RELATED APPLICATIONS

This application claims priority to U.S. Provisional Application No. 63/463,729, filed May 3, 2023, which is incorporated herein by reference in its entirety.

Also, PCT Application No. PCT/AU2022/051321, filed Nov. 4, 2022, is incorporated herein by reference in its entirety.

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

### 2 BACKGROUND OF THE TECHNOLOGY

### 2.1 Field of the Technology

The present technology relates to one or more of the screening, diagnosis, monitoring, treatment, prevention and amelioration of respiratory-related disorders. The present technology also relates to medical devices or apparatus, and their use.

Also, the present technology relates generally to head mounted displays, positioning and stabilising structures, user interfacing structures, and other components for use in head mounted displays, associated head-mounted display assemblies and systems including a display unit and positioning and stabilising structure, interfacing structures and or components, and methods. The present technology finds particular application in the use of virtual reality head mounted displays and is herein described in that context. However, it is to be appreciated that the present technology may have broader application and may be used in other head-mounted display arrangements including augmented reality displays.

### 2.2 Description of the Related Art

### 2.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g., apneas, hypopneas, and hyperpneas.

Examples of respiratory disorders include Obstructive Sleep Apnea (OSA), Cheyne-Stokes Respiration (CSR), respiratory insufficiency, Obesity Hypoventilation Syndrome (OHS), Chronic Obstructive Pulmonary Disease (COPD), Neuromuscular Disease (NMD) and Chest wall disorders.

Obstructive Sleep Apnea (OSA), a form of Sleep Disordered Breathing (SDB), is characterised by events including occlusion or obstruction of the upper air passage during sleep. It results from a combination of an abnormally small upper airway and the normal loss of muscle tone in the region of the tongue, soft palate and posterior oropharyngeal wall during sleep. The condition causes the affected patient to stop breathing for periods typically of 30 to 120 seconds in duration, sometimes 200 to 300 times per night. It often causes excessive daytime somnolence, and it may cause cardiovascular disease and brain damage. The syndrome is a common disorder, particularly in middle aged overweight males, although a person affected may have no awareness of the problem. See U.S. Pat. No. 4,944,310 (Sullivan).

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterised by repetitive de-oxygenation and re-oxygenation of the arterial blood. It is possible that CSR is harmful because of the repetitive hypoxia. In some patients CSR is associated with repetitive arousal from sleep, which causes severe sleep disruption, increased sympathetic activity, and increased afterload. See U.S. Pat. No. 6,532,959 (Berthon-Jones).

Respiratory failure is an umbrella term for respiratory disorders in which the lungs are unable to inspire sufficient oxygen or exhale sufficient CO2 to meet the patient's needs. Respiratory failure may encompass some or all of the following disorders.

A patient with respiratory insufficiency (a form of respiratory failure) may experience abnormal shortness of breath on exercise.

Obesity Hypoventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive: (i) Rapidly progressive disorders: Characterised by muscle impairment that worsens over months and results in death within a few years (e.g. Amyotrophic lateral sclerosis (ALS) and Duchenne muscular dystrophy (DMD) in teenagers); (ii) Variable or slowly progressive disorders: Characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

A range of therapies have been used to treat or ameliorate such conditions. Furthermore, otherwise healthy individuals may take advantage of such therapies to prevent respiratory disorders from arising. However, these have a number of shortcomings.

### 2.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 2.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube or endotracheal tube. In some forms, the comfort and effectiveness of these therapies may be improved.

### 2.2.2.2 Flow Therapies

Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure. In other cases, the interface to the patient's airways is ‘open’ (unsealed) and the respiratory therapy may only supplement the patient's own spontaneous breathing with a flow of conditioned or enriched gas. In one example, High Flow therapy (HFT) is the provision of a continuous, heated, humidified flow of air to an entrance to the airway through an unsealed or open patient interface at a “treatment flow rate” that may be held approximately constant throughout the respiratory cycle. The treatment flow rate is nominally set to exceed the patient's peak inspiratory flow rate. HFT has been used to treat OSA, CSR, respiratory failure, COPD, and other respiratory disorders. One mechanism of action is that the high flow rate of air at the airway entrance improves ventilation efficiency by flushing, or washing out, expired CO2 from the patient's anatomical deadspace. Hence, HFT is thus sometimes referred to as a deadspace therapy (DST). Other benefits may include the elevated warmth and humidification (possibly of benefit in secretion management) and the potential for modest elevation of airway pressures. As an alternative to constant flow rate, the treatment flow rate may follow a profile that varies over the respiratory cycle.

Another form of flow therapy is long-term oxygen therapy (LTOT) or supplemental oxygen therapy. Doctors may prescribe a continuous flow of oxygen enriched air at a specified oxygen concentration (from 21%, the oxygen fraction in ambient air, to 100%) at a specified flow rate (e.g., 1 litre per minute (LPM), 2 LPM, 3 LPM, etc.) to be delivered to the patient's airway.

### 2.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

### 2.2.3.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the patient interface may not include a seal sufficient to facilitate delivery to the airways of a supply of gas at a positive pressure of about 10 cmH₂O. For flow therapies such as nasal HFT, the patient interface is configured to insufflate the nares but specifically to avoid a complete seal. One example of such a patient interface is a nasal cannula.

Certain other mask systems may be functionally unsuitable for the present field. For example, purely ornamental masks may be unable to maintain a suitable pressure. Mask systems used for underwater swimming or diving may be configured to guard against ingress of water from an external higher pressure, but not to maintain air internally at a higher pressure than ambient.

Certain masks may be clinically unfavourable for the present technology e.g., if they block airflow via the nose and only allow it via the mouth.

Certain masks may be uncomfortable or impractical for the present technology if they require a patient to insert a portion of a mask structure in their mouth to create and maintain a seal via their lips.

Certain masks may be impractical for use while sleeping, e.g., for sleeping while lying on one's side in bed with a head on a pillow.

The design of a patient interface presents a number of challenges. The face has a complex three-dimensional shape. The size and shape of noses and heads varies considerably between individuals. Since the head includes bone, cartilage and soft tissue, different regions of the face respond differently to mechanical forces. The jaw or mandible may move relative to other bones of the skull. The whole head may move during the course of a period of respiratory therapy.

As a consequence of these challenges, some masks suffer from being one or more of obtrusive, aesthetically undesirable, costly, poorly fitting, difficult to use, and uncomfortable especially when worn for long periods of time or when a patient is unfamiliar with a system. Wrongly sized masks can give rise to reduced compliance, reduced comfort and poorer patient outcomes. Masks designed solely for aviators, masks designed as part of personal protection equipment (e.g., filter masks), SCUBA masks, or for the administration of anaesthetics may be tolerable for their original application, but nevertheless such masks may be undesirably uncomfortable to be worn for extended periods of time, e.g., several hours. This discomfort may lead to a reduction in patient compliance with therapy. This is even more so if the mask is to be worn during sleep.

CPAP therapy is highly effective to treat certain respiratory disorders, provided patients comply with therapy. If a mask is uncomfortable, or difficult to use a patient may not comply with therapy. Since it is often recommended that a patient regularly wash their mask, if a mask is difficult to clean (e.g., difficult to assemble or disassemble), patients may not clean their mask, and this may impact on patient compliance.

While a mask for other applications (e.g., aviators) may not be suitable for use in treating sleep disordered breathing, a mask designed for use in treating sleep disordered breathing may be suitable for other applications.

For these reasons, patient interfaces for delivery of CPAP during sleep form a distinct field.

### 2.2.3.1.1 Seal-Forming Structure

Patient interfaces may include a seal-forming structure. Since it is in direct contact with the patient's face, the shape and configuration of the seal-forming structure can have a direct impact the effectiveness and comfort of the patient interface.

A patient interface may be partly characterised according to the design intent of where the seal-forming structure is to engage with the face in use. In one form of patient interface, a seal-forming structure may comprise a first sub-portion to form a seal around the left naris and a second sub-portion to form a seal around the right naris. In one form of patient interface, a seal-forming structure may comprise a single element that surrounds both nares in use. Such single element may be designed to for example overlay an upper lip region and a nasal bridge region of a face. In one form of patient interface a seal-forming structure may comprise an element that surrounds a mouth region in use, e.g., by forming a seal on a lower lip region of a face. In one form of patient interface, a seal-forming structure may comprise a single element that surrounds both nares and a mouth region in use. These different types of patient interfaces may be known by a variety of names by their manufacturer including nasal masks, full-face masks, nasal pillows, nasal puffs and oro-nasal masks.

A seal-forming structure that may be effective in one region of a patient's face may be inappropriate in another region, e.g., because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design fit and be comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface, and is intended to seal against the patient's face when force is applied to the patient interface with the seal-forming structure in confronting engagement with the patient's face. The seal-forming structure may include an air or fluid filled cushion, or a moulded or formed surface of a resilient seal element made of an elastomer such as a rubber. With this type of seal-forming structure, if the fit is not adequate, there will be gaps between the seal-forming structure and the face, and additional force will be required to force the patient interface against the face in order to achieve a seal.

Another type of seal-forming structure incorporates a flap seal of thin material positioned about the periphery of the mask so as to provide a self-sealing action against the face of the patient when positive pressure is applied within the mask. Like the previous style of seal forming portion, if the match between the face and the mask is not good, additional force may be required to achieve a seal, or the mask may leak. Furthermore, if the shape of the seal-forming structure does not match that of the patient, it may crease or buckle in use, giving rise to leaks.

Another type of seal-forming structure may comprise a friction-fit element, e.g., for insertion into a naris, however some patients find these uncomfortable.

Another form of seal-forming structure may use adhesive to achieve a seal. Some patients may find it inconvenient to constantly apply and remove an adhesive to their face.

A range of patient interface seal-forming structure technologies are disclosed in the following patent applications, assigned to ResMed Limited: WO 1998/004310; WO 2006/074513; WO 2010/135785.

One form of nasal pillow is found in the Adam Circuit manufactured by Puritan Bennett. Another nasal pillow, or nasal puff is the subject of U.S. Pat. No. 4,782,832 (Trimble et al.), assigned to Puritan-Bennett Corporation.

ResMed Limited has manufactured the following products that incorporate nasal pillows: SWIFT™ nasal pillows mask, SWIFT™ II nasal pillows mask, SWIFT™ LT nasal pillows mask, SWIFT™ FX nasal pillows mask and MIRAGE LIBERTY™ full-face mask. The following patent applications, assigned to ResMed Limited, describe examples of nasal pillows masks: International Patent Application WO2004/073,778 (describing amongst other things aspects of the ResMed Limited SWIFT™ nasal pillows), US Patent Application 2009/0044808 (describing amongst other things aspects of the ResMed Limited SWIFT™ LT nasal pillows); International Patent Applications WO 2005/063328 and WO 2006/130903 (describing amongst other things aspects of the ResMed Limited MIRAGE LIBERTY™ full-face mask); International Patent Application WO 2009/052560 (describing amongst other things aspects of the ResMed Limited SWIFT™ FX nasal pillows).

### 2.2.3.1.2 Positioning and Stabilising

A seal-forming structure of a patient interface used for positive air pressure therapy is subject to the corresponding force of the air pressure to disrupt a seal. Thus a variety of techniques have been used to position the seal-forming structure, and to maintain it in sealing relation with the appropriate portion of the face.

One technique is the use of adhesives. See for example US Patent Application Publication No. US 2010/0000534. However, the use of adhesives may be uncomfortable for some.

Another technique is the use of one or more straps and/or stabilising harnesses. Many such harnesses suffer from being one or more of ill-fitting, bulky, uncomfortable and awkward to use.

### 2.2.3.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

Air pressure generators are known in a range of applications, e.g. industrial-scale ventilation systems. However, air pressure generators for medical applications have particular requirements not fulfilled by more generalised air pressure generators, such as the reliability, size and weight requirements of medical devices. In addition, even devices designed for medical treatment may suffer from shortcomings, pertaining to one or more of: comfort, noise, ease of use, efficacy, size, weight, manufacturability, cost, and reliability.

An example of the special requirements of certain RPT devices is acoustic noise.

Table of noise output levels of prior RPT devices
(one specimen only, measured using test method
specified in ISO 3744 in CPAP mode at 10 cmH2O).

| | A-weighted sound | Year |
|---|---|---|
| RPT Device name | pressure level dB(A) | (approx.) |
| C-Series TangoTM | 31.9 | 2007 |
| C-Series TangoTM with Humidifier | 33.1 | 2007 |
| S8 EscapeTM II | 30.5 | 2005 |
| S8 EscapeTM II with H4iTM | 31.1 | 2005 |
| Humidifier | | |
| S9 AutoSetTM | 26.5 | 2010 |
| S9 AutoSetTM with H5i Humidifier | 28.6 | 2010 |

One known RPT device used for treating sleep disordered breathing is the S9 Sleep Therapy System, manufactured by ResMed Limited. Another example of an RPT device is a ventilator. Ventilators such as the ResMed Stellar™ Series of Adult and Paediatric Ventilators may provide support for invasive and non-invasive non-dependent ventilation for a range of patients for treating a number of conditions such as but not limited to NMD, OHS and COPD.

The ResMed Elisée 150 ventilator and ResMed VS III™ ventilator may provide support for invasive and non-invasive dependent ventilation suitable for adult or paediatric patients for treating a number of conditions. These ventilators provide volumetric and barometric ventilation modes with a single or double limb circuit. RPT devices typically comprise a pressure generator, such as a motor-driven blower or a compressed gas reservoir, and are configured to supply a flow of air to the airway of a patient. In some cases, the flow of air may be supplied to the airway of the patient at positive pressure. The outlet of the RPT device is connected via an air circuit to a patient interface such as those described above.

The designer of a device may be presented with an infinite number of choices to make. Design criteria often conflict, meaning that certain design choices are far from routine or inevitable. Furthermore, the comfort and efficacy of certain aspects may be highly sensitive to small, subtle changes in one or more parameters.

### 2.2.3.3 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

### 2.2.3.4 Humidifier

Delivery of a flow of air without humidification may cause drying of airways. The use of a humidifier with an RPT device and the patient interface produces humidified gas that minimizes drying of the nasal mucosa and increases patient airway comfort. In addition, in cooler climates, warm air applied generally to the face area in and about the patient interface is more comfortable than cold air.

### 2.2.3.5 Data Management

There may be clinical reasons to obtain data to determine whether the patient prescribed with respiratory therapy has been “compliant”, e.g. that the patient has used their RPT device according to one or more “compliance rules”. One example of a compliance rule for CPAP therapy is that a patient, in order to be deemed compliant, is required to use the RPT device for at least four hours a night for at least 21 of 30 consecutive days. In order to determine a patient's compliance, a provider of the RPT device, such as a health care provider, may manually obtain data describing the patient's therapy using the RPT device, calculate the usage over a predetermined time period, and compare with the compliance rule. Once the health care provider has determined that the patient has used their RPT device according to the compliance rule, the health care provider may notify a third party that the patient is compliant.

There may be other aspects of a patient's therapy that would benefit from communication of therapy data to a third party or external system.

Existing processes to communicate and manage such data can be one or more of costly, time-consuming, and error-prone.

### 2.2.3.6 Vent Technologies

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

The vent may comprise an orifice and gas may flow through the orifice in use of the mask. Many such vents are noisy. Others may become blocked in use and thus provide insufficient washout. Some vents may be disruptive of the sleep of a bed partner 1100 of the patient 1000, e.g. through noise or focussed airflow.

ResMed Limited has developed a number of improved mask vent technologies. See International Patent Application Publication No. WO 1998/034665; International Patent Application Publication No. WO 2000/078381; U.S. Pat. No. 6,581,594; US Patent Application Publication No. US 2009/0050156; US Patent Application Publication No. 2009/0044808.

Table of noise of prior masks (ISO 17510-2:
2007, 10 cmH2O pressure at 1 m)

| | | A-weighted | A-weighted | |
|---|---|---|---|---|
| | | sound power | sound pressure | |
| | Mask | level dB(A) | dB(A) | Year |
| Mask name | type | (uncertainty) | (uncertainty) | (approx.) |
| Glue-on (*) | nasal | 50.9 | 42.9 | 1981 |
| ResCare | nasal | 31.5 | 23.5 | 1993 |
| standard (*) | | | | |
| ResMed | nasal | 29.5 | 21.5 | 1998 |
| MirageTM (*) | | | | |
| ResMed | nasal | 36 (3) | 28 (3) | 2000 |
| UltraMirageTM | | | | |
| ResMed | nasal | 32 (3) | 24 (3) | 2002 |
| Mirage | | | | |
| ActivaTM | | | | |
| ResMed | nasal | 30 (3) | 22 (3) | 2008 |
| Mirage | | | | |
| MicroTM | | | | |
| ResMed | nasal | 29 (3) | 22 (3) | 2008 |
| MirageTM | | | | |
| SoftGel | | | | |
| ResMed | nasal | 26 (3) | 18 (3) | 2010 |
| MirageTM FX | | | | |
| ResMed | nasal | 37 | 29 | 2004 |
| Mirage | pillows | | | |
| SwiftTM (*) | | | | |
| ResMed | nasal | 28 (3) | 20 (3) | 2005 |
| Mirage | pillows | | | |
| SwiftTM II | | | | |
| ResMed | nasal | 25 (3) | 17 (3) | 2008 |
| Mirage | pillows | | | |
| SwiftTM LT | | | | |
| ResMed AirFit | nasal | 21 (3) | 13 (3) | 2014 |
| P10 | pillows | | | |

(* one specimen only, measured using test method specified in ISO 3744 in CPAP mode at 10 cmH2O)

Sound pressure values of a variety of objects are listed below

| | A-weighted sound | |
|---|---|---|
| Object | pressure dB(A) | Notes |
| Vacuum cleaner: Nilfisk | 68 | ISO 3744 at 1 m |
| Walter Broadly Litter Hog: B+ | | distance |
| Grade | | |
| Conversational speech | 60 | 1 m distance |
| Average home | 50 | |
| Quiet library | 40 | |
| Quiet bedroom at night | 30 | |
| Background in TV studio | 20 | |

### 2.2.4 Screening, Diagnosis, and Monitoring Systems

Polysomnography (PSG) is a conventional system for diagnosis and monitoring of cardio-pulmonary disorders, and typically involves expert clinical staff to apply the system. PSG typically involves the placement of 15 to 20 contact sensors on a patient in order to record various bodily signals such as electroencephalography (EEG), electrocardiography (ECG), electrooculograpy (EOG), electromyography (EMG), etc. PSG for sleep disordered breathing has involved two nights of observation of a patient in a clinic, one night of pure diagnosis and a second night of titration of treatment parameters by a clinician. PSG is therefore expensive and inconvenient. In particular, it is unsuitable for home screening/diagnosis/monitoring of sleep disordered breathing.

Screening and diagnosis generally describe the identification of a condition from its signs and symptoms. Screening typically gives a true/false result indicating whether or not a patient's SDB is severe enough to warrant further investigation, while diagnosis may result in clinically actionable information. Screening and diagnosis tend to be one-off processes, whereas monitoring the progress of a condition can continue indefinitely. Some screening/diagnosis systems are suitable only for screening/diagnosis, whereas some may also be used for monitoring.

Clinical experts may be able to screen, diagnose, or monitor patients adequately based on visual observation of PSG signals. However, there are circumstances where a clinical expert may not be available, or a clinical expert may not be affordable. Different clinical experts may disagree on a patient's condition. In addition, a given clinical expert may apply a different standard at different times.

### 2.2.5 Immersive Technology

An immersive technology refers to technology that attempts to replicate or augment a physical environment through the means of a digital or virtual environment by creating a surrounding sensory feeling, thereby creating a sense of immersion.

In particular, an immersive technology provides the user visual immersion, and creates virtual objects and/or a virtual environment. The immersive technology may also provide immersion for at least one of the other five senses.

### 2.2.6 Virtual Reality

Virtual reality (VR) is a computer-generated three-dimensional image or environment that is presented to a user. In other words, the environment may be entirely virtual. Specifically, the user observes an electronic screen in order to observe virtual or computer generated images in a virtual environment. Since the created environment is entirely virtual, the user may be blocked and/or obstructed from interacting with their physical environment (e.g., they may be unable to hear and/or see the physical objects in the physical environment that they are currently located).

The electronic screen may be supported in the user's line of sight (e.g., mounted to the user's head). While observing the electronic screen, visual feedback output by the electronic screen and observed by the user may produce a virtual environment intended to simulate an actual environment. For example, the user may be able to look around (e.g., 360°) by pivoting their head or their entire body, and interact with virtual objects observable by the user through the electronic screen. This may provide the user with an immersive experience where the virtual environment provides stimuli to at least one of the user's five senses, and replaces the corresponding stimuli of the physical environment while the user uses the VR device. Typically, the stimuli relates at least to the user's sense of sight (i.e., because they are viewing an electronic screen), but other senses may also be included. The electronic screens are typically mounted to the user's head so that they may be positioned in close proximity to the user's eyes, which allows the user to easily observe the virtual environment.

The VR device may produce other forms of feedback in addition to, or aside from, visual feedback. For example, the VR device may include and/or be connected to a speaker in order to provide auditory feedback. The VR device may also include tactile feedback (e.g., in the form of haptic response), which may correspond to the visual and/or auditory feedback. This may create a more immersive virtual environment, because the user receives stimuli corresponding to more than one of the user's senses.

While using a VR device, a user may wish to limit to block ambient stimulation. For example, the user may want to avoid seeing and/or hearing the ambient environment in order to better process stimuli from the VR device in the virtual environment. Thus, VR devices may limit and/or prevent the user's eyes from receiving ambient light. In some examples, this may be done by providing a seal against the user's face. In some examples, a shield may be disposed proximate to (e.g., in contact or close contact with) the user's face, but may not seal against the user's face. In either example, ambient light may not reach the user's eyes, so that the only light observable by the user is from the electronic screen.

In other examples, the VR devices may limit and/or prevent the user's ears from hearing ambient noise. In some examples, this may be done by providing the user with headphones (e.g., noise cancelling headphones), which may output sounds from the VR device and/or limit the user from hearing noises from their physical environment. In some examples, the VR device may output sounds at a volume sufficient to limit the user from hearing ambient noise.

In any example, the user may not want to become overstimulated (e.g., by both their physical environment and the virtual environment). Therefore, blocking and/or limiting the ambient from stimulating the user assists the user in focusing on the virtual environment, without possible distractions from the ambient.

Different types of VR devices are described below. Generally, a single VR device may include at least two different classifications. For example, the VR device may be classified by its portability and by how the display unit is coupled to the rest of the interface. These classifications may be independent, so that classification in one group (e.g., the portability of the unit) does not predetermine classification into another group. There may also be additional categories to classify VR devices, which are not explicitly listed below.

### 2.2.6.1 Portability

### 2.2.6.1.1 Fixed Unit

In some forms, a VR device may be used in conjunction with a separate device, like a computer or video game console. This type of VR device may be fixed, since it cannot be used without the computer or video game console, and thus locations where it can be used are limited (e.g., by the location of the computer or video game console).

Since the VR device can be used in conjunction with the computer or video game console, the VR device may be connected to the computer or video game console. For example, an electrical cord may tether the two systems together. This may further “fix” the location of the VR device, since the user wearing the VR device cannot move further from the computer or video game console than the length of the electrical cord. In other examples, the VR device may be wirelessly connected (e.g., via Bluetooth, Wi-Fi, etc.), but may still be relatively fixed by the strength of the wireless signal.

The connection to the computer or video game console may provide control functions to the VR device. The controls may be communicated (i.e., through a wired connector or wirelessly) in order to help operate the VR device. In examples of a fixed unit VR device, these controls may be necessary in order to operate the display screen, and the VR device may not be operable without the connection to the computer or video game console.

In some forms, the computer or video game console may provide electrical power to the VR device, so that the user does not need to support a battery on their head. This may make the VR device more comfortable to wear, since the user does not need to support the weight of a battery.

The user may also receive outputs from the computer or video game console at least partially through the VR device, as opposed to through a television or monitor, which may provide the user with a more immersive experience while using the computer or video game console (e.g., playing a video game). In other words, the display output of the VR device may be substantially the same as the output from a computer monitor or television. Some controls and/or sensors necessary to output these images may be housed in the computer or video game console, which may further reduce the weight that the user is required to support on their body.

In some forms, movement sensors may be positioned remote from the VR device, and connected to the computer or video game console. For example, at least one camera may face the user in order to track movements of the user's head. The processing of the data recorded by the camera(s) may be done by the computer or video game console, before being transmitted to the VR device. While this may assist in weight reduction of the VR device, it may also further limit where the VR device can be used. In other words, the VR device must be in the sight line of the camera(s).

### 2.2.6.1.2 Portable Unit

In some forms, the VR device may be a self-contained unit, which includes a power source and sensors, so that the VR device does not need to be connected to a computer or video game console. This provides the user more freedom of use and movement. For example, the user is not limited to using the VR device near a computer or video game console, and could use the VR device outdoors, or in other environments that do not include computers or televisions.

Since the VR device is not connected to a computer or video game console in use, the VR device is required to support all necessary electronic components. This includes batteries, sensors, and processors. These components add weight to the VR device, which the user must support on their body. Appropriate weight distribution may be needed so that this added weight does not increase discomfort to a user wearing the VR device.

In some forms, the electrical components of the VR device are contained in a single housing, which may be disposed directly in front of the user's face, in use. This configuration may be referred to as a “brick.” In this configuration, the center of gravity of the VR device without the positioning and stabilizing structure is directly in front of the user's face. In order to oppose the moment created by the force of gravity, the positioning and stabilizing structure coupled to the brick configuration must provide a force directed into the user's face, for example created by tension in headgear straps. While the brick configuration may be beneficial for manufacturing (e.g., since all electrical components are in close proximity) and may allow interchangeability of positioning and stabilizing structures (e.g., because they include no electrical connections), the force necessary to maintain the position of the VR device (e.g. tensile forces in headgear) may be uncomfortable to the user. Specifically, the VR device may dig into the user's face, leading to irritation and markings on the user's skin. The combination of forces may feel like “clamping” as the user's head receives force from the display housing on their face and force from headgear on the back of their head. This may make a user less likely to wear the VR device.

As VR and other mixed reality devices may be used in a manner involving vigorous movement of the user's head and/or their entire body (for example during gaming), there may be significant forces/moments tending to disrupt the position of the device on the user's head. Simply forcing the device more tightly against the user's head to tolerate large disruptive forces may not be acceptable as it may be uncomfortable for the user or become uncomfortable after only a short period of time.

In some forms, electrical components may be spaced apart throughout the VR device, instead of entirely in front of the user's face. For example, some electrical components (e.g., the battery) may be disposed on the positioning and stabilizing structure, particularly on a posterior contacting portion. In this way, the weight of the battery (or other electrical components) may create a moment directed in the opposite direction from the moment created by the remainder of the VR device (e.g., the display). Thus, it may be sufficient for the positioning and stabilizing structure to apply a lower clamping force, which in turn creates a lower force against the user's face (e.g., fewer marks on their skin). However, cleaning and/or replacing the positioning and stabilizing structure may be more difficult in some such existing devices because of the electrical connections.

In some forms, spacing the electrical components apart may involve positioning some of the electrical components separate from the rest of the VR device. For example, a battery and/or a processor may be electrically connected, but carried separately from the rest of the VR device. Unlike in the “fixed units” described above, the battery and/or processor may be portable, along with the remainder of the VR device. For example, the battery and/or the processor may be carried on the user's belt or in the user's pocket. This may provide the benefit of reduced weight on the user's head, but would not provide a counteracting moment. The tensile force provided by the positioning and stabilizing structure may still be less than the “brick” configuration, since the total weight supported by the head is less.

### 2.2.6.2 Display Connection

### 2.2.6.2.1 Integrated Display Screen

In some forms, the display screen is an integral piece of the VR device, and generally cannot be detached or removed from the rest of the VR device.

The display screen may be fixed within a housing, and protected from damage. For example, the display screen may be completely covered by the housing, which may reduce the occurrence of scratches. Additionally, integrating display screen with the rest of the VR device eliminates the occurrence of losing the display screen.

In these forms, the display screen functions purely as an immersive technology display. The vast majority of “fixed units” will include an integrated display screen. “Portable units” may include an integrated display screen, or may include a removable display screen (described below).

### 2.2.6.2.2 Removable Display Screen

In some forms, the display screen is a separate structure that can be removed from the VR device, and used separately.

In some forms, a portable electronic device (e.g., a cell phone) may be selectively inserted into a housing of the VR device. The portable electronic device may include most or all of the sensors and/or processors, and may create a virtual environment through a downloadable app.

Portable electronic devices are generally light weight, and may not require the positioning and stabilizing structure to apply a large force to the user's head.

### 2.2.7 Augmented Reality

In some forms, augmented reality (AR) is a computer-generated three-dimensional image or environment that is presented to a user.

While similar to VR, AR differs in that the virtual environment created at least in part by the electronic screen is observed in combination with the user's physical environment. In other words, AR creates virtual objects in order to alter and/or enhance the user's physical environment with elements of a virtual environment. The result of AR is a combined environment that includes physical and virtual objects, and therefore an environment that is both physical and virtual.

For example, images created by the electronic screen may be overlayed into the user's physical environment. Only a portion of an AR combination environment presented to the user includes is virtual. Thus, the user may wish to continue to receive ambient stimulation from their physical environment while using an AR device (e.g., in order to continue to observe the physical or non-virtual component of the combination environment).

Since AR may be used with the user's physical environment, an AR device may not be electrically connected, or otherwise tethered, to a computer or video game console. Instead the AR device may include a battery, or other power source. This may provide the user with the greatest freedom of movement, so that they can explore a variety of physical environments while using the AR device.

This key difference between VR and AR may lead to different types of wearable electronic screens. As described above, a user of a VR device may wish to block ambient light, so the housing of the electronic screen may be opaque in order to limit or prevent ambient light from reaching the user. However, the user of an AR device may want to see the virtual environment blended with their actual environment. The electronic screen in an AR device may be similarly supported in front of the user's eyes, but, screens in AR devices may be transparent or translucent, and the screens may not be supported by an opaque housing (or opaque material may not substantially obstruct the user's line of sight). This may allow the user to continue receiving ambient stimulation, where the virtual environment is simultaneously present. Notwithstanding, some VR devices that do not have a transparent screen through which the user can see their real world surroundings may be configurable for AR by acquiring real-time video of the user's real-world surroundings from the user's perspective (e.g. with cameras on the display housing) and displaying it on the display screen.

Additionally, a person using an AR device may be more mobile than a person using a VR device (e.g., because an AR user can see their physical environment and/or are not tethered to a computer or video game console). Thus, a person using an AR device may wish to wear the device for an extended period of time, while also moving around (e.g., walking, running, biking, etc.). Including components, like batteries, on the AR device may make the AR device uncomfortable for the user's head and/or neck, and may discourage the user from wearing the AR device for long periods of time.

### 2.2.8 Mixed Reality

Mixed reality (MR) is similar to AR but may be more immersive because the MR device may provide the user more ways to interact with virtual objects or environment than an AR device. The virtual reality in MR may also be overlayed and/or blended with the user's physical environment. Unlike AR however, a user may be able to interact with the virtual environment akin to what occurs in VR. In other words, while AR may present only an computer generated image in the physical environment, MR may present the user with the same or similar computer generated image but allow for interaction with the image in the physical environment (e.g., using a hand to “grab” an object produced virtually). Thus, the virtual environment may further merge with a physical environment so that the combined environment better replicates an actual environment.

### 2.2.9 Head-Mounted Display Interface

A head-mounted display interface enables a user to have an immersive experience of a virtual environment and have broad application in fields such as communications, training, medical and surgical practice, engineering, and video gaming.

Different head-mounted display interfaces can each provide a different level of immersion. For example, some head-mounted display interfaces can provide the user with a total immersive experience. One example of a total immersive experience is virtual reality (VR). The head-mounted display interface can also provide partial immersion consistent with using an AR device.

VR head-mounted display interfaces typically are provided as a system that includes a display unit which is arranged to be held in an operational position in front of a user's face. The display unit typically includes a housing containing a display and a user interface structure constructed and arranged to be in opposing relation with the user's face. The user interface structure may extend about the display and define, in conjunction with the housing, a viewing opening to the display. The user interfacing structure may engage with the face and include a cushion for user comfort and/or be light sealing to block ambient light from the display. The head-mounted display system further comprises a positioning and stabilizing structure that is disposed on the user's head to maintain the display unit in position.

Other head-mounted display interfaces can provide a less than total immersive experience. In other words, the user can experience elements of their physical environment, as well as a virtual environment. Examples of a less than total immersive experience are augmented reality (AR) and mixed reality (MR).

AR and/or MR head-mounted display interfaces are also typically provided as a system that includes a display unit which is arranged to be held in an operational position in front of a user's face. Likewise, the display unit typically includes a housing containing a display and a user interface structure constructed and arranged to be in opposing relation with the user's face. The head-mounted display system of the AR and/or MR head-mounted display is also similar to VR in that it further comprises a positioning and stabilizing structure that is disposed on the user's head to maintain the display unit in position. However, AR and/or MR head-mounted displays do not include a cushion that totally seals ambient light from the display, since these less than total immersive experience require an element of the physical environment. Instead, head-mounted displays in augmented and/or mixed allow the user to see the physical environment in combination with the virtual environment.

In any types of immersive technology, it is important that the head-mounted display interface is comfortable in order to allow the user to wear the head-mounted display for extended periods of time. Additionally, it is important that the display is able to provide changing images with changing position and/or orientation of the user's head in order to create an environment, whether partially or entirely virtual, that is similar to or replicates one that is entirely physical.

### 2.2.9.1 Interfacing Structure

The head-mounted displays may include a user interfacing structure. Since it is in direct contact with the user's face, the shape and configuration of the interfacing portion can have a direct impact on the effectiveness and comfort of the display unit.

The design of a user interfacing structure presents a number of challenges. The face has a complex three-dimensional shape. The size and shape of noses and heads varies considerably between individuals. Since the head includes bone, cartilage and soft tissue, different regions of the face respond differently to mechanical forces.

One type of interfacing structure extends around the periphery of the display unit and is intended to seal against the user's face when force is applied to the user interface with the interfacing structure in confronting engagement with the user's face. The interfacing structure may include a pad made of a polyurethane (PU). With this type of interfacing structure, there may be gaps between the interfacing structure and the face, and additional force may be required to force the display unit against the face in order to achieve the desired contact.

The regions not engaged at all by the user interface may allow gaps to form between the facial interface and the user's face through which undesirable light pollution may ingress into the display unit (e.g., particularly when using virtual reality). The light pollution or “light leak” may decrease the efficacy and enjoyment of the overall immersive experience for the user. In addition, previous systems may be difficult to adjust to enable application for a wide variety of head sizes. Further still, the display unit and associated stabilizing structure may often be relatively heavy and may be difficult to clean which may thus further limit the comfort and useability of the system.

Another type of interfacing structure incorporates a flap seal of thin material positioned about a portion of the periphery of the display unit so as to provide a sealing action against the face of the user. Like the previous style of interfacing structure, if the match between the face and the interfacing structure is not good, additional force may be required to achieve a seal, or light may leak into the display unit in-use. Furthermore, if the shape of the interfacing structure does not match that of the user, it may crease or buckle in-use, giving rise to undesirable light penetration.

A user interface may be partly characterised according to the design intent of where the interfacing structure is to engage with the face in-use. Some interfacing structures may be limited to engaging with regions of the user's face that protrude beyond the arc of curvature of the face engaging surface of the interfacing structure. These regions may typically include the user's forehead and cheek bones. This may result in user discomfort at localised stress points. Other facial regions may not be engaged at all by the interfacing structure or may only be engaged in a negligible manner that may thus be insufficient to increase the translation distance of the clamping pressure. These regions may typically include the sides of the user's face, or the region adjacent and surrounding the users nose. To the extent to which there is a mismatch between the shape of the users' face and the interfacing structure, it is advantageous for the interfacing structure or a related component to be adaptable in order for an appropriate contact or other relationship to form.

### 2.2.9.2 Positioning and Stabilizing

To hold the display unit in its correct operational position, the head-mounted display system further comprises a positioning and stabilizing structure that is disposed on the user's head. These structures may be responsible for providing forces to counter gravitational forces of the head-mounted display and/or interfacing structure. In the past these structures have been formed from expandable rigid structures that are typically applied to the head under tension to maintain the display unit in its operational position. Such systems have been prone to exert a clamping pressure on the user's face which can result in user discomfort at localised stress points. Also, previous systems may be difficult to adjust to allow wide application head sizes. Further, the display unit and associated stabilizing structure are often heavy, difficult to clean which further limit the comfort and useability of the system.

Certain other head mounted display systems may be functionally unsuitable for the present field. For example, positioning and stabilizing structures designed for ornamental and visual aesthetics may not have the structural capabilities to maintain a suitable pressure around the face. For example, an excess of clamping pressure may cause discomfort to the user, or alternatively, insufficient clamping pressure on the users' face may not effectively seal the display from ambient light.

Certain other head mounted display systems may be uncomfortable or impractical for the present technology. For example, if the system is used for prolonged time periods.

As a consequence of these challenges, some head mounted displays suffer from being one or more of obtrusive, aesthetically undesirable, costly, poorly fitting, difficult to use, and uncomfortable especially when worn for long periods of time or when a user is unfamiliar with a system. Wrongly sized positioning and stabilizing structures can give rise reduced comfort and in turn, shortened periods of use.

Therefore, an interfacing portion of a user interface used for the fully immersive experience of a virtual environment are subject to forces corresponding to the movement of a user during the experience.

### 2.2.9.3 Materials

Materials used in head mounted display assemblies have included dense foams for contacting portions in the interfacing structures, rigid shells for the housings, and positioning and stabilizing structures formed from rigid plastic clamping structures. These materials have various drawbacks including not permitting the skin covered by the material to breath, being inflexible, difficult to clean and to prone trapping bacteria. As a result, products made with such material may be uncomfortable to wear for extended periods of time, causes skin irritation in some individuals and limit the application of the products.

### 3 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

One form of the present technology comprises the patient interface includes a flow generator for providing a flow of pressurized air to the patient.

Another aspect of one form of the present technology is the flow generator casing connected to a cushion of a patient interface, the flow generator casing housing a blower for providing pressurized air to the cushion.

Another aspect of one form of the present technology is the power source removably connected to the patient interface for providing electrical energy to the patient interface.

In one form, the power source is a rechargeable battery connected directly to the patient interface.

Another aspect of one form of the present technology is a patient interface includes a cushion and a blower in a blower housing connected directly to the cushion. The patient interface also includes a power source providing electrical energy to the blower.

In one form, the blower and/or the power source are worn and supported by the user in use.

Another aspect of one form of the present technology is a patient interface including a plenum chamber pressurizable to a therapeutic pressure; a seal-forming structure configured to form a seal against the patient's face; a positioning and stabilising structure configured to provide a force for maintaining the seal-forming structure in a therapeutically effective position; an RPT device connected directly to the plenum chamber, the PRT device including a blower for providing airflow at the therapeutic pressure, wherein the positioning and stabilising structure configured to support at least part of the weight of the RPT device; and an electrical power source electrically connected to the RPT device.

In some forms, a) the positioning and stabilising structure may support at least a portion of the weight of the electrical power source; b) the electrical power source is a rechargeable battery; and/or c) the patient interface is removably connected to a charger for recharging the battery.

In some forms, a) the RPT device includes a flow generator casing with a cavity housing a blower; b) the cavity is in fluid communication with the plenum chamber; and/or c) the flow generator casing extends around at least a portion of the plenum chamber.

In some forms, a) the RPT device includes a valve positioned between the blower and the plenum chamber; and/or b) the valve is a one-way valve.

Another aspect of one form of the present technology is a patient interface that is moulded or otherwise constructed with a perimeter shape which is complementary to that of an intended wearer.

Another aspect of one form of the present technology is a head-mounted display interface comprising: a user interface structure configured to contact the user's face, the user interface structure including at least one first opening configured to receive at least one of the user's eyes, and at least one second opening configured to receive at least one of the user's nares; a display unit housing connected to the user interface structure, the display unit housing including a display configured to output a computer generated image through the at least one first opening, and a blower configured to output a flow of pressurized air through the at least one second opening; and a support structure connected to the display unit housing and/or the user interface structure and configured to maintain the user interface structure in a desired position on the user's face.

In some forms, the blower is constructed and arranged to be able to provide a supply of air at a positive pressure of at least 4 cmH2O with respect to ambient.

In some forms, the head-mounted display interface further includes an audio system configured to output sound to a user.

Another aspect of the present technology is directed to a patient interface for treating a patient with a respiratory disorder, comprising a respiratory pressure therapy (RPT) device including an electric blower configured to generate pressurized breathable air; a seal-forming structure configured to form a seal against the patient's face, the seal-forming structure at least partially defining a plenum chamber configured to receive the pressurized air; a flow generator casing that at least partly encloses the electric blower and is connected to the plenum chamber, the casing including at least one air opening to receive ambient air for delivery to the RPT device; and a positioning and stabilising structure configured to maintain the seal-forming structure and the blower in a therapeutically effective position.

In some forms the patient interface may include at least one of the following:
the patient interface includes no tube or forehead support that is configured to extend between the patient's eyes,
the positioning and stabilising structure is connected to the casing and/or the plenum chamber,
the casing includes a rear case and a front case that define a cavity in which the blower is fully enclosed,
the casing has a curvature configured to wrap about the patient's face,
the curvature of the front case and the curvature of the rear case are complimentary,
the casing includes a central section and two side sections, the central portion housing the blower, the central section and the side sections providing a continuous curvature,
the central section of the front case is detachable from the side sections, or wherein
the central section of the front case is formed in one piece with the side sections of the front case,
the front case is removably attached to the rear case to expose the blower within the cavity and one or more electrical components within the cavity,
the one or more electrical components includes a pressure sensor,
the rear case is at least partly covered with a textile,
the blower has a substantially cylindrical shape and is arranged laterally within the cavity anterior to the rear case,
a longitudinal axis of the blower runs perpendicular to an inlet of the casing that leads to the plenum chamber,
a suspension to support the blower within the cavity,
the suspension includes at least a first opening to allow flow of ambient air into the blower, and a second opening to receive the air after pressurization,
a manifold within the cavity and configured to guide the pressurized air from the blower towards the plenum chamber,
the manifold redirects the pressurized air from one direction to another direction,
the suspension includes an opening configured to direct pressurized air generated by the blower to the manifold,
the rear case includes a central groove or recess that receives and supports the blower, the central groove or recess extends laterally and is semi-cylindrical in shape,
the plenum chamber includes an anterior surface with a concave section that receives a convex exterior part of the casing or rear case that houses the blower,
the casing or rear case includes an inlet tube positioned above the convex exterior part of the casing or rear case, and the plenum chamber includes an inlet opening removably attached to the inlet tube and positioned above the concave portion of the plenum chamber, an expiratory activated valve to direct pressurized gas from the cavity to the plenum chamber during inhalation and/or from the plenum chamber to an exhaust channel of the casing during exhalation,
the valve includes a one-way duck bill valve to direct incoming pressurized gas to the plenum chamber,
the EAV includes a membrane movable during exhalation to guide exhaled air along outside of the duckbill valve, such that in use the exhaled air passes through an exhaust channel, and to ambient via a gas washout vent,
exhaust channel directs exhaled gas to a gas washout vent having one or more holes leading to ambient,
the valve is positioned within an inlet of the casing leading to the plenum chamber,
at least one muffler positioned at at least one end of the blower,
at least one outlet vent configured to allow exhaled gas to exhaust to ambient,
the outlet vent is provided as part of the front case and/or rear case,
the blower includes at least a pair of impellers coupled to a common shaft and arranged in parallel,
each of the impellers is a double-sided impeller,
an elastomeric bearing configured to limit vibrations of the blower,
the seal-forming portion includes a pillows mask, a full face mask or a nasal mask,
the seal-forming portion includes a first portion to surround the patient's mouth and a second portion to seal with a lower surface of and/or underneath the patient's nose,
the second portion includes a pair of openings to align with the patient's nares,
the seal-forming portion does not extend over the patient's nasal ridge,
an electrical connector for connection to a power cord to power the blower with a power source, and/or a battery to supply power to the blower,
a battery connected to a strap of the positioning and stabilising structure, and an electrical conductor formed as part of the strap and connected to the blower, and/or
a power cord connected to a strap of the positioning and stabilising structure or to the casing, the power cord being configured to conduct electricity form an AC outlet or a remote battery.

The present technology may also be directed toward providing interfacing structures used in the supporting, cushioning, stabilizing, positioning, and/or sealing a head-mounted display in opposing relation with the user's face.

Another aspect relates to apparatuses used in the supporting, cushioning, stabilizing, positioning, and/or sealing a head-mounted display in opposing relation with the user's face.

Another aspect relates to methods used in supporting, cushioning, stabilizing, positioning, and/or sealing a head-mounted display in opposing relation with the user's face.

An aspect of one form of the present technology relates to a patient interface combining the features of AR/VR and respiratory therapy.

An aspect of one form of the present technology relates to a patient interface for treating a patient with a respiratory disorder includes an RPT device including an electric blower configured to generate pressurized breathable air, a user interface structure configured to engage against the patient's face, the user interface structure at least partially forming a plenum chamber configured to receive the pressurized breathable air, a head-mounted interface configured to substantially cover the eyes of the patient, a flow generator casing that at least partly encloses the head-mounted interface and the electric blower and is connected to the plenum chamber, the flow generator casing including at least one air opening to receive ambient air for delivery to the RPT device, and a positioning and stabilising structure configured to maintain the user interface structure in a therapeutically effective position.

In an example, the head-mounted interface is the form of a head-mounted display interface configured to generate a computer-generated image to the patient. In an alternative example, the head-mounted interface may not include any display or screen and may not generate any computer-generated image.

In an example, the user interface structure includes a stability cushion component and a therapy cushion component that is separate and distinct from the stability cushion component.

In an example, the stability cushion component is configured to extend about a periphery of the patient's eyes and form a viewing opening to the head-mounted interface.

In an example, the therapy cushion component is configured to form the plenum chamber and form a seal with the patient's nose and/or mouth.

In an example, the stability cushion component is configured to removably and interchangeably couple with the flow generator casing, and the therapy cushion component is configured to removably and interchangeably couple with the stability cushion component and/or the flow generator casing.

In an example, the stability cushion component is configured to engage the patient's face to provide substantially complete sealing around the patient's eyes.

In an example, the stability cushion component includes a chassis and a cushion configured to engage with the patient's face, and the chassis is configured and arranged to repeatably engage with and disengage from the flow generator casing.

In an example, the chassis is relatively harder than the cushion.

In an example, the cushion includes a face contacting portion configured to engage with the patient's face and a non-face contacting portion configured to connect the cushion to the chassis.

In an example, the face contacting portion comprises a foam cushion including a face engaging surface configured to contact the patient's face around a periphery of the user's eyes.

In an example, the face contacting portion comprises a silicone membrane including a face engaging surface configured to contact the patient's face around a periphery of the user's eyes.

In an example, the non-face contacting portion includes a side wall provided to the chassis and a ledge configured to project away from the side wall in a cantilever manner, the non-face contacting portion configured to provide a resilient, spring-like support to the face contacting portion.

In an example, the chassis of the stability cushion component provides an interface with the therapy cushion component.

In an example, the interface comprises a manifold configured to form an airflow path to convey the pressurized breathable air from the blower to the therapy cushion component.

In an example, the therapy cushion component is configured to removably and interchangeably couple with the flow generator casing.

In an example, the therapy cushion component includes a front portion and a cushion configured to form a seal with the patient's nose and/or mouth.

In an example, the front portion is relatively harder than the cushion.

In an example, the cushion is a nasal cushion including a silicone membrane configured to form a seal with the patient's nose.

In an example, the stability cushion component is provided in one size and/or type and the therapy cushion component is provided in multiple sizes and/or types.

In an example, the patient interface further includes a manifold configured to form an airflow path to convey the pressurized breathable air from the blower to the therapy cushion component.

In an example, the manifold and the therapy cushion component cooperate to house an expiratory activated valve (EAV) to direct the pressurized breathable air from the blower to the therapy cushion component during inhalation and direct the pressurized breathable air from the therapy cushion component to an exhaust channel during exhalation.

In an example, the blower includes at least a pair of impellers coupled to a common shaft and arranged in parallel.

In an example, the patient interface further includes a pair of batteries supported by the flow generator casing on respective sides of the blower.

In an example, the positioning and stabilising structure is connected to at least a portion of the flow generator casing.

An aspect of one form of the present technology relates to a user interface structure for a patient interface configured to treat a patient with a respiratory disorder, the user interface structure including a stability cushion component and a therapy cushion component that is separate and distinct from the stability cushion component.

In an example, the stability cushion component is configured to extend about a periphery of the patient's eyes.

In an example, the therapy cushion component is configured to form a plenum chamber and form a seal with the patient's nose and/or mouth.

An aspect of one form of the present technology relates to a stability cushion component for a patient interface configured to treat a patient with a respiratory disorder, the stability cushion component configured to extend about a periphery of the patient's eyes.

An aspect of one form of the present technology relates to a therapy cushion component for a patient interface configured to treat a patient with a respiratory disorder, the therapy cushion component is configured to form a plenum chamber and form a seal with the patient's nose and/or mouth.

An aspect of one form of the present technology relates to full-face patient interface for respiratory therapy including a seal-forming structure with a two-part cushion construction.

In an example, the seal-forming structure includes a nasal cushion component and a mouth cushion component that is separate and distinct from the nasal cushion component.

In an example, the nasal cushion component is configured to removably and interchangeably couple with the casing, and the mouth cushion component is configured to removably and interchangeably couple with the casing independent from the nasal cushion component.

An aspect of one form of the present technology is a method of manufacturing apparatus.

An aspect of certain forms of the present technology is a medical device that is easy to use, e.g. by a person who does not have medical training, by a person who has limited dexterity, vision or by a person with limited experience in using this type of medical device.

An aspect of one form of the present technology is a portable RPT device that may be carried by a person, e.g., around the home of the person.

An aspect of one form of the present technology is a patient interface that may be washed in a home of a patient, e.g., in soapy water, without requiring specialised cleaning equipment. An aspect of one form of the present technology is a humidifier tank that may be washed in a home of a patient, e.g., in soapy water, without requiring specialised cleaning equipment.

The methods, systems, devices and apparatus described may be implemented so as to improve the functionality of a processor, such as a processor of a specific purpose computer, respiratory monitor and/or a respiratory therapy apparatus. Moreover, the described methods, systems, devices and apparatus can provide improvements in the technological field of automated management, monitoring and/or treatment of respiratory conditions, including, for example, sleep disordered breathing.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 4 BRIEF DESCRIPTION OF THE DRAWINGS

The present technology is illustrated by way of example, and not by way of limitation, in the figures of the accompanying drawings, in which like reference numerals refer to similar elements including:

### 4.1 Respiratory Therapy Systems

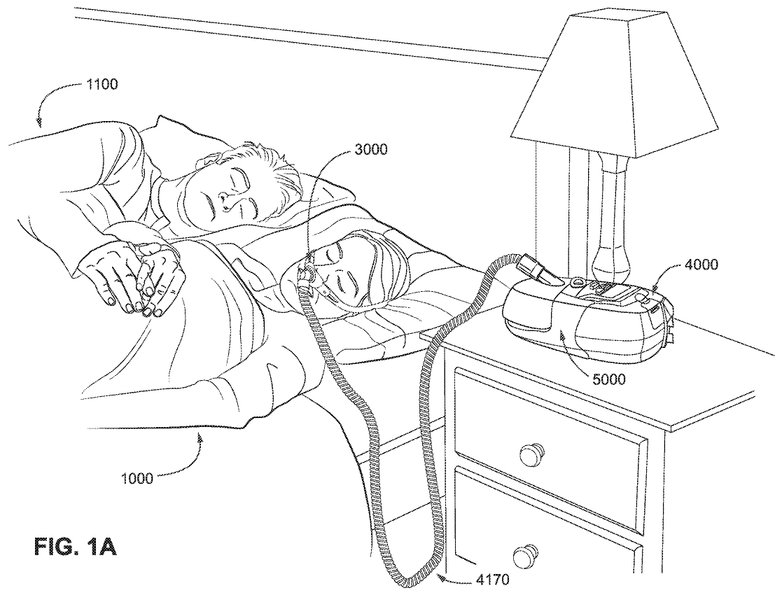

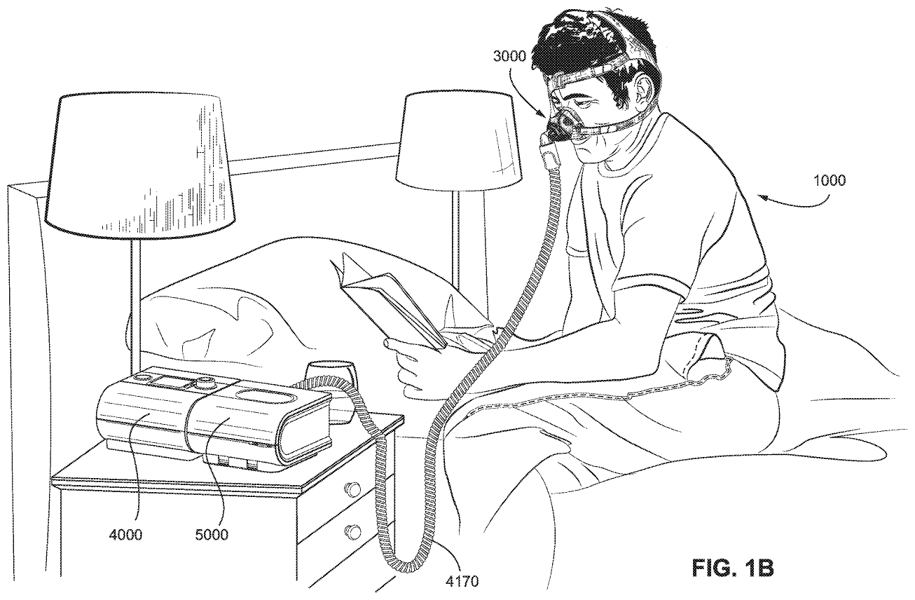

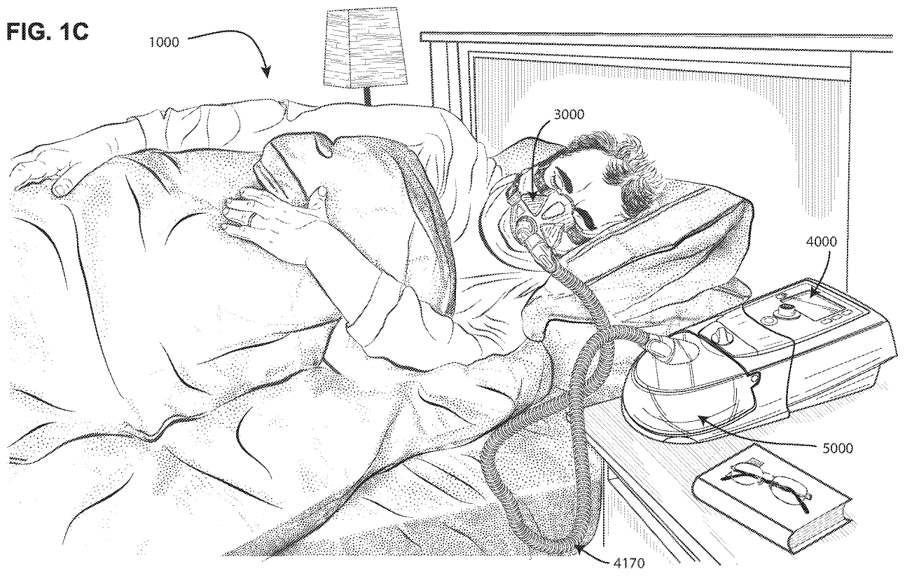

FIG. 1A shows a system including a patient 1000 wearing a patient interface 3000, in the form of nasal pillows, receiving a supply of air at positive pressure from an RPT device 4000. Air from the RPT device 4000 is humidified in a humidifier 5000, and passes along an air circuit 4170 to the patient 1000. A bed partner 1100 is also shown. The patient is sleeping in a supine sleeping position.

FIG. 1B shows a system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 1C shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 4.2 Respiratory System and Facial Anatomy

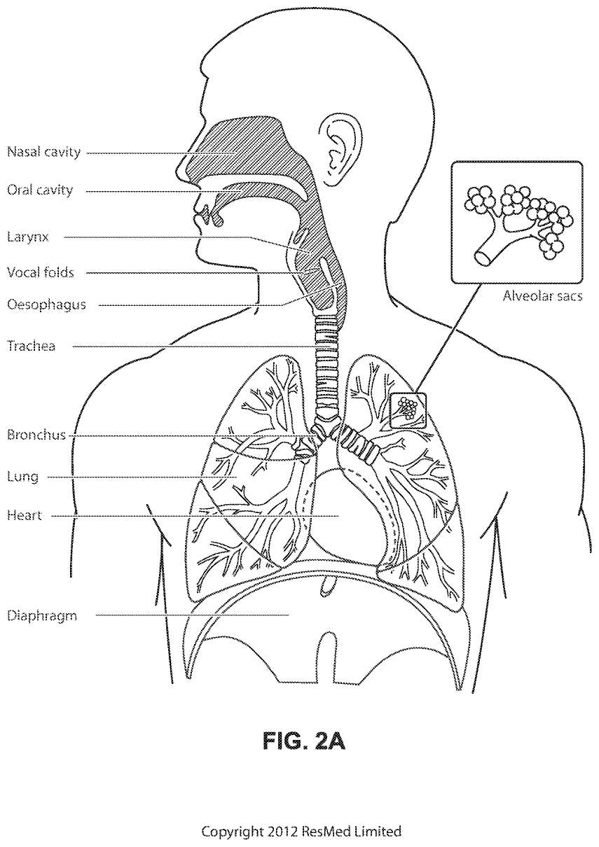

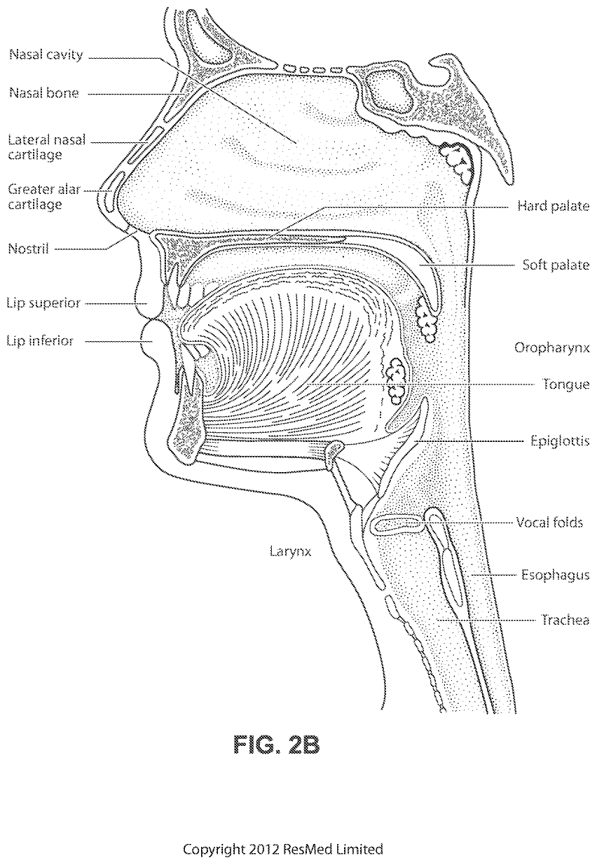

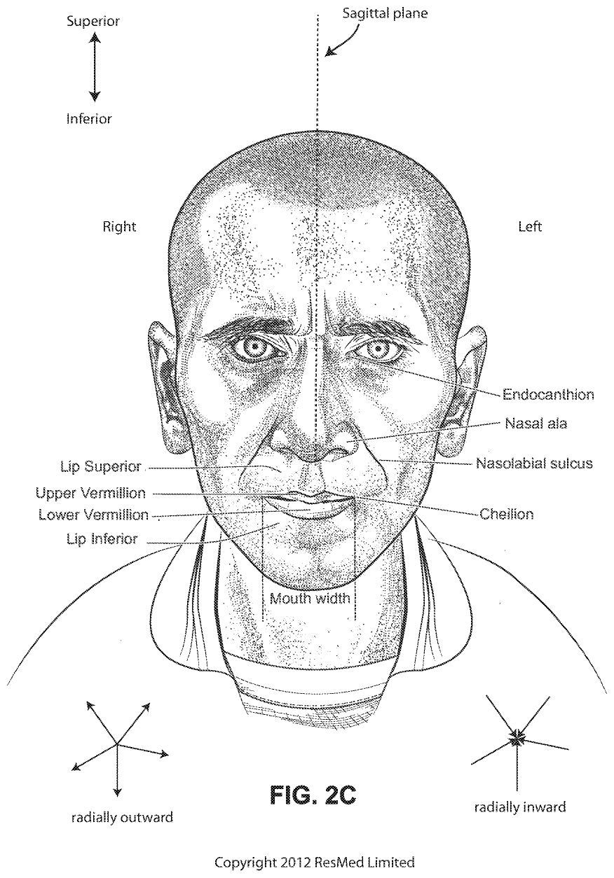

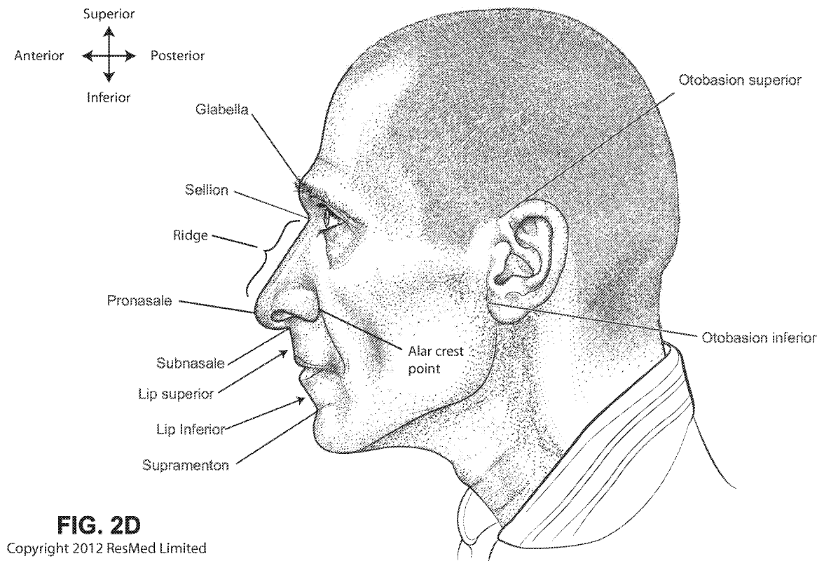

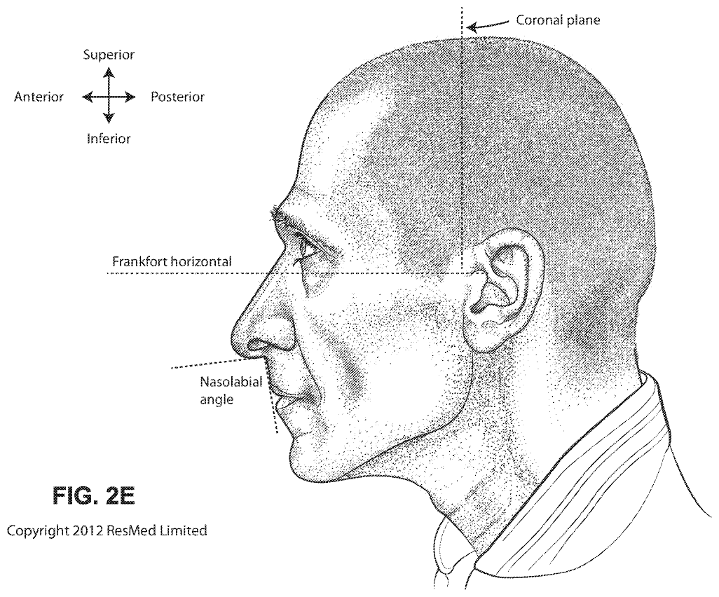

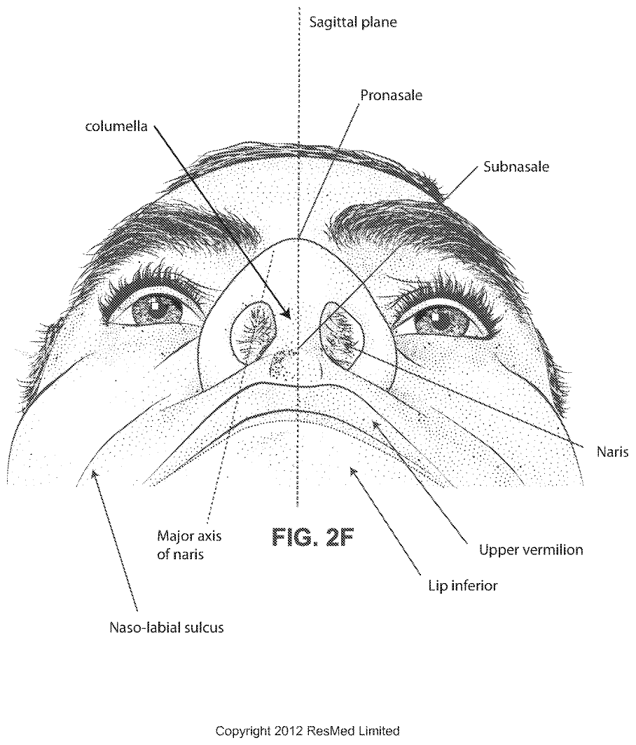

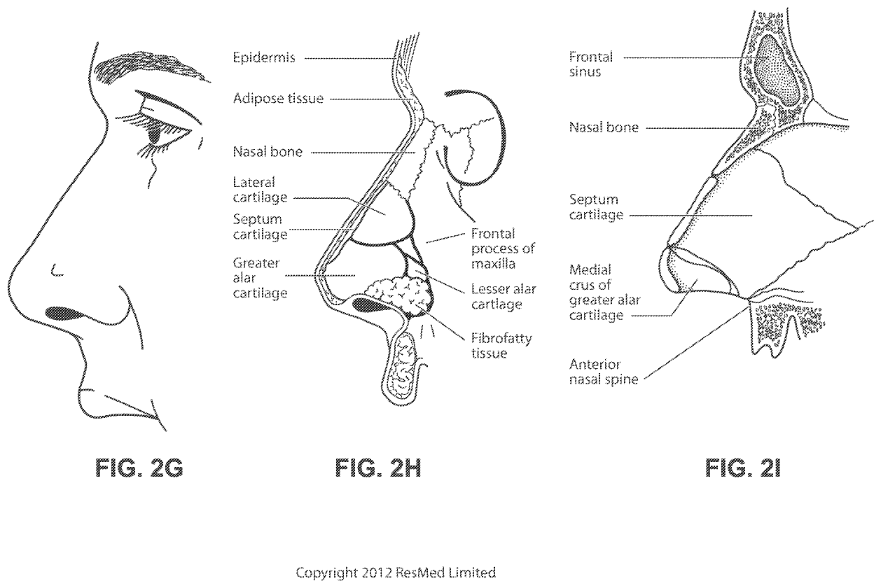

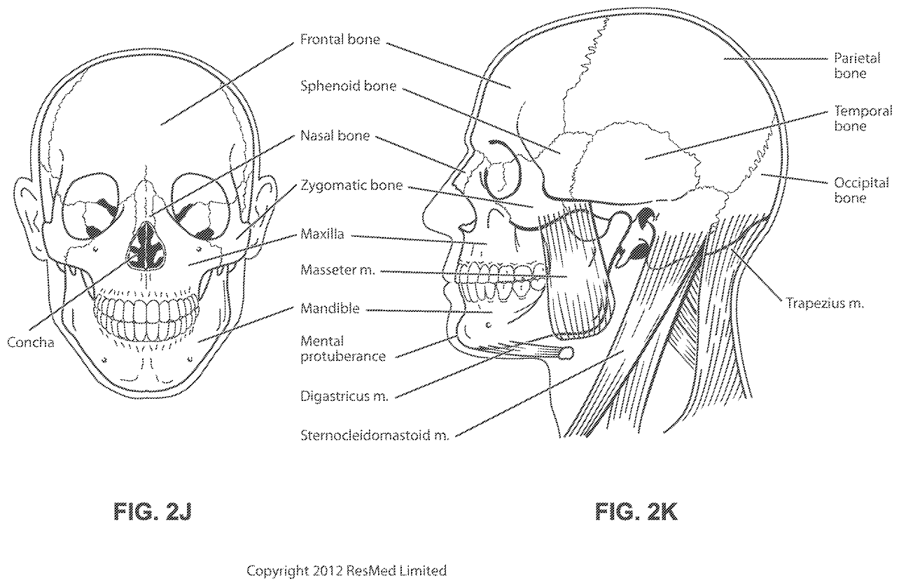

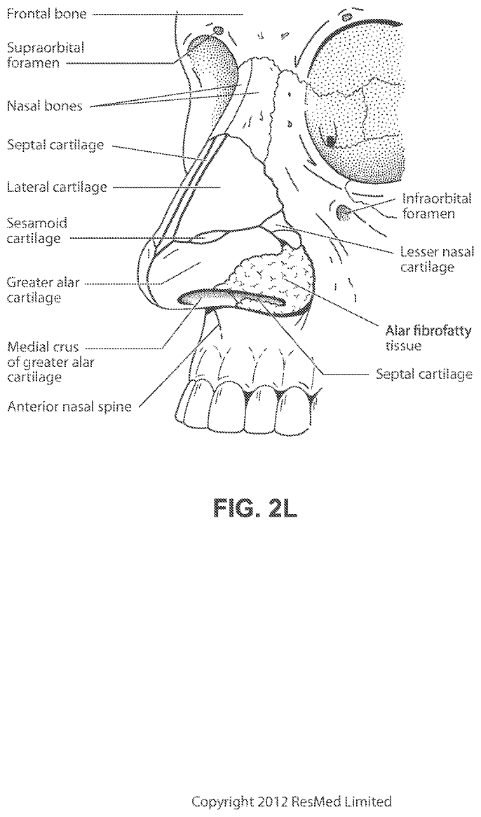

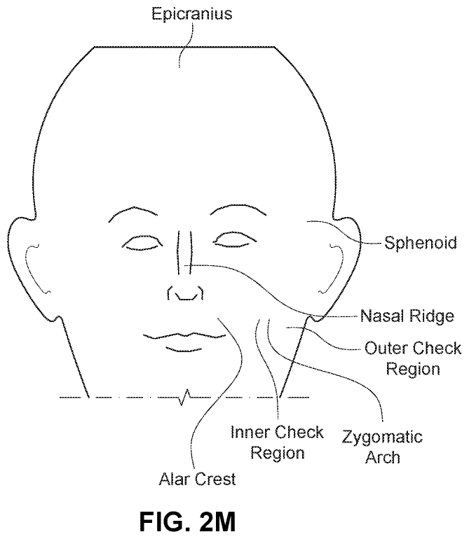

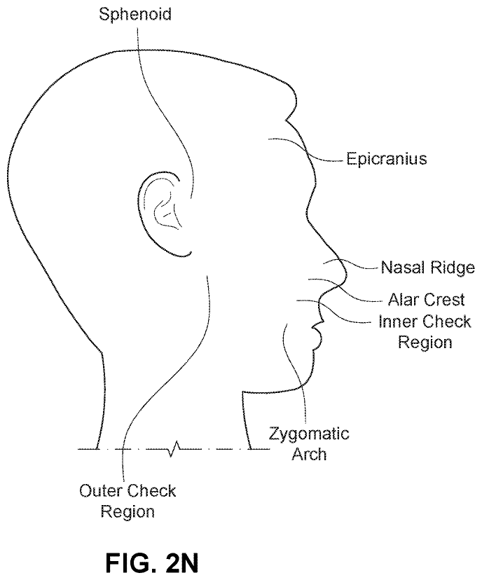

FIG. 2A shows an overview of a human respiratory system including the nasal and oral cavities, the larynx, vocal folds, oesophagus, trachea, bronchus, lung, alveolar sacs, heart and diaphragm.

FIG. 2B shows a view of a human upper airway including the nasal cavity, nasal bone, lateral nasal cartilage, greater alar cartilage, nostril, lip superior, lip inferior, larynx, hard palate, soft palate, oropharynx, tongue, epiglottis, vocal folds, oesophagus and trachea.

FIG. 2C is a front view of a face with several features of surface anatomy identified including the lip superior, upper vermilion, lower vermilion, lip inferior, mouth width, endocanthion, a nasal ala, nasolabial sulcus and cheilion. Also indicated are the directions superior, inferior, radially inward and radially outward.

FIG. 2D is a side view of a head with several features of surface anatomy identified including glabella, sellion, pronasale, subnasale, lip superior, lip inferior, supramenton, nasal ridge, alar crest point, otobasion superior and otobasion inferior. Also indicated are the directions superior & inferior, and anterior & posterior.

FIG. 2E is a further side view of a head. The approximate locations of the Frankfort horizontal and nasolabial angle are indicated. The coronal plane is also indicated.

FIG. 2F shows a base view of a nose with several features identified including naso-labial sulcus, lip inferior, upper Vermilion, naris, subnasale, columella, pronasale, the major axis of a naris and the midsagittal plane.

FIG. 2G shows a side view of the superficial features of a nose.

FIG. 2H shows subcutaneal structures of the nose, including lateral cartilage, septum cartilage, greater alar cartilage, lesser alar cartilage, sesamoid cartilage, nasal bone, epidermis, adipose tissue, frontal process of the maxilla and fibrofatty tissue.

FIG. 2I shows a medial dissection of a nose, approximately several millimeters from the midsagittal plane, amongst other things showing the septum cartilage and medial crus of greater alar cartilage.

FIG. 2J shows a front view of the bones of a skull including the frontal, nasal and zygomatic bones. Nasal concha are indicated, as are the maxilla, and mandible.

FIG. 2K shows a lateral view of a skull with the outline of the surface of a head, as well as several muscles. The following bones are shown: frontal, sphenoid, nasal, zygomatic, maxilla, mandible, parietal, temporal and occipital. The mental protuberance is indicated. The following muscles are shown: digastricus, masseter, sternocleidomastoid and trapezius.

FIG. 2L shows an anterolateral view of a nose.

FIG. 2M shows another a view of the face with several features of surface anatomy identified including the epicranius, the sphenoid, the nasal ridge, the outer and inner cheek regions, the zygomatic arch, and the alar crest.

FIG. 2N shows a side view of the face with several features of surface anatomy identified including the epicranius, the sphenoid, the nasal ridge, the outer and inner cheek regions, the zygomatic arch, and the alar crest.

### 4.3 Patient Interface

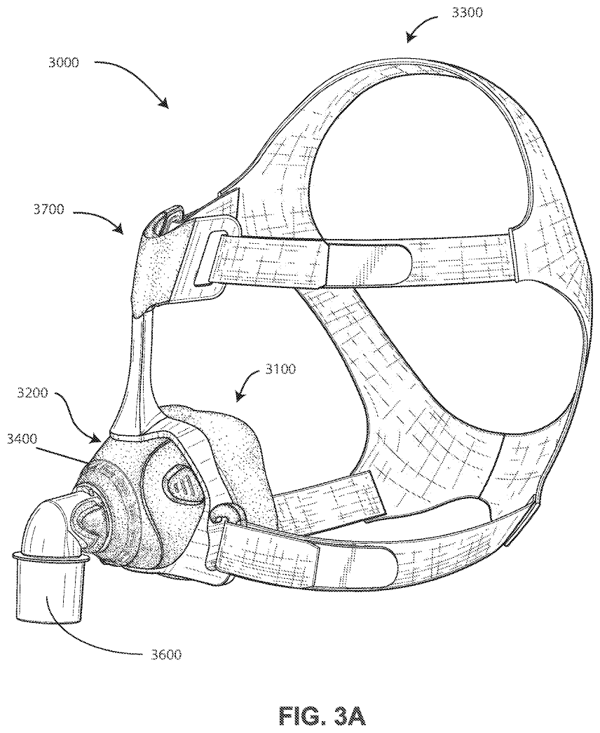

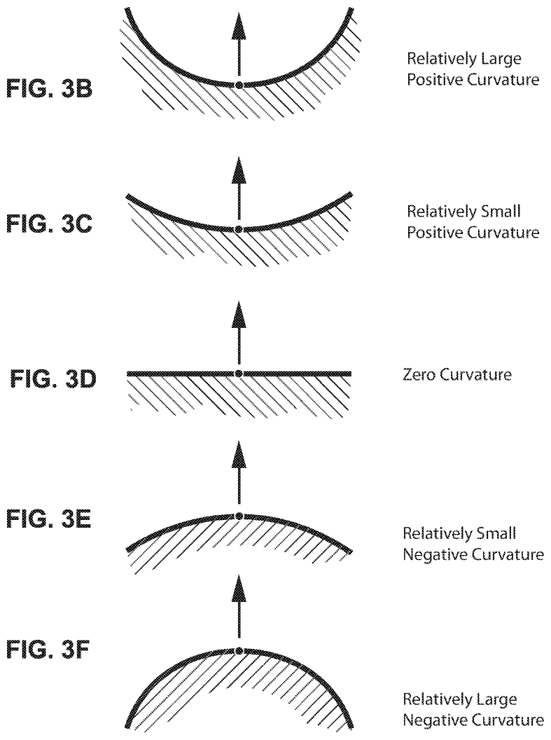

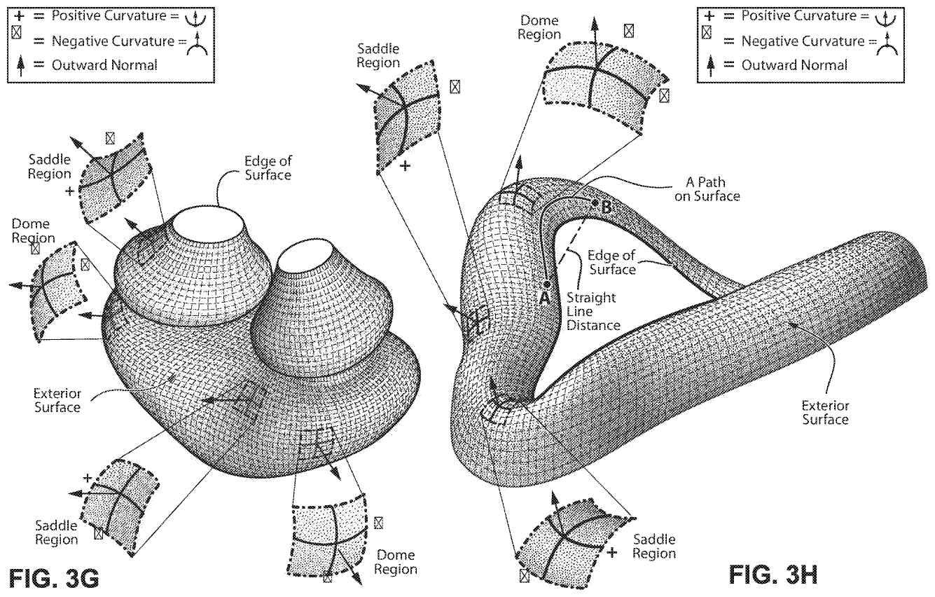

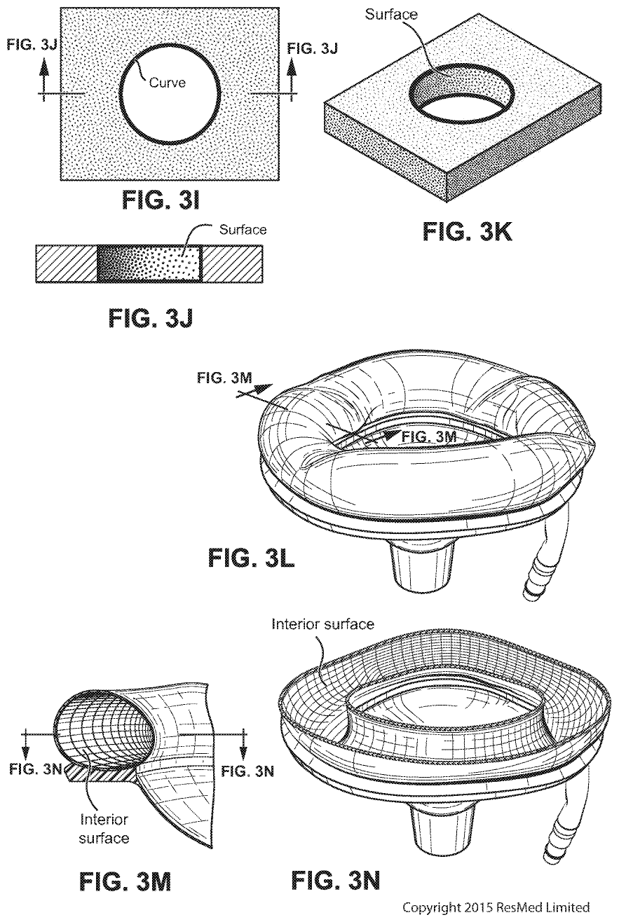

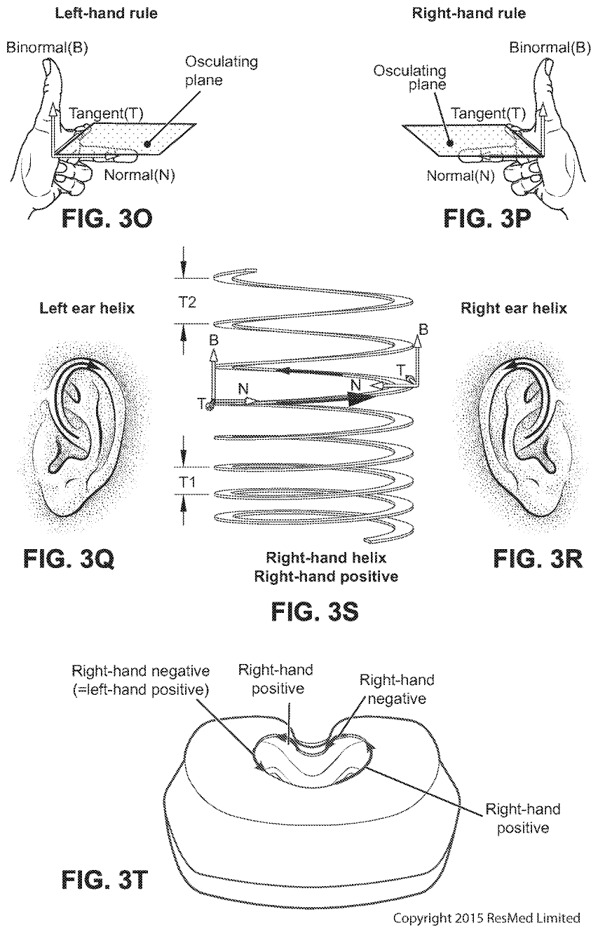

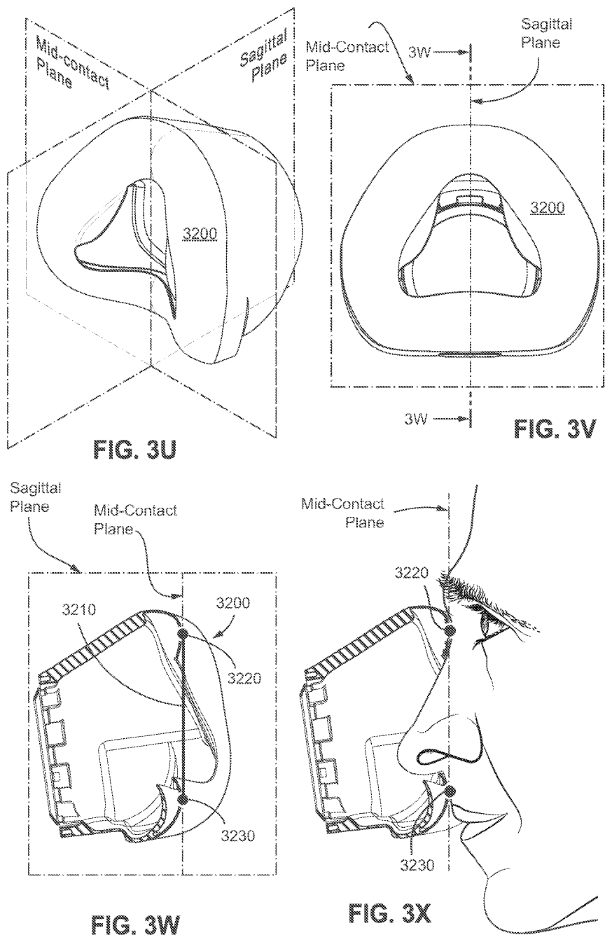

FIG. 3A shows a patient interface in the form of a nasal mask in accordance with one form of the present technology.

FIG. 3B shows a schematic of a cross-section through a structure at a point. An outward normal at the point is indicated. The curvature at the point has a positive sign, and a relatively large magnitude when compared to the magnitude of the curvature shown in FIG. 3C.

FIG. 3C shows a schematic of a cross-section through a structure at a point. An outward normal at the point is indicated. The curvature at the point has a positive sign, and a relatively small magnitude when compared to the magnitude of the curvature shown in FIG. 3B.

FIG. 3D shows a schematic of a cross-section through a structure at a point. An outward normal at the point is indicated. The curvature at the point has a value of zero.

FIG. 3E shows a schematic of a cross-section through a structure at a point. An outward normal at the point is indicated. The curvature at the point has a negative sign, and a relatively small magnitude when compared to the magnitude of the curvature shown in FIG. 3F.

FIG. 3F shows a schematic of a cross-section through a structure at a point. An outward normal at the point is indicated. The curvature at the point has a negative sign, and a relatively large magnitude when compared to the magnitude of the curvature shown in FIG. 3E.

FIG. 3G shows a cushion for a mask that includes two pillows. An exterior surface of the cushion is indicated. An edge of the surface is indicated. Dome and saddle regions are indicated.

FIG. 3H shows a cushion for a mask. An exterior surface of the cushion is indicated. An edge of the surface is indicated. A path on the surface between points A and B is indicated. A straight line distance between A and B is indicated. Two saddle regions and a dome region are indicated.

FIG. 3I shows the surface of a structure, with a one dimensional hole in the surface. The illustrated plane curve forms the boundary of a one dimensional hole.

FIG. 3J shows a cross-section through the structure of FIG. 3I. The illustrated surface bounds a two dimensional hole in the structure of FIG. 3I.

FIG. 3K shows a perspective view of the structure of FIG. 3I, including the two dimensional hole and the one dimensional hole. Also shown is the surface that bounds a two dimensional hole in the structure of FIG. 3I.

FIG. 3L shows a mask having an inflatable bladder as a cushion.

FIG. 3M shows a cross-section through the mask of FIG. 3L, and shows the interior surface of the bladder. The interior surface bounds the two dimensional hole in the mask.

FIG. 3N shows a further cross-section through the mask of FIG. 3L. The interior surface is also indicated.

FIG. 3O illustrates a left-hand rule.

FIG. 3P illustrates a right-hand rule.

FIG. 3Q shows a left ear, including the left ear helix.

FIG. 3R shows a right ear, including the right ear helix.

FIG. 3S shows a right-hand helix.

FIG. 3T shows a view of a mask, including the sign of the torsion of the space curve defined by the edge of the sealing membrane in different regions of the mask.

FIG. 3U shows a view of a plenum chamber 3200 showing a sagittal plane and a mid-contact plane.

FIG. 3V shows a view of a posterior of the plenum chamber of FIG. 3U. The direction of the view is normal to the mid-contact plane. The sagittal plane in FIG. 3V bisects the plenum chamber into left-hand and right-hand sides.

FIG. 3W shows a cross-section through the plenum chamber of FIG. 3V, the cross-section being taken at the sagittal plane shown in FIG. 3V. A ‘mid-contact’ plane is shown. The mid-contact plane is perpendicular to the sagittal plane. The orientation of the mid-contact plane corresponds to the orientation of a chord 3210 which lies on the sagittal plane and just touches the cushion of the plenum chamber at two points on the sagittal plane: a superior point 3220 and an inferior point 3230. Depending on the geometry of the cushion in this region, the mid-contact plane may be a tangent at both the superior and inferior points.

FIG. 3X shows the plenum chamber 3200 of FIG. 3U in position for use on a face. The sagittal plane of the plenum chamber 3200 generally coincides with the midsagittal plane of the face when the plenum chamber is in position for use. The mid-contact plane corresponds generally to the ‘plane of the face’ when the plenum chamber is in position for use. In FIG. 3X the plenum chamber 3200 is that of a nasal mask, and the superior point 3220 sits approximately on the sellion, while the inferior point 3230 sits on the lip superior.

### 4.4 RPT Device

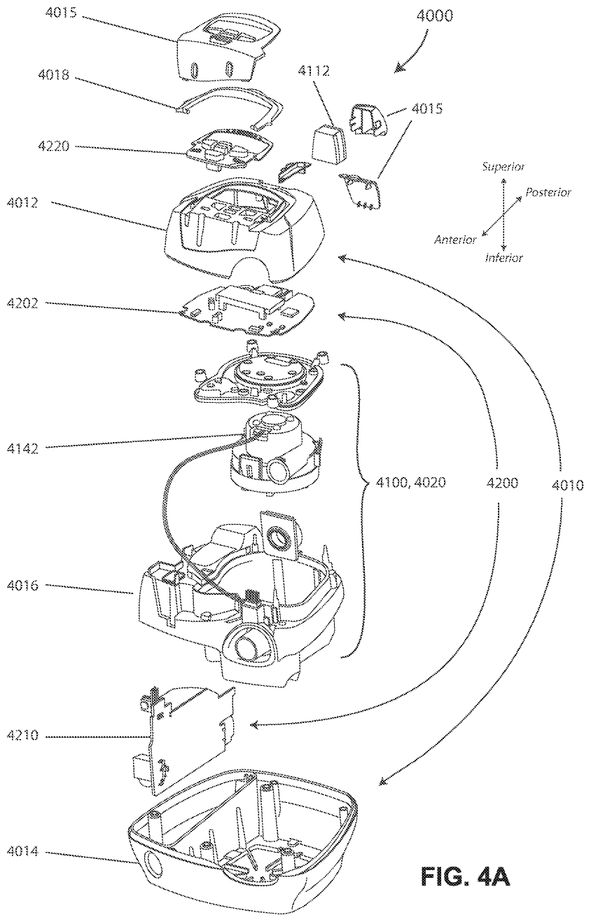

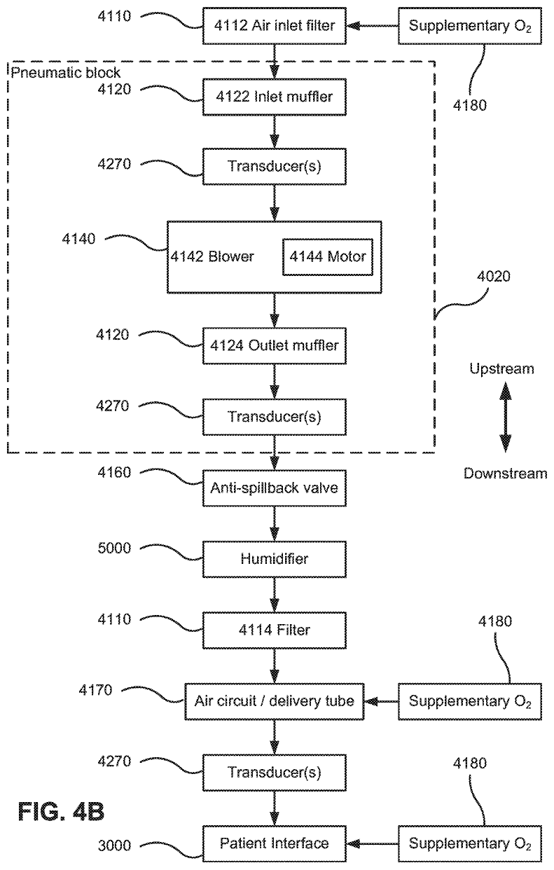

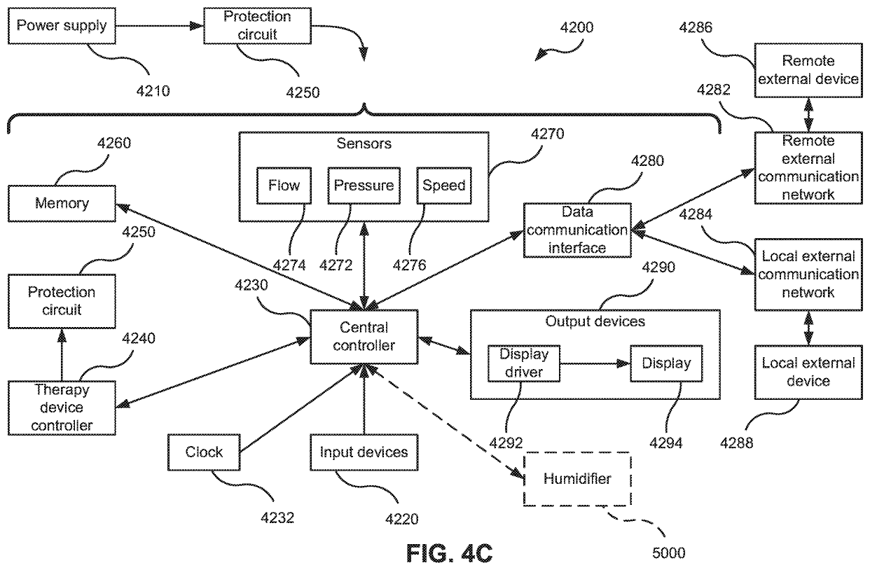

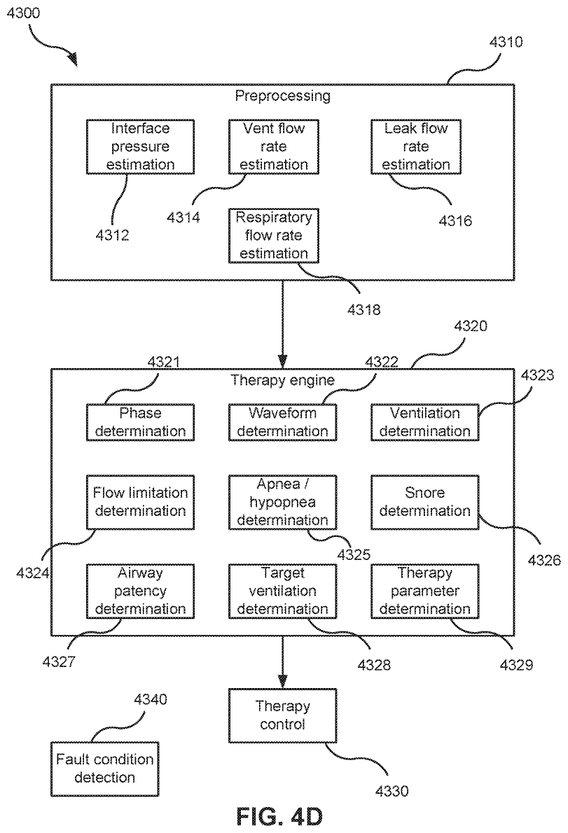

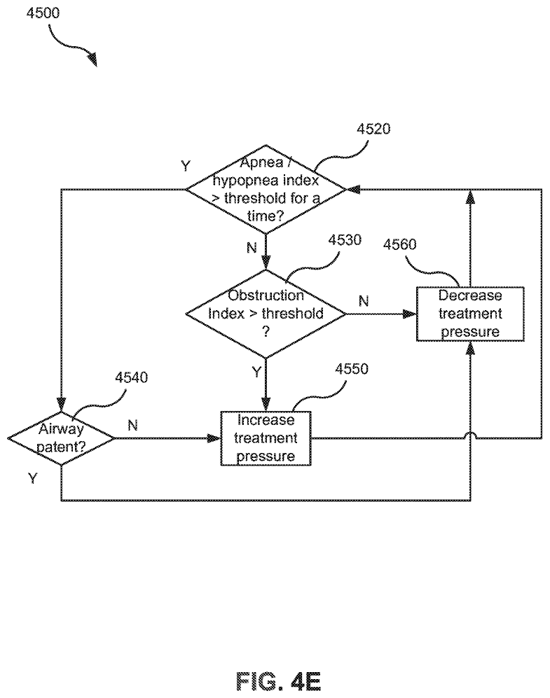

FIG. 4A shows an RPT device in accordance with one form of the present technology.

FIG. 4B is a schematic diagram of the pneumatic path of an RPT device in accordance with one form of the present technology. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

FIG. 4C is a schematic diagram of the electrical components of an RPT device in accordance with one form of the present technology.

FIG. 4D is a schematic diagram of the algorithms implemented in an RPT device in accordance with one form of the present technology.

FIG. 4E is a flow chart illustrating a method carried out by the therapy engine module of FIG. 4D in accordance with one form of the present technology.

### 4.5 Humidifier

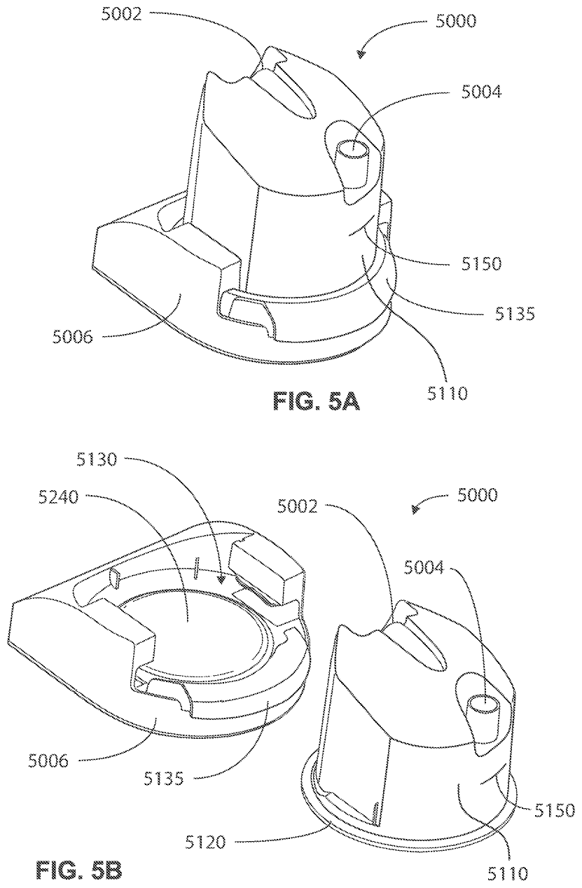

FIG. 5A shows an isometric view of a humidifier in accordance with one form of the present technology.

FIG. 5B shows an isometric view of a humidifier in accordance with one form of the present technology, showing a humidifier reservoir 5110 removed from the humidifier reservoir dock 5130.

### 4.6 Breathing Waveforms

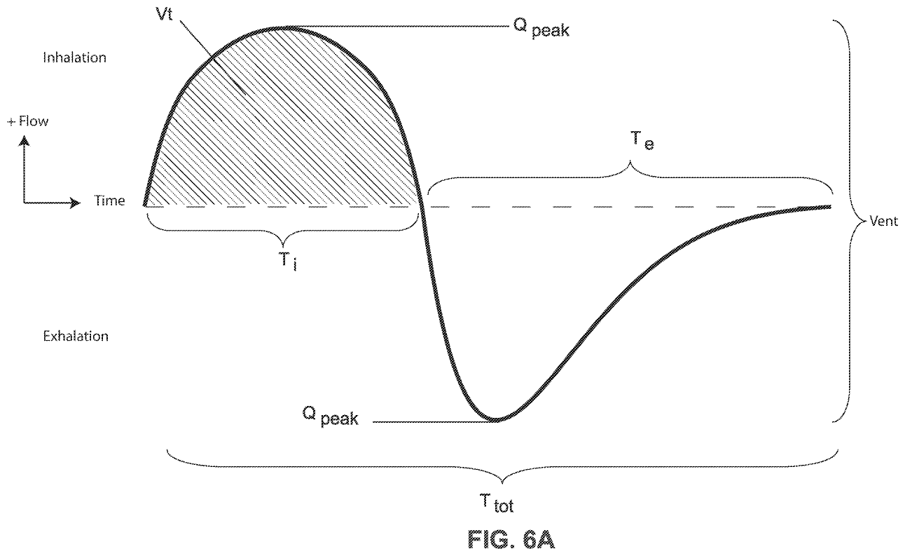

FIG. 6A shows a model typical breath waveform of a person while sleeping.

### 4.7 Interface with Motor

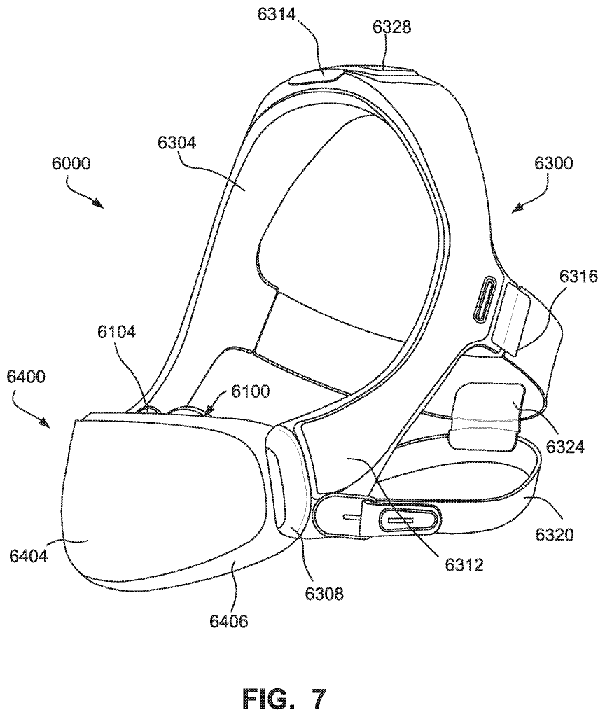

FIG. 7 illustrates a perspective view of a first version of a full-face patient interface.

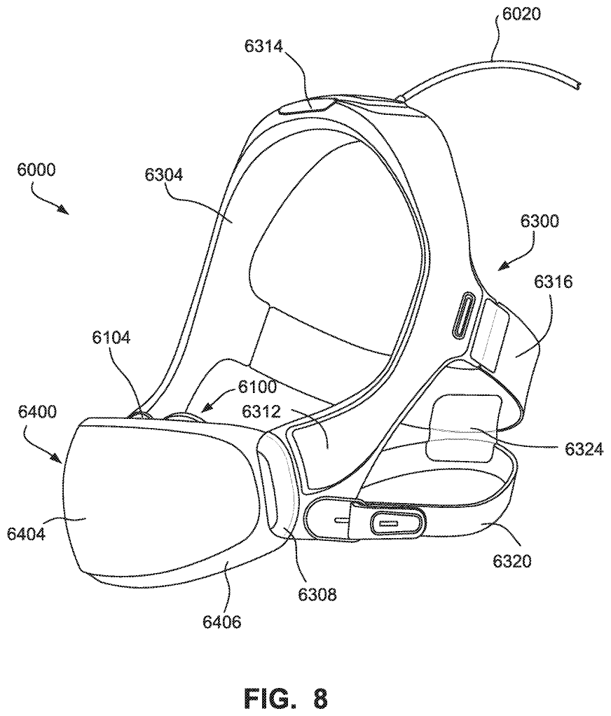

FIG. 8 illustrates a perspective view of the full-face patient interface of FIG. 7 connected to a power cord.

FIG. 9 illustrates a perspective view of the full-face patient interface of FIG. 7 with an alternate positioning and stabilising structure.

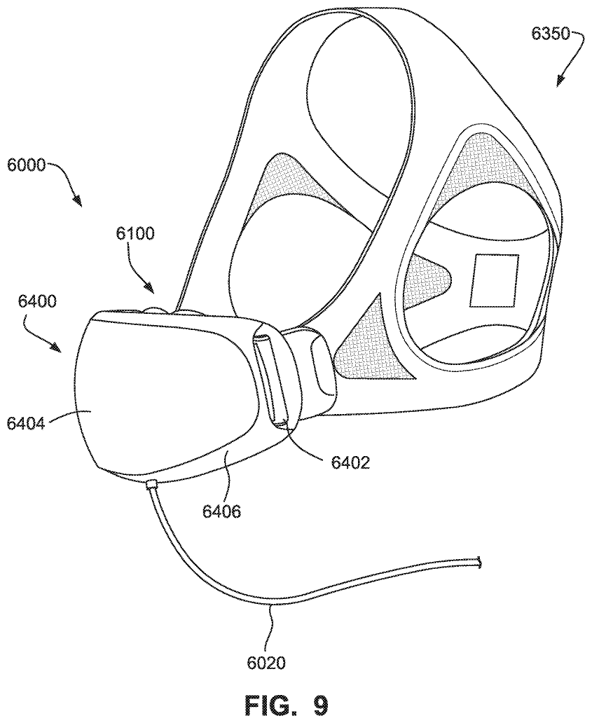

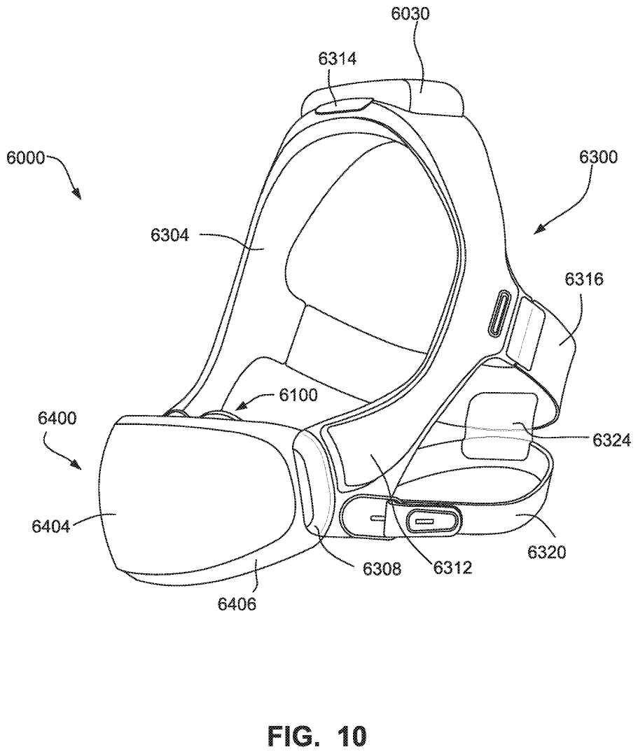

FIG. 10 illustrates a perspective view of the full-face patient interface of FIG. 7 connected to a battery.

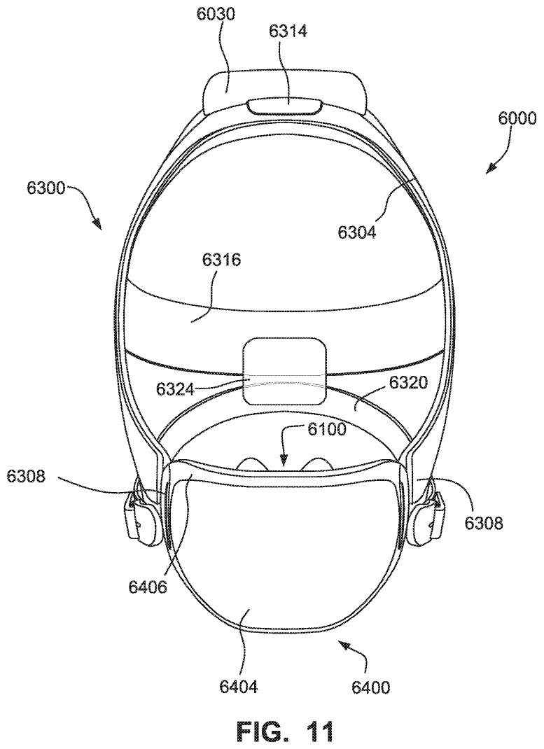

FIG. 11 illustrates a front view of the full-face patient interface and battery of FIG. 10.

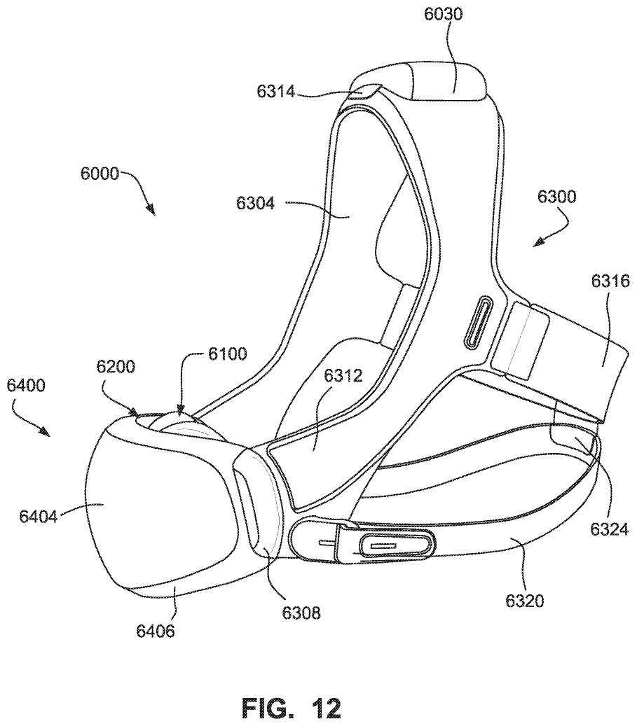

FIG. 12 illustrates a side view of the full-face patient interface and battery of FIG. 10.

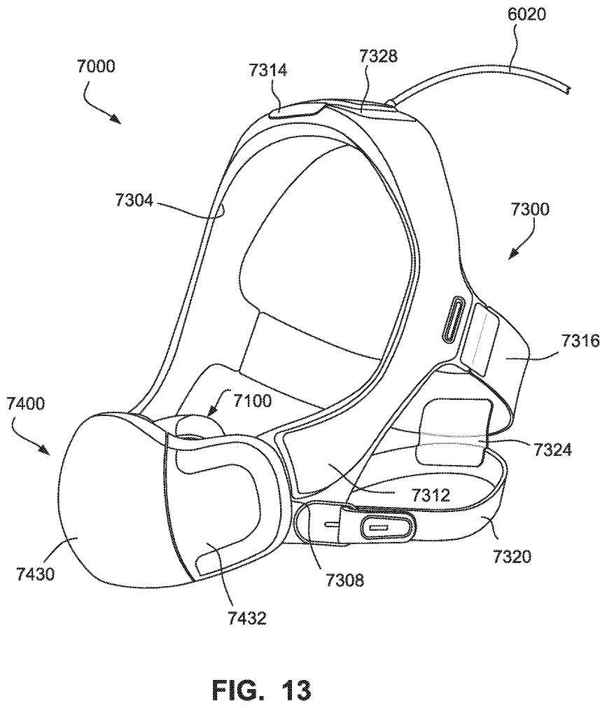

FIG. 13 illustrates a perspective view of a second version of a full-face patient interface connected to a power cord.

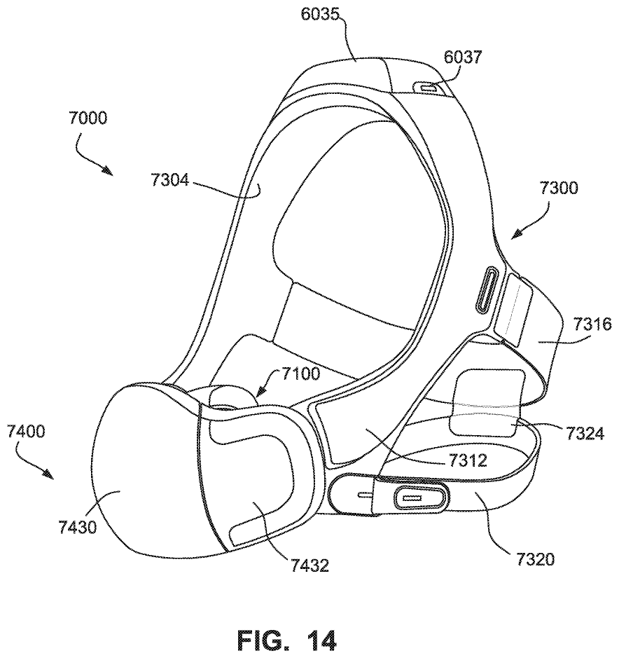

FIG. 14 illustrates a perspective view of the full-face patient interface of FIG. 13 connected to a battery.

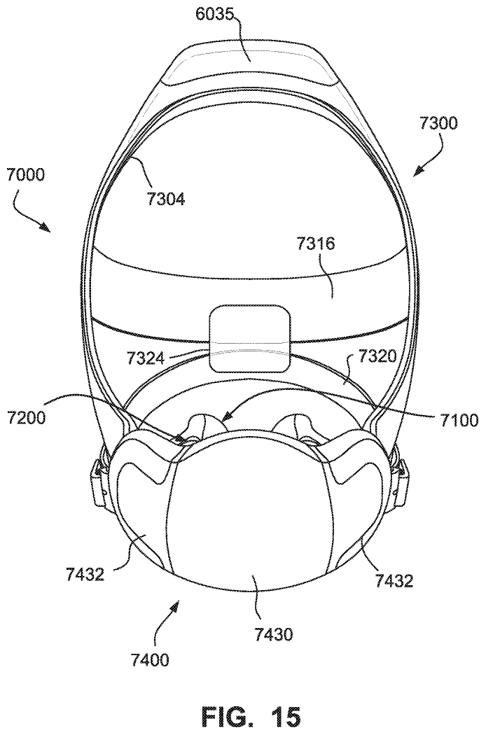

FIG. 15 illustrates a front view of the full-face patient interface and battery of FIG. 14.

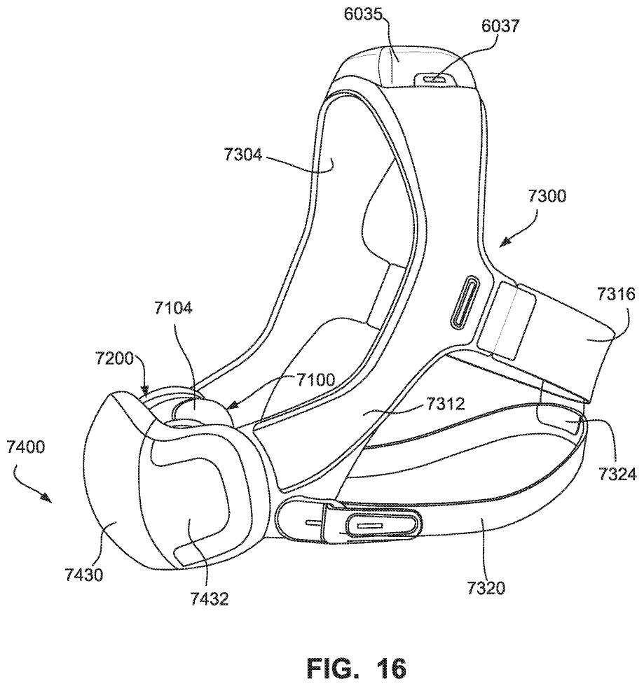

FIG. 16 illustrates a side view of the full-face patient interface and battery of FIG. 14.

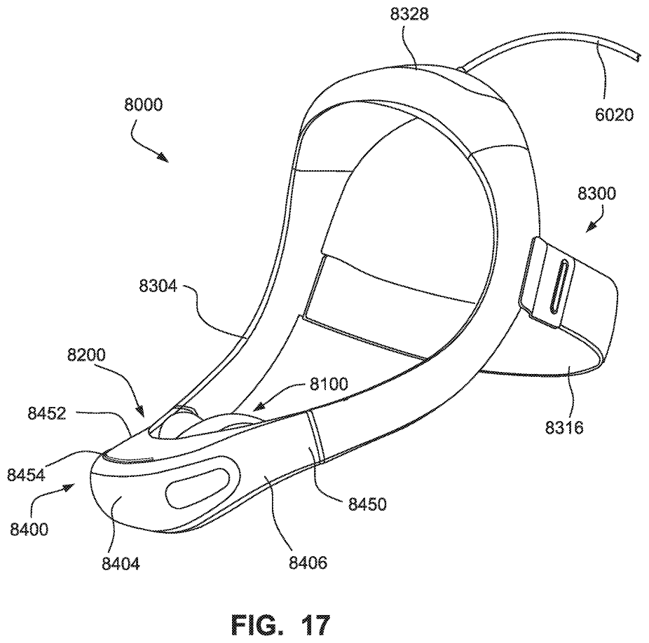

FIG. 17 illustrates a perspective view of a first version of a nasal patient interface connected to a power cord.

FIG. 18 illustrates a perspective view of the nasal patient interface of FIG. 17 connected to a battery.

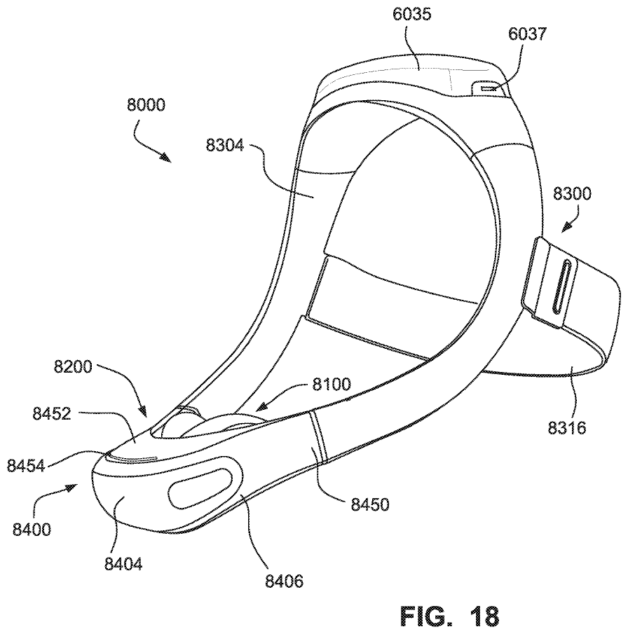

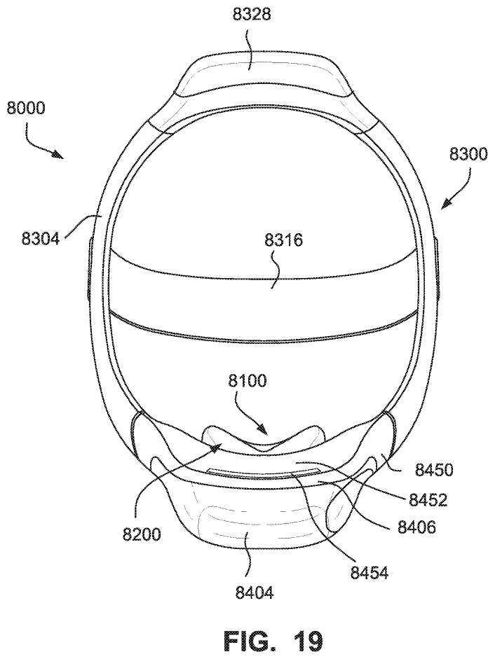

FIG. 19 illustrates a front view of the nasal patient interface and battery of FIG. 18.

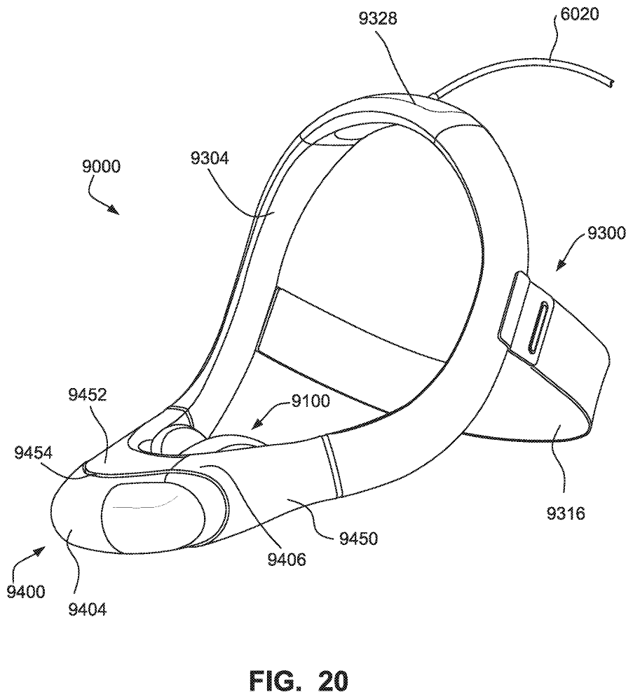

FIG. 20 illustrates a perspective view of a second version of a nasal patient interface connected to a power cord.

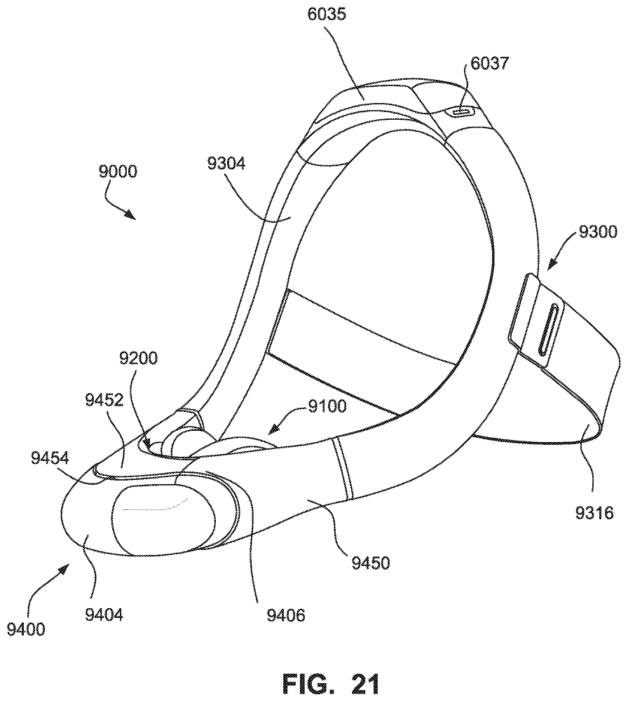

FIG. 21 illustrates a perspective view of the nasal patient interface of FIG. 20 connected to a battery.

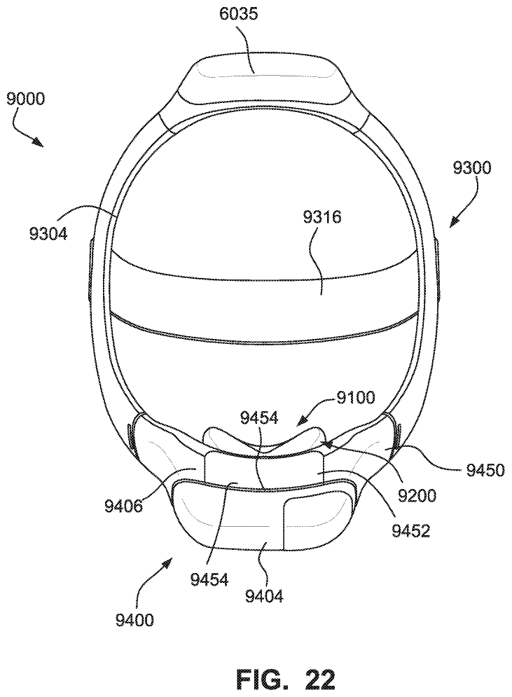

FIG. 22 illustrates a front view of the nasal patient interface and battery of FIG. 21.

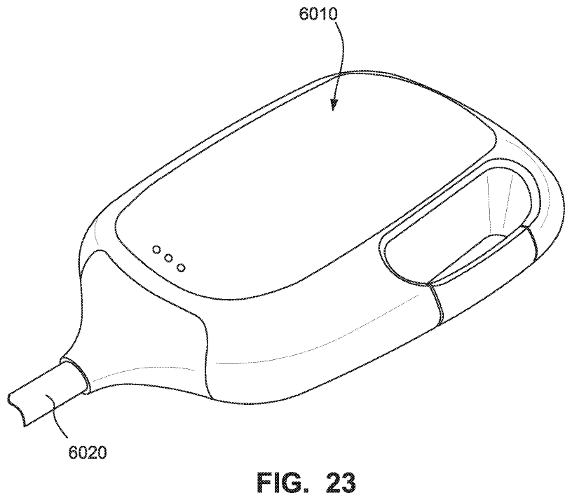

FIG. 23 illustrates a perspective view of a battery connected to the power cord.

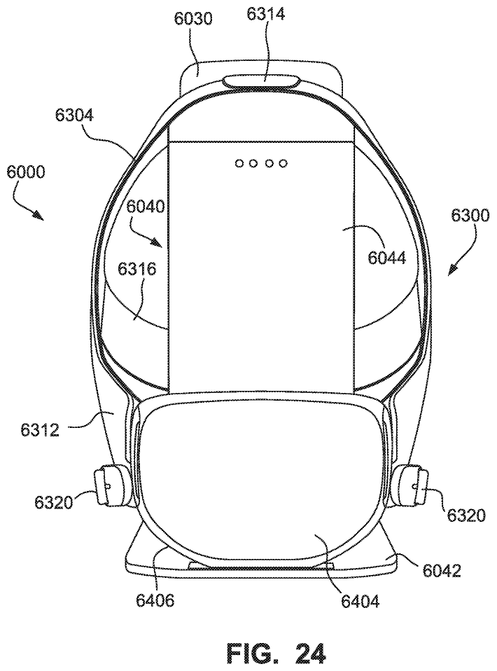

FIG. 24 illustrates a front view of full-face patient interface and battery of FIG. 10 connected to a charger.

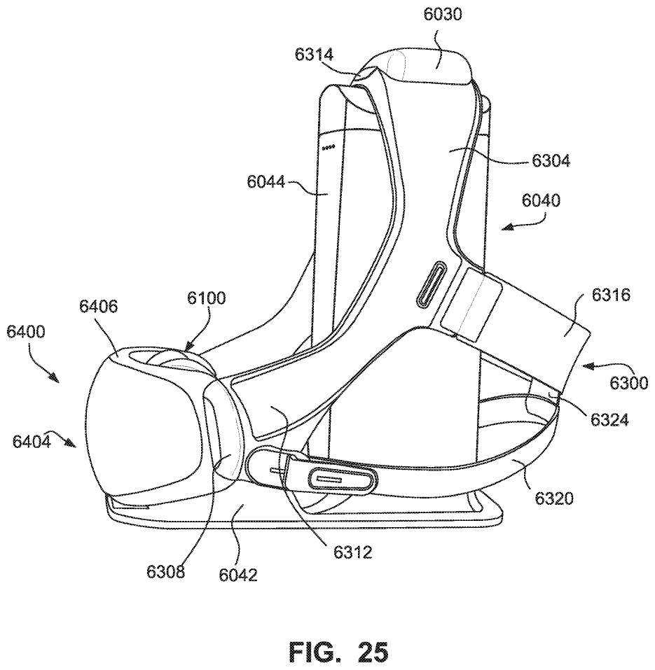

FIG. 25 illustrates a side view of the full-face patient interface and charger of FIG. 24.

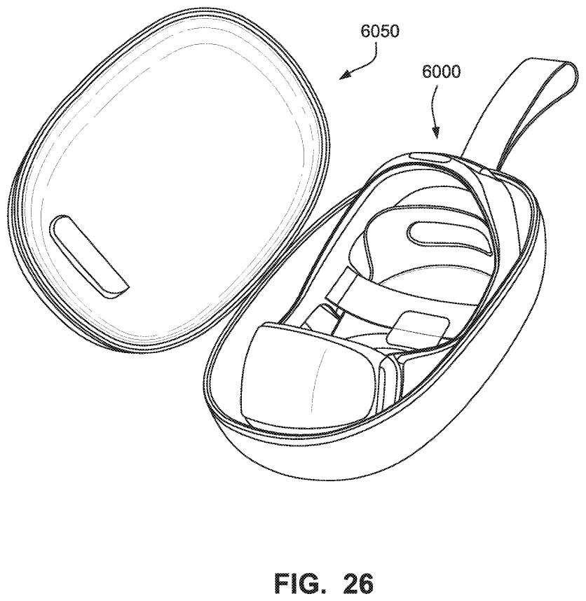

FIG. 26 illustrates a perspective view of a case for storing the patient interface of FIG. 7.

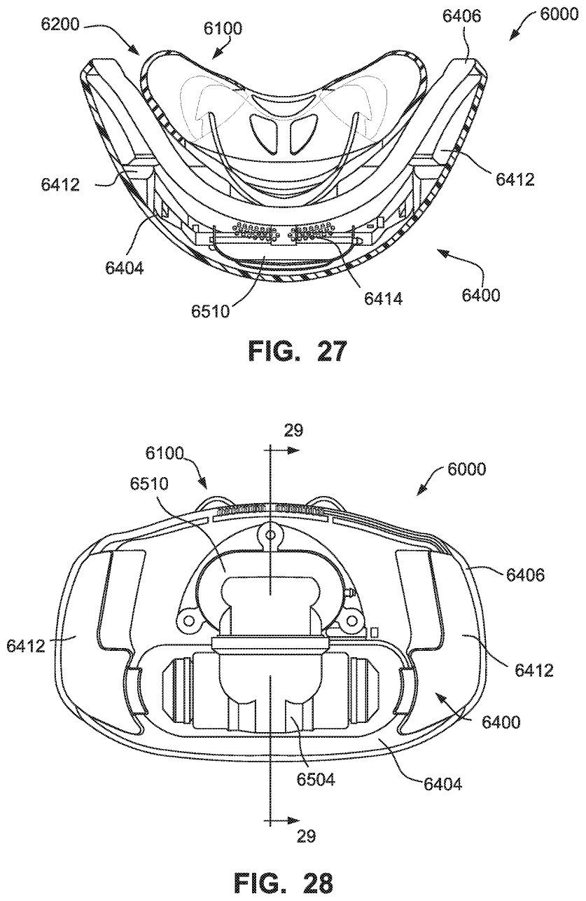

FIG. 27 is a top view of a full-face patient interface with a transparent outer casing in order to view the internal elements.

FIG. 28 is a front view of the full-face patient interface of FIG. 27.

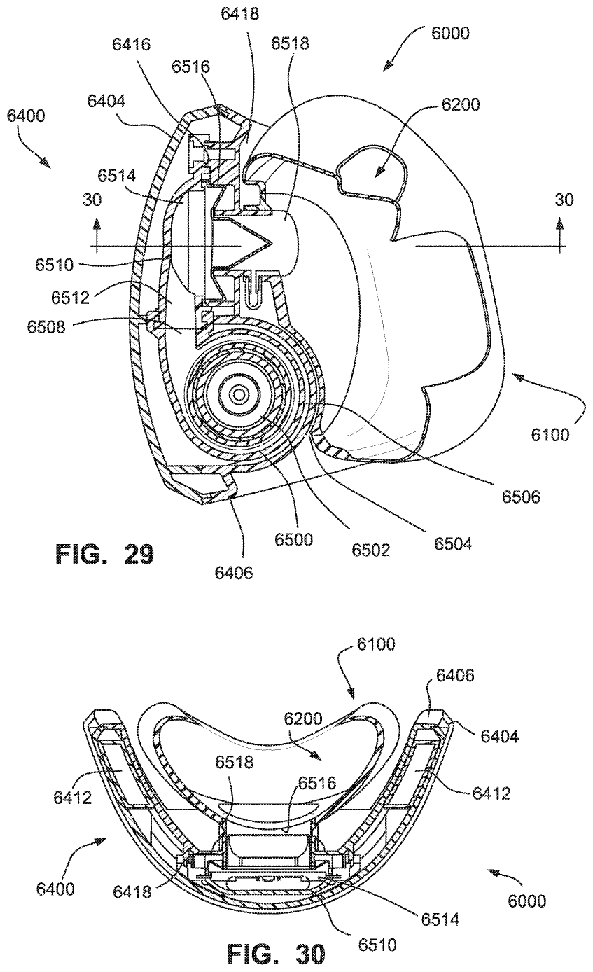

FIG. 29 is a cross-sectional view of the full-face patient interface of FIG. 28 illustrating components of an RPT device contained within the patient interface.

FIG. 30 is a cross-sectional view of the full-face patient interface of FIG. 29 illustrating components of the RPT device contained within the patient interface.

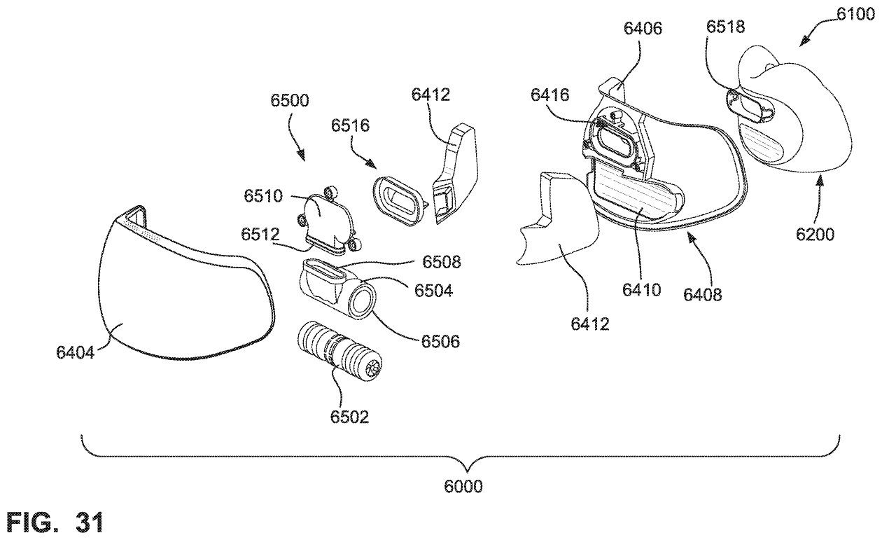

FIG. 31 is an exploded view of the patient interface of FIG. 27, illustrating the components of the RPT device.

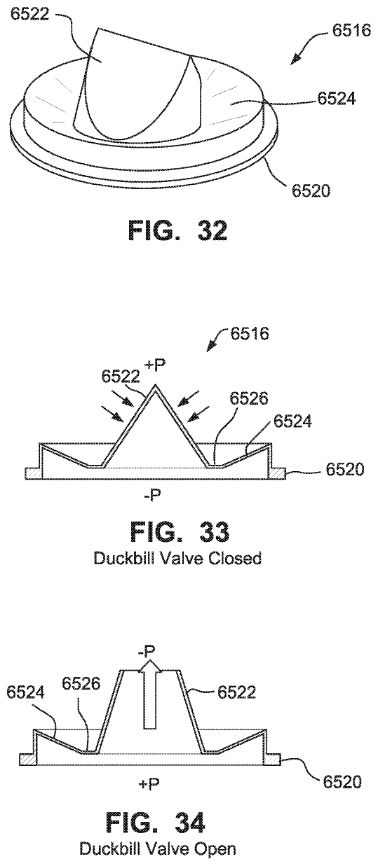

FIG. 32 is a perspective view of a valve.

FIG. 33 is a cross-sectional view of the valve of FIG. 32 illustrating the valve in a closed position.

FIG. 34 is a cross-sectional view of the valve of FIG. 32 illustrating the valve in an open position.

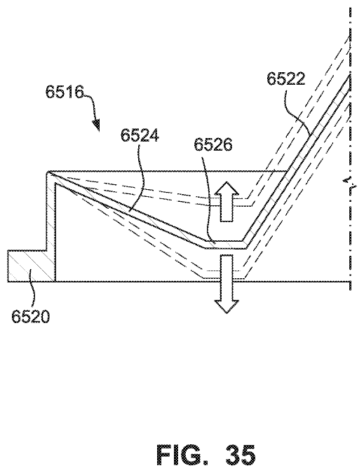

FIG. 35 is a detail view of the valve in FIGS. 33 and 34.

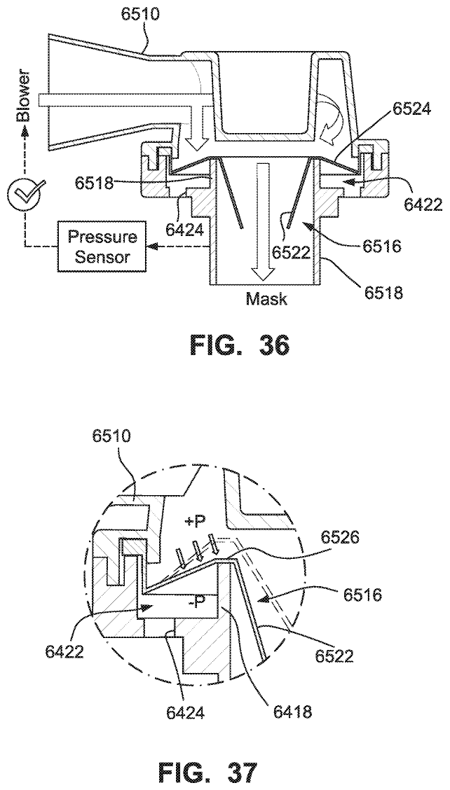

FIG. 36 is a cross-sectional view of the valve of FIG. 32 connected to the patient interface and moved to the open position during inspiration of a patient.

FIG. 37 is a detail view of the cross-sectional view of FIG. 36.

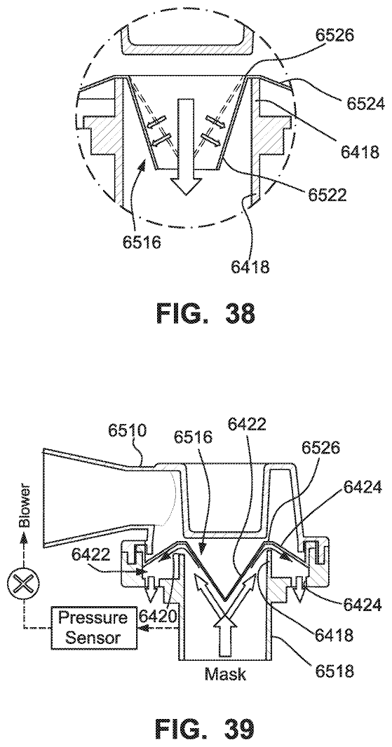

FIG. 38 is a detail view of the cross-sectional view of FIG. 36.

FIG. 39 is a cross-sectional view of the valve of FIG. 32 connected to the patient interface and moved to the closed position during expiration of a patient.

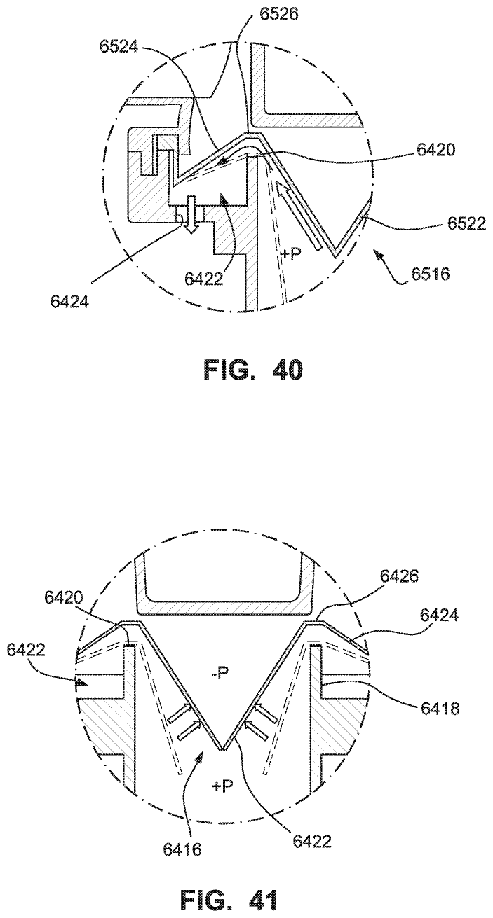

FIG. 40 is a detail view of the cross-sectional view of FIG. 39.

FIG. 41 is a detail view of the cross-sectional view of FIG. 39.

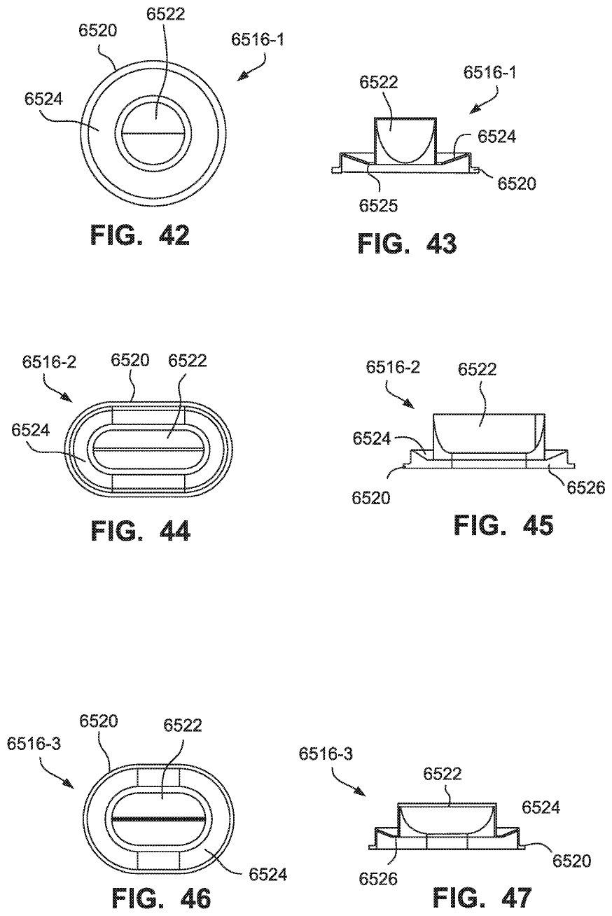

FIG. 42 is a top view of a first example of a valve.

FIG. 43 is a cross-sectional view of the valve of FIG. 42.

FIG. 44 is a top view of a second example of a valve.

FIG. 45 is a cross-sectional view of the valve of FIG. 44.

FIG. 46 is a top view of a third example of a valve.

FIG. 47 is a cross-sectional view of the valve of FIG. 46.

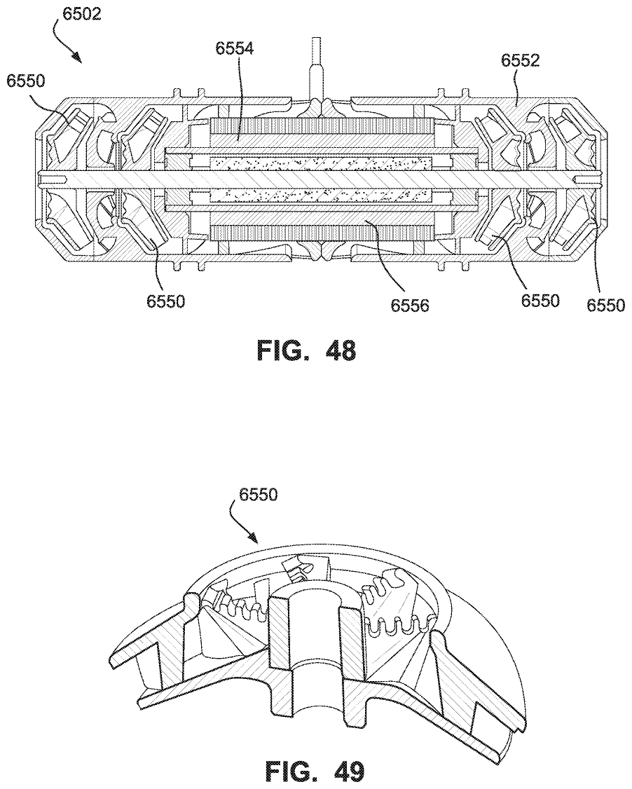

FIG. 48 is a cross-sectional view of a blower.

FIG. 49 is a partial cross-sectional view of an impeller used with the blower of FIG. 48.

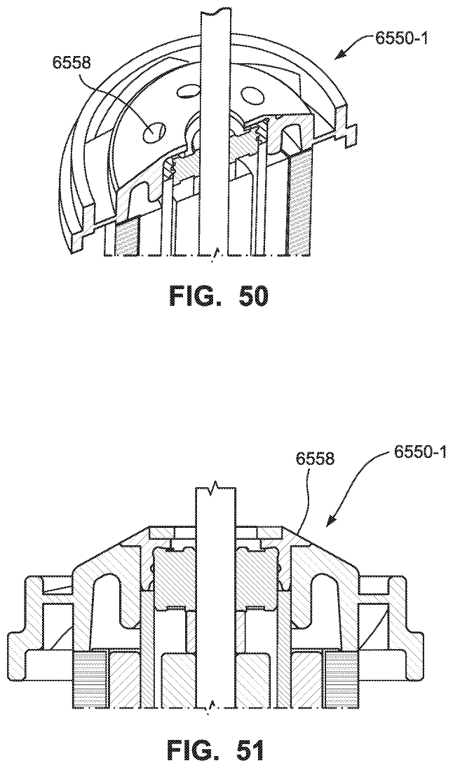

FIG. 50 is a perspective cross-sectional view of an alternate form of an impeller usable with the blower of FIG. 48.

FIG. 51 is a cross-sectional view of an alternate form of an impeller usable with the blower of FIG. 48.

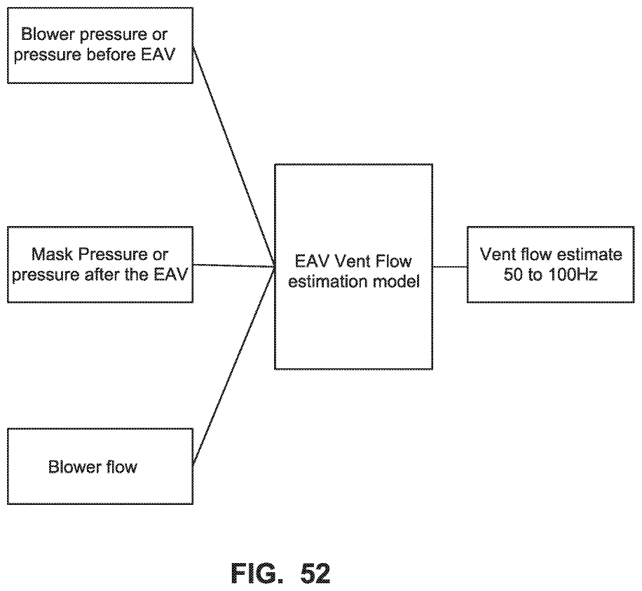

FIG. 52 is a schematic view of the operation of the valve of FIG. 32 and the blower of FIG. 48.

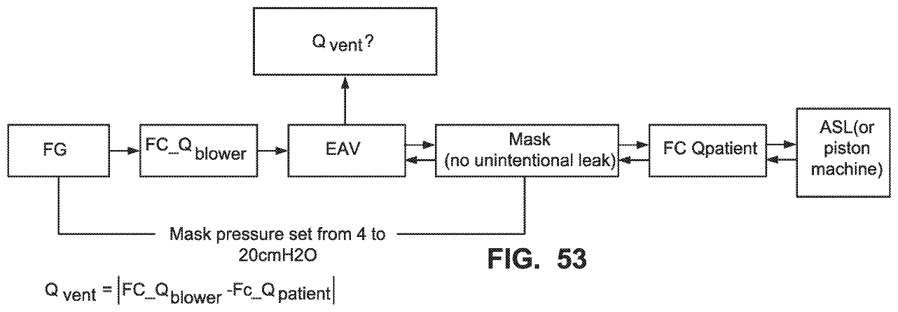

FIG. 53 is a flow diagram of the flow through the valve of FIG. 32.

### 4.8 AR/VR Interface

FIG. 54 is a front perspective view of a head-mounted display.

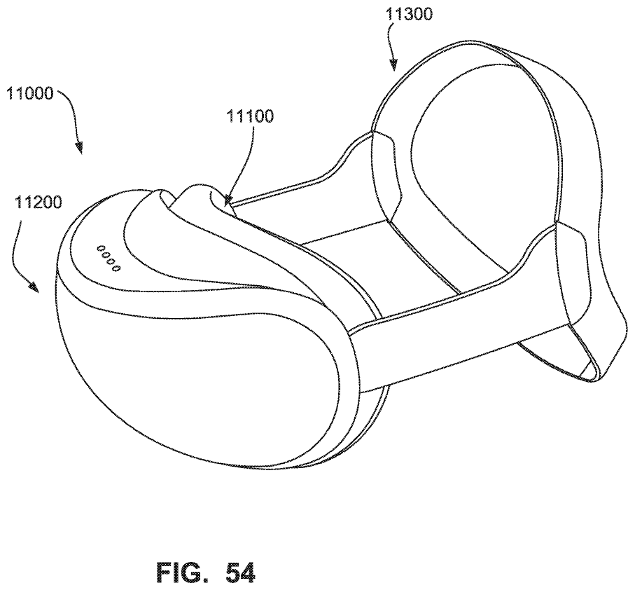

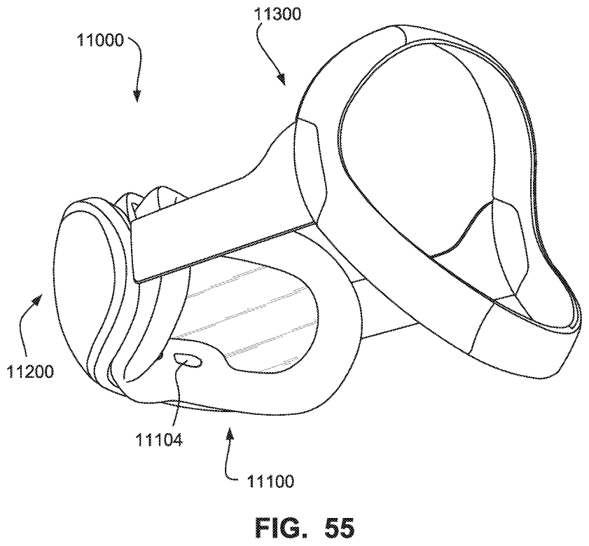

FIG. 55 is a rear perspective view of the head-mounted display of FIG. 54.

### 4.9 Interface with Speaker

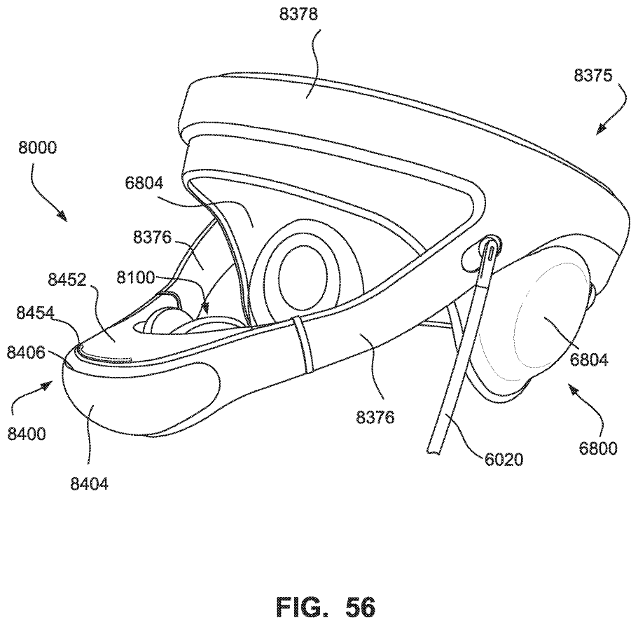

FIG. 56 is a perspective view of a patient interface with an audio system.

### 4.10 Alternative Examples of Interfaces

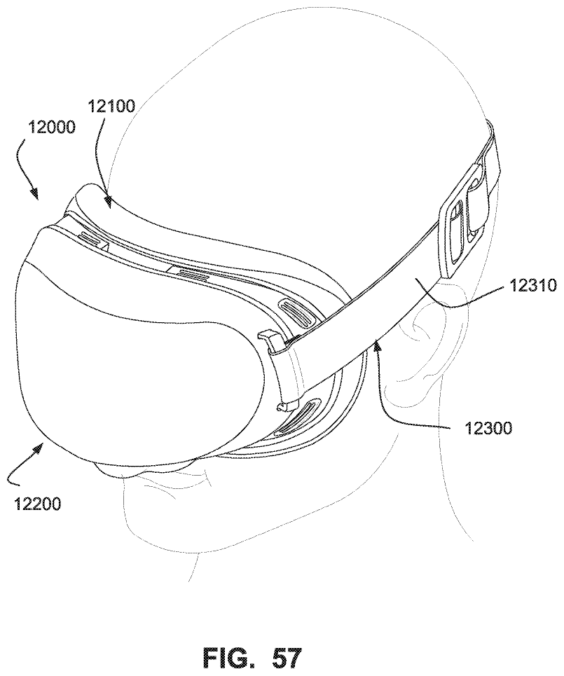

FIG. 57 is a perspective view of a patient interface including an RPT device, a head-mounted display interface and a user interface structure on a patient's head according to an example of the present technology.

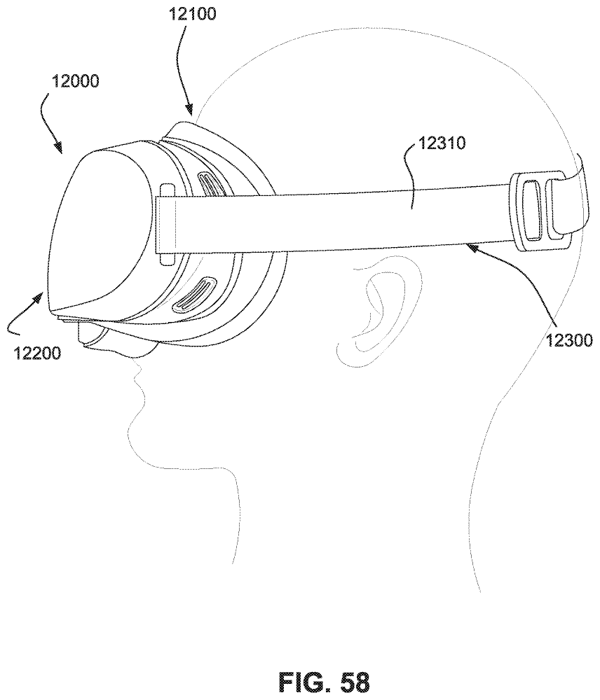

FIG. 58 is a side view of the patient interface of FIG. 57 on a patient's head according to an example of the present technology.

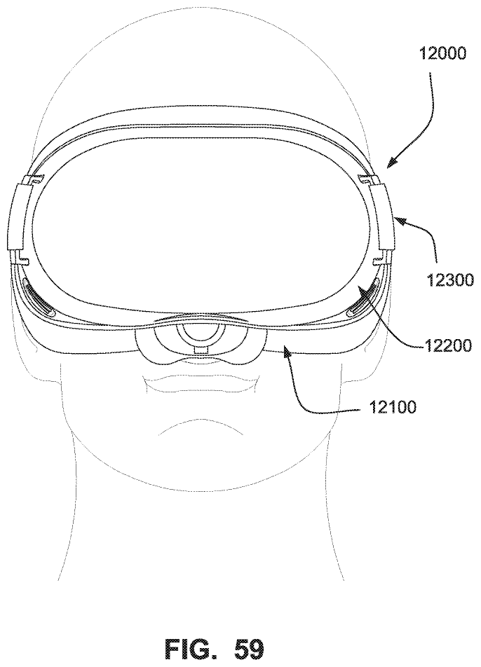

FIG. 59 is a front view of the patient interface of FIG. 57 on a patient's head according to an example of the present technology.

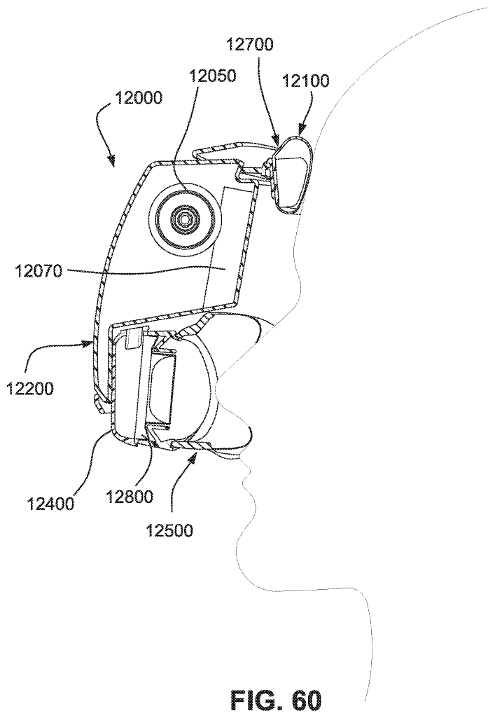

FIG. 60 is a cross-sectional view of the patient interface of FIG. 57 on a patient's head according to an example of the present technology.

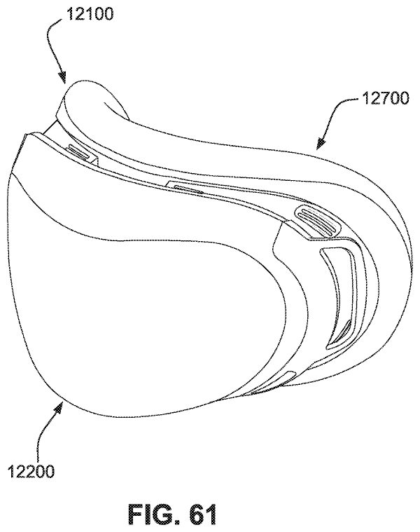

FIG. 61 is a front perspective view of the patient interface of FIG. 57 according to an example of the present technology.

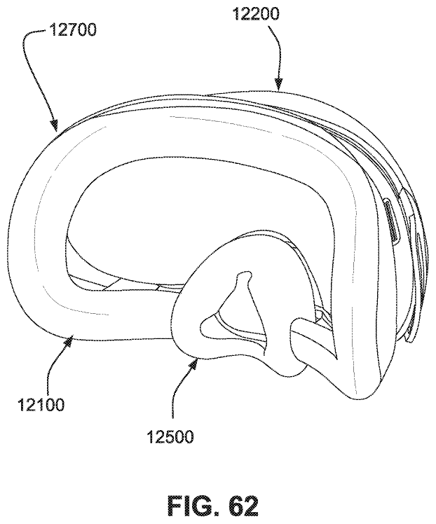

FIG. 62 is a rear perspective view of the patient interface of FIG. 57 according to an example of the present technology.

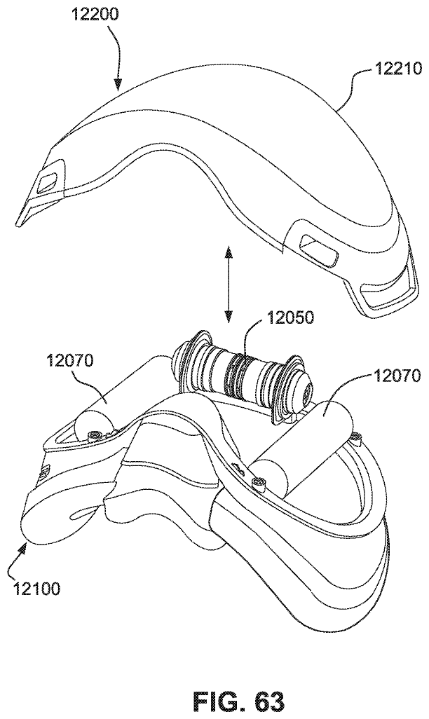

FIG. 63 is a front perspective view of the patient interface of FIG. 57 with the front case removed according to an example of the present technology.

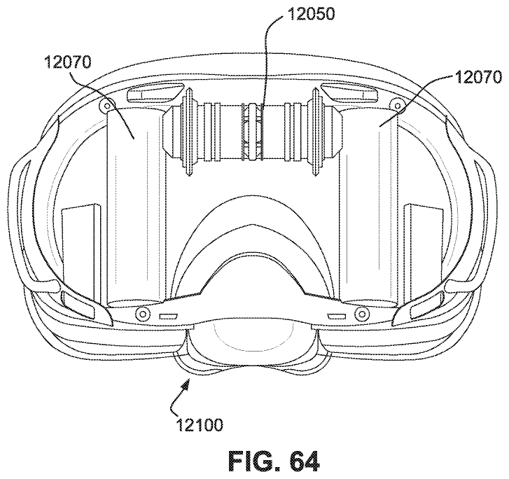

FIG. 64 is a front view of the patient interface of FIG. 57 without the front case according to an example of the present technology.

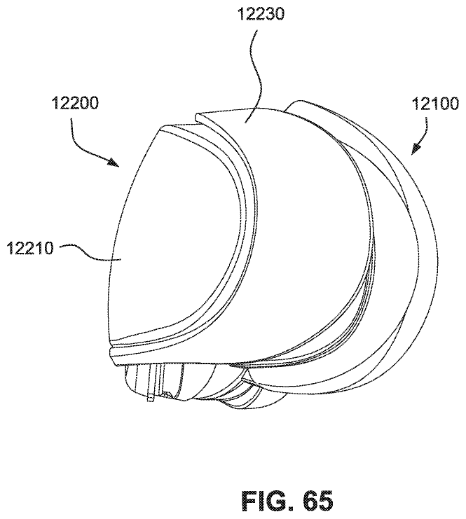

FIG. 65 is a side view of a patient interface including an RPT device, a head-mounted display interface and a user interface structure according to an example of the present technology.

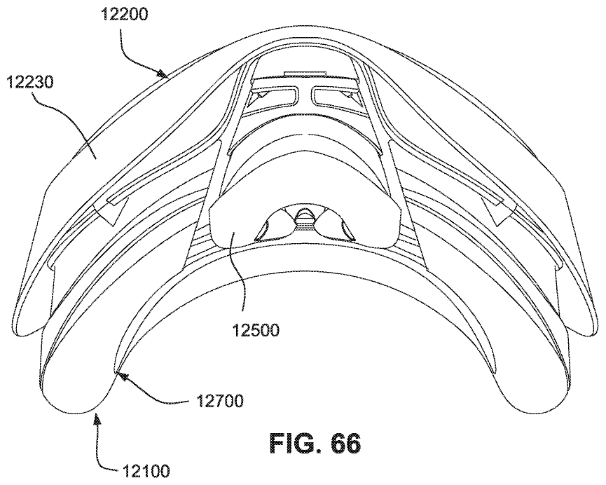

FIG. 66 is a bottom view of the patient interface of FIG. 65 according to an example of the present technology.

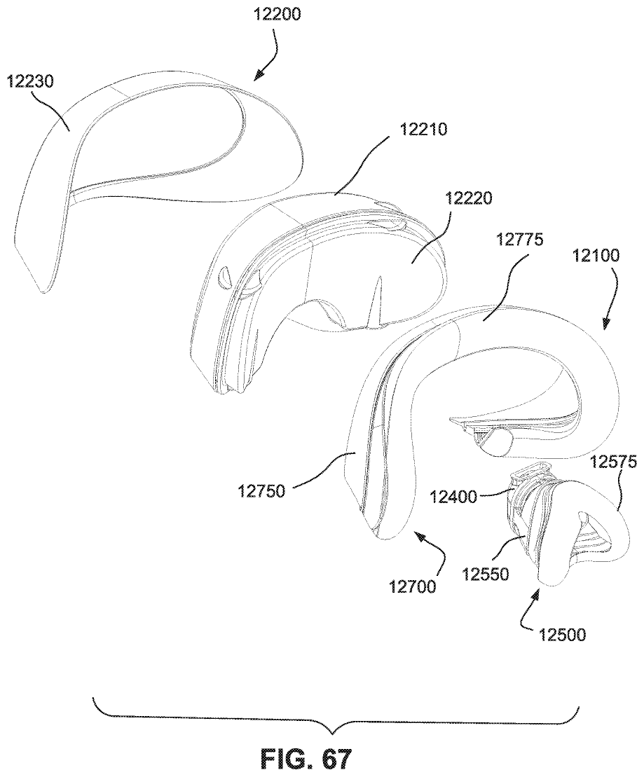

FIG. 67 is a rear exploded view of the patient interface of FIG. 65 according to an example of the present technology.

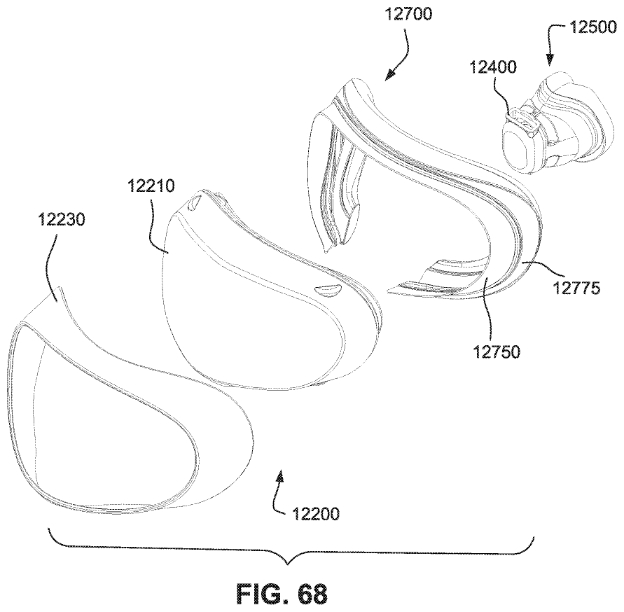

FIG. 68 is a front exploded view of the patient interface of FIG. 65 according to an example of the present technology.

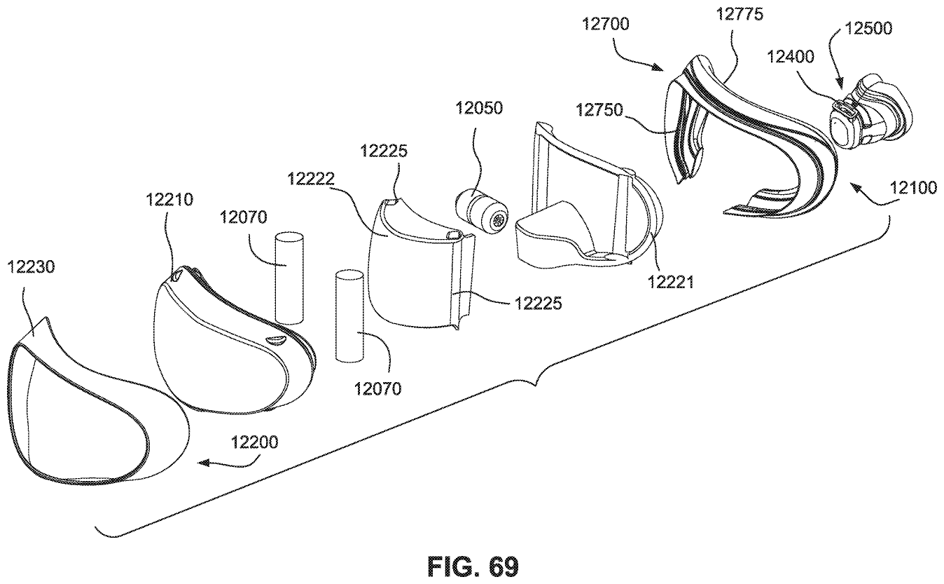

FIG. 69 is another front exploded view of the patient interface of FIG. 65 according to an example of the present technology.

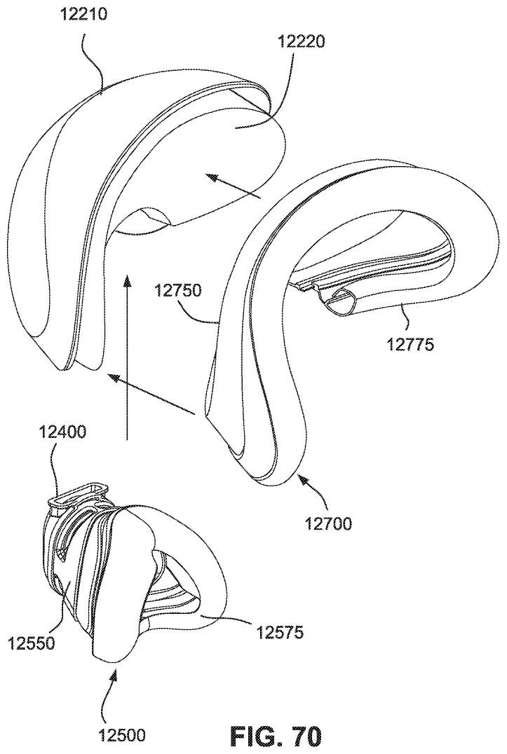

FIG. 70 is an exploded view of the user interface structure and the casing of the patient interface of FIG. 65 according to an example of the present technology.

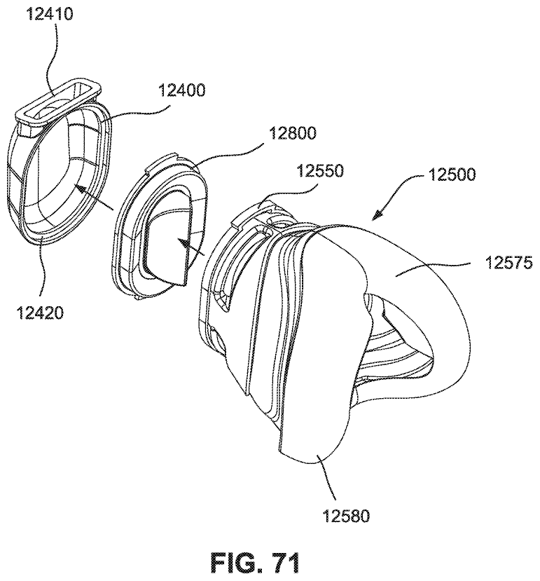

FIG. 71 is an exploded view of a therapy cushion component, EAV, and manifold according to an example of the present technology.

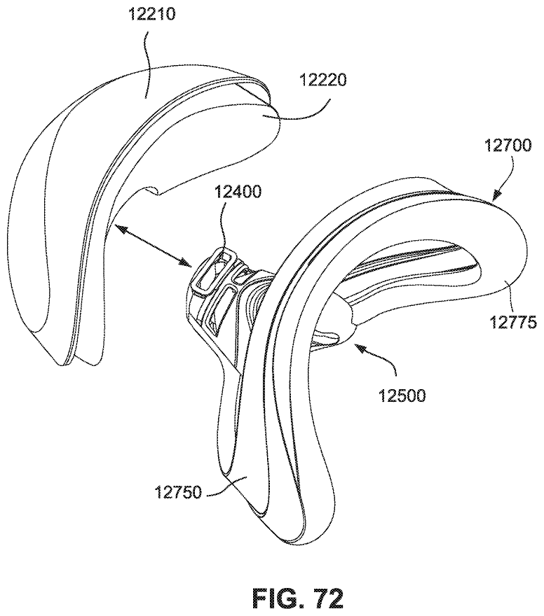

FIG. 72 is an exploded view of a user interface structure and a casing of a patient interface according to an example of the present technology.

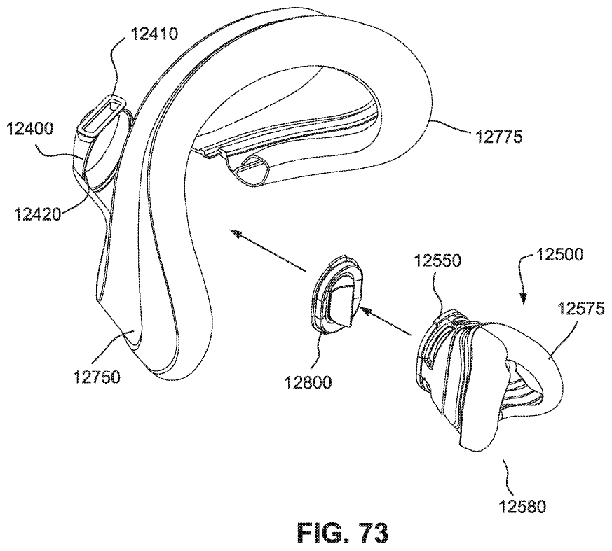

FIG. 73 is an exploded view of the user interface structure and EAV of FIG. 72.

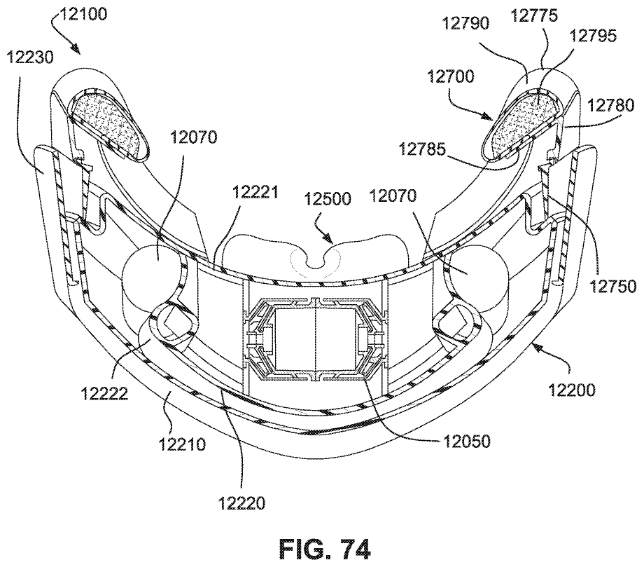

FIG. 74 is a cross-sectional view of a patient interface according to an example of the present technology.

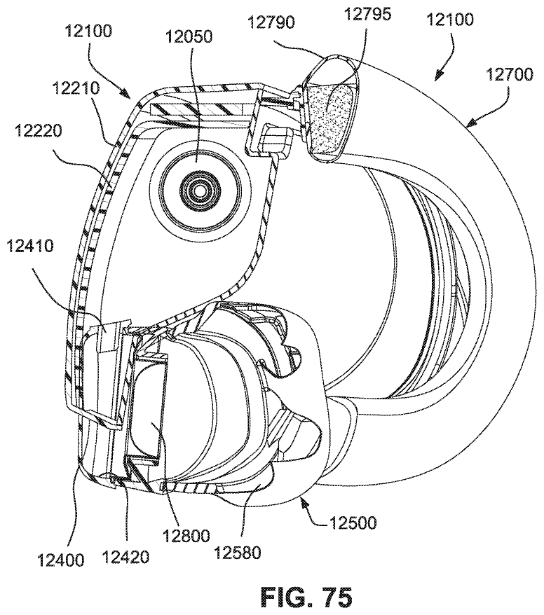

FIG. 75 is a cross-sectional view of a patient interface according to an example of the present technology.

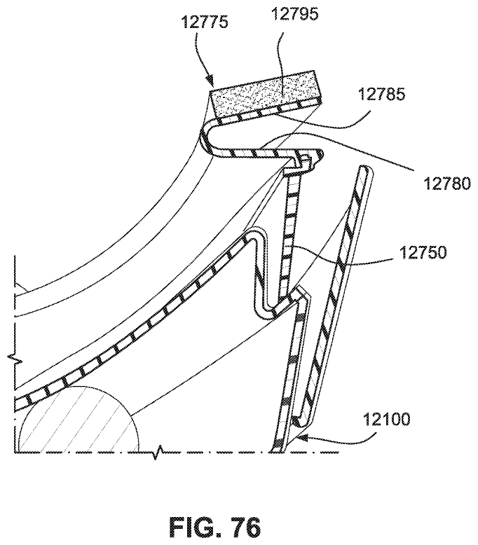

FIG. 76 is a cross-sectional view of a patient interface according to an example of the present technology.

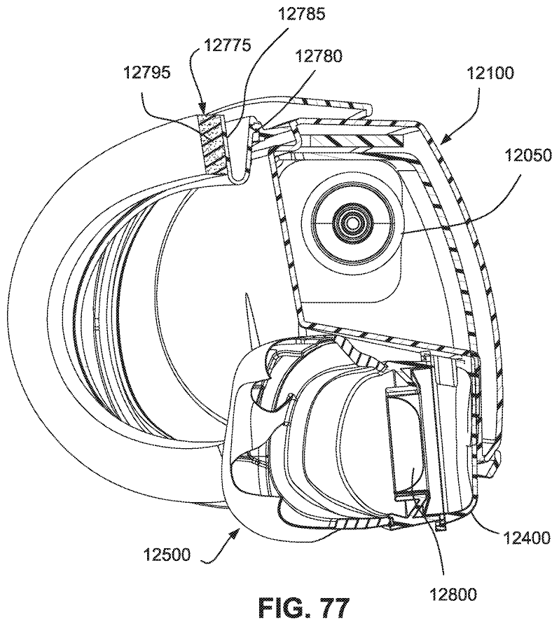

FIG. 77 is a cross-sectional view of a patient interface according to an example of the present technology.

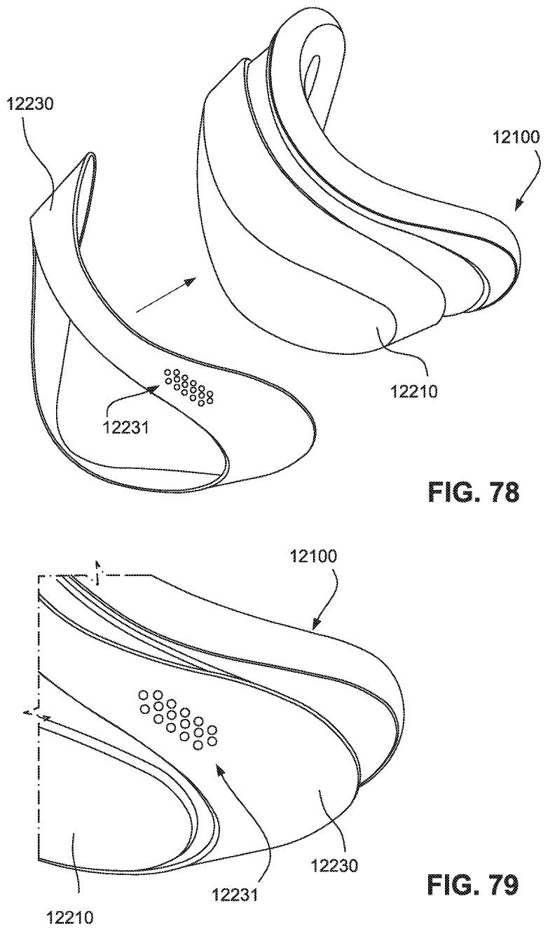

FIG. 78 is an exploded view of a shroud for a casing according to an example of the present technology.

FIG. 79 is an enlarged perspective view of the shroud of FIG. 78 according to an example of the present technology.

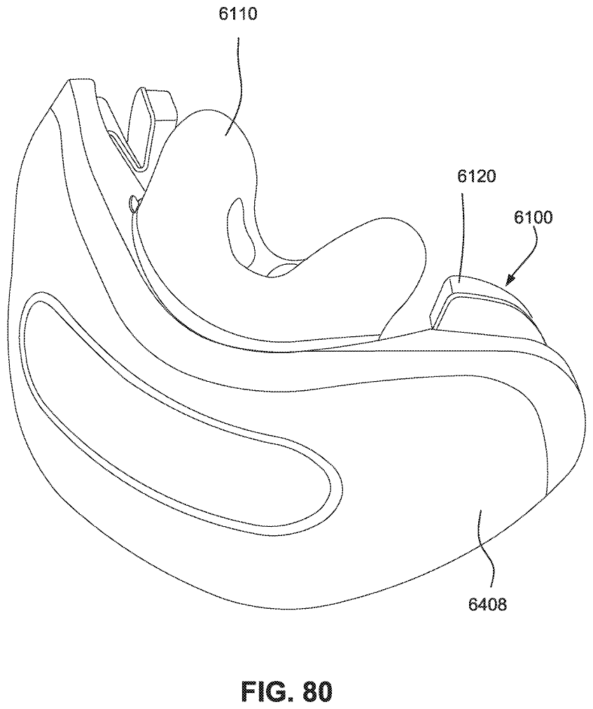

FIG. 80 is a front perspective view of a full-face patient interface according to an example of the present technology.

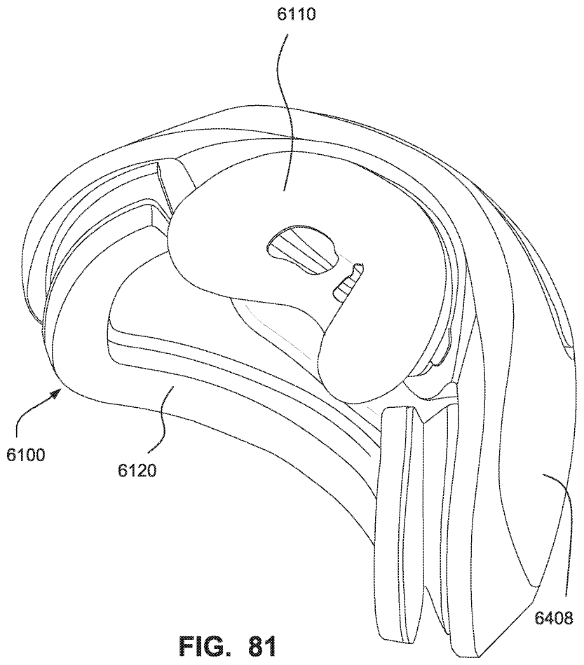

FIG. 81 is a rear perspective view of the full-face patient interface of FIG. 80 according to an example of the present technology.

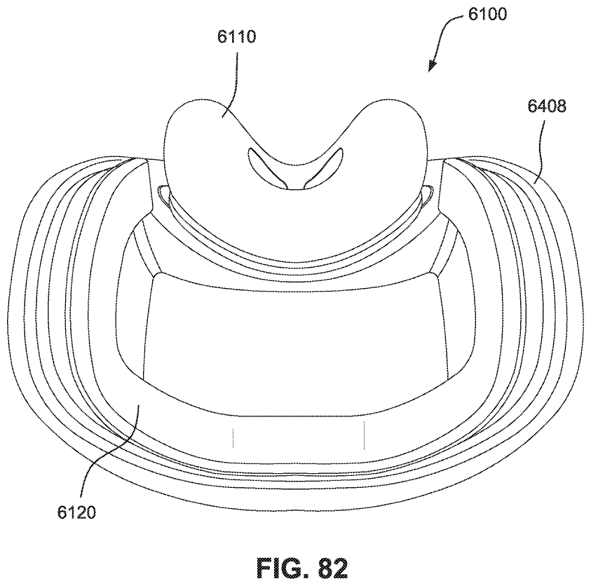

FIG. 82 is a rear view of the full-face patient interface of FIG. 80 according to an example of the present technology.

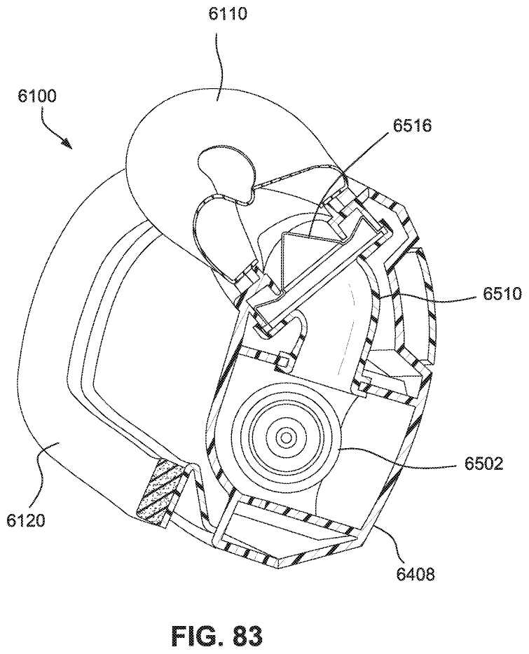

FIG. 83 is a cross-sectional view of the full-face patient interface of FIG. 80 according to an example of the present technology.

### 5 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 5.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 5.2 Respiratory Therapy Systems

In one form, the present technology comprises a respiratory therapy system for treating a respiratory disorder. The respiratory therapy system may comprise an RPT device 4000 for supplying a flow of air to the patient 1000 via an air circuit 4170 and a patient interface 3000.

### 5.3 Patient Interface

A non-invasive patient interface 3000 in accordance with one aspect of the present technology comprises the following functional aspects: a seal-forming structure 3100, a plenum chamber 3200, a positioning and stabilising structure 3300, a vent 3400, one form of connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

If a patient interface is unable to comfortably deliver a minimum level of positive pressure to the airways, the patient interface may be unsuitable for respiratory pressure therapy.

The patient interface 3000 in accordance with one form of the present technology is constructed and arranged to be able to provide a supply of air at a positive pressure above the ambient.

The patient interface 3000 in accordance with one form of the present technology is constructed and arranged to be able to provide a supply of air at a positive pressure of at least 2 cmH2O with respect to ambient.

The patient interface 3000 in accordance with one form of the present technology is constructed and arranged to be able to provide a supply of air at a positive pressure of at least 4 cmH2O with respect to ambient.

The patient interface 3000 in accordance with one form of the present technology is constructed and arranged to be able to provide a supply of air at a positive pressure of at least 6 cmH2O with respect to ambient.

The patient interface 3000 in accordance with one form of the present technology is constructed and arranged to be able to provide a supply of air at a positive pressure of at least 10 cmH2O with respect to ambient.

The patient interface 3000 in accordance with one form of the present technology is constructed and arranged to be able to provide a supply of air at a positive pressure of at least 20 cmH2O with respect to ambient.

### 5.3.1 Seal-Forming Structure

In one form of the present technology, a seal-forming structure 3100 provides a target seal-forming region, and may additionally provide a cushioning function. The target seal-forming region is a region on the seal-forming structure 3100 where sealing may occur. The region where sealing actually occurs—the actual sealing surface—may change within a given treatment session, from day to day, and from patient to patient, depending on a range of factors including for example, where the patient interface was placed on the face, tension in the positioning and stabilising structure and the shape of a patient's face.

In one form the target seal-forming region is located on an outside surface of the seal-forming structure 3100.

In certain forms of the present technology, the seal-forming structure 3100 is constructed from a biocompatible material, e.g. silicone rubber.

A seal-forming structure 3100 in accordance with the present technology may be constructed from a soft, flexible, resilient material such as silicone.

In certain forms of the present technology, a system is provided comprising more than one a seal-forming structure 3100, each being configured to correspond to a different size and/or shape range. For example the system may comprise one form of a seal-forming structure 3100 suitable for a large sized head, but not a small sized head and another suitable for a small sized head, but not a large sized head.

### 5.3.1.1 Sealing Mechanisms

In one form, the seal-forming structure includes a sealing flange utilizing a pressure assisted sealing mechanism. In use, the sealing flange can readily respond to a system positive pressure in the interior of the plenum chamber 3200 acting on its underside to urge it into tight sealing engagement with the face. The pressure assisted mechanism may act in conjunction with elastic tension in the positioning and stabilising structure.

In one form, the seal-forming structure 3100 comprises a sealing flange and a support flange. The sealing flange comprises a relatively thin member with a thickness of less than about 1 mm, for example about 0.25 mm to about 0.45 mm, which extends around the perimeter of the plenum chamber 3200. Support flange may be relatively thicker than the sealing flange. The support flange is disposed between the sealing flange and the marginal edge of the plenum chamber 3200, and extends at least part of the way around the perimeter. The support flange is or includes a spring-like element and functions to support the sealing flange from buckling in use.

In one form, the seal-forming structure may comprise a compression sealing portion or a gasket sealing portion. In use the compression sealing portion, or the gasket sealing portion is constructed and arranged to be in compression, e.g. as a result of elastic tension in the positioning and stabilising structure.

In one form, the seal-forming structure comprises a tension portion. In use, the tension portion is held in tension, e.g. by adjacent regions of the sealing flange.

In one form, the seal-forming structure comprises a region having a tacky or adhesive surface.

In certain forms of the present technology, a seal-forming structure may comprise one or more of a pressure-assisted sealing flange, a compression sealing portion, a gasket sealing portion, a tension portion, and a portion having a tacky or adhesive surface.

### 5.3.1.2 Nose Bridge or Nose Ridge Region

In one form, the non-invasive patient interface 3000 comprises a seal-forming structure that forms a seal in use on a nose bridge region or on a nose-ridge region of the patient's face.

In one form, the seal-forming structure includes a saddle-shaped region constructed to form a seal in use on a nose bridge region or on a nose-ridge region of the patient's face.

### 5.3.1.3 Upper Lip Region

In one form, the non-invasive patient interface 3000 comprises a seal-forming structure that forms a seal in use on an upper lip region (that is, the lip superior) of the patient's face.

In one form, the seal-forming structure includes a saddle-shaped region constructed to form a seal in use on an upper lip region of the patient's face.

### 5.3.1.4 Chin-Region

In one form the non-invasive patient interface 3000 comprises a seal-forming structure that forms a seal in use on a chin-region of the patient's face.

In one form, the seal-forming structure includes a saddle-shaped region constructed to form a seal in use on a chin-region of the patient's face.

### 5.3.1.5 Forehead Region

In one form, the seal-forming structure that forms a seal in use on a forehead region of the patient's face. In such a form, the plenum chamber may cover the eyes in use.

### 5.3.1.6 Nasal Pillows

In one form the seal-forming structure of the non-invasive patient interface 3000 comprises a pair of nasal puffs, or nasal pillows, each nasal puff or nasal pillow being constructed and arranged to form a seal with a respective naris of the nose of a patient.

Nasal pillows in accordance with an aspect of the present technology include: a frusto-cone, at least a portion of which forms a seal on an underside of the patient's nose, a stalk, a flexible region on the underside of the frusto-cone and connecting the frusto-cone to the stalk. In addition, the structure to which the nasal pillow of the present technology is connected includes a flexible region adjacent the base of the stalk. The flexible regions can act in concert to facilitate a universal joint structure that is accommodating of relative movement both displacement and angular of the frusto-cone and the structure to which the nasal pillow is connected. For example, the frusto-cone may be axially displaced towards the structure to which the stalk is connected.

### 5.3.2 Plenum Chamber

The plenum chamber 3200 has a perimeter that is shaped to be complementary to the surface contour of the face of an average person in the region where a seal will form in use. In use, a marginal edge of the plenum chamber 3200 is positioned in close proximity to an adjacent surface of the face. Actual contact with the face is provided by the seal-forming structure 3100. The seal-forming structure 3100 may extend in use about the entire perimeter of the plenum chamber 3200. In some forms, the plenum chamber 3200 and the seal-forming structure 3100 are formed from a single homogeneous piece of material.

In certain forms of the present technology, the plenum chamber 3200 does not cover the eyes of the patient in use. In other words, the eyes are outside the pressurised volume defined by the plenum chamber. Such forms tend to be less obtrusive and/or more comfortable for the wearer, which can improve compliance with therapy.

In certain forms of the present technology, the plenum chamber 3200 is constructed from a transparent material, e.g. a transparent polycarbonate. The use of a transparent material can reduce the obtrusiveness of the patient interface, and help improve compliance with therapy. The use of a transparent material can aid a clinician to observe how the patient interface is located and functioning.

In certain forms of the present technology, the plenum chamber 3200 is constructed from a translucent material. The use of a translucent material can reduce the obtrusiveness of the patient interface, and help improve compliance with therapy.

### 5.3.3 Positioning and Stabilising Structure

The seal-forming structure 3100 of the patient interface 3000 of the present technology may be held in sealing position in use by the positioning and stabilising structure 3300.

In one form the positioning and stabilising structure 3300 provides a retention force at least sufficient to overcome the effect of the positive pressure in the plenum chamber 3200 to lift off the face.

In one form the positioning and stabilising structure 3300 provides a retention force to overcome the effect of the gravitational force on the patient interface 3000.

In one form the positioning and stabilising structure 3300 provides a retention force as a safety margin to overcome the potential effect of disrupting forces on the patient interface 3000, such as from tube drag, or accidental interference with the patient interface.

In one form of the present technology, a positioning and stabilising structure 3300 is provided that is configured in a manner consistent with being worn by a patient while sleeping. In one example the positioning and stabilising structure 3300 has a low profile, or cross-sectional thickness, to reduce the perceived or actual bulk of the apparatus. In one example, the positioning and stabilising structure 3300 comprises at least one strap having a rectangular cross-section. In one example the positioning and stabilising structure 3300 comprises at least one flat strap.

In one form of the present technology, a positioning and stabilising structure 3300 is provided that is configured so as not to be too large and bulky to prevent the patient from lying in a supine sleeping position with a back region of the patient's head on a pillow.

In one form of the present technology, a positioning and stabilising structure 3300 is provided that is configured so as not to be too large and bulky to prevent the patient from lying in a side sleeping position with a side region of the patient's head on a pillow.

In one form of the present technology, a positioning and stabilising structure 3300 is provided with a decoupling portion located between an anterior portion of the positioning and stabilising structure 3300, and a posterior portion of the positioning and stabilising structure 3300. The decoupling portion does not resist compression and may be, e.g. a flexible or floppy strap. The decoupling portion is constructed and arranged so that when the patient lies with their head on a pillow, the presence of the decoupling portion prevents a force on the posterior portion from being transmitted along the positioning and stabilising structure 3300 and disrupting the seal.

In one form of the present technology, a positioning and stabilising structure 3300 comprises a strap constructed from a laminate of a fabric patient-contacting layer, a foam inner layer and a fabric outer layer. In one form, the foam is porous to allow moisture, (e.g., sweat), to pass through the strap. In one form, the fabric outer layer comprises loop material to engage with a hook material portion.

In certain forms of the present technology, a positioning and stabilising structure 3300 comprises a strap that is extensible, e.g. resiliently extensible. For example the strap may be configured in use to be in tension, and to direct a force to draw a seal-forming structure into sealing contact with a portion of a patient's face. In an example the strap may be configured as a tie.

In one form of the present technology, the positioning and stabilising structure comprises a first tie, the first tie being constructed and arranged so that in use at least a portion of an inferior edge thereof passes superior to an otobasion superior of the patient's head and overlays a portion of a parietal bone without overlaying the occipital bone.

In one form of the present technology suitable for a nasal-only mask or for a full-face mask, the positioning and stabilising structure includes a second tie, the second tie being constructed and arranged so that in use at least a portion of a superior edge thereof passes inferior to an otobasion inferior of the patient's head and overlays or lies inferior to the occipital bone of the patient's head.

In one form of the present technology suitable for a nasal-only mask or for a full-face mask, the positioning and stabilising structure includes a third tie that is constructed and arranged to interconnect the first tie and the second tie to reduce a tendency of the first tie and the second tie to move apart from one another.

In certain forms of the present technology, a positioning and stabilising structure 3300 comprises a strap that is bendable and e.g. non-rigid. An advantage of this aspect is that the strap is more comfortable for a patient to lie upon while the patient is sleeping.

In certain forms of the present technology, a positioning and stabilising structure 3300 comprises a strap constructed to be breathable to allow moisture vapour to be transmitted through the strap,

In certain forms of the present technology, a system is provided comprising more than one positioning and stabilizing structure 3300, each being configured to provide a retaining force to correspond to a different size and/or shape range. For example the system may comprise one form of positioning and stabilizing structure 3300 suitable for a large sized head, but not a small sized head, and another. suitable for a small sized head, but not a large sized head.

### 5.3.4 Vent

In one form, the patient interface 3000 includes a vent 3400 constructed and arranged to allow for the washout of exhaled gases, e.g. carbon dioxide.

In certain forms the vent 3400 is configured to allow a continuous vent flow from an interior of the plenum chamber 3200 to ambient whilst the pressure within the plenum chamber is positive with respect to ambient. The vent 3400 is configured such that the vent flow rate has a magnitude sufficient to reduce rebreathing of exhaled CO2 by the patient while maintaining the therapeutic pressure in the plenum chamber in use.

One form of vent 3400 in accordance with the present technology comprises a plurality of holes, for example, about 20 to about 80 holes, or about 40 to about 60 holes, or about 45 to about 55 holes.

The vent 3400 may be located in the plenum chamber 3200. Alternatively, the vent 3400 is located in a decoupling structure, e.g., a swivel.

### 5.3.5 Decoupling Structure(s)

In one form the patient interface 3000 includes at least one decoupling structure, for example, a swivel or a ball and socket.

### 5.3.6 Connection Port

Connection port 3600 allows for connection to the air circuit 4170.

### 5.3.7 Forehead Support

In one form, the patient interface 3000 includes a forehead support 3700.

### 5.3.8 Anti-Asphyxia Valve

In one form, the patient interface 3000 includes an anti-asphyxia valve.

### 5.3.9 Ports

In one form of the present technology, a patient interface 3000 includes one or more ports that allow access to the volume within the plenum chamber 3200. In one form this allows a clinician to supply supplementary oxygen. In one form, this allows for the direct measurement of a property of gases within the plenum chamber 3200, such as the pressure.

### 5.3.10 Self-Contained Unit

As illustrated in FIGS. 7 to 22, some forms of the patient interface may be self-contained units. For example, the patient interfaces may not need to be connected to an external device to receive a flow of pressurized air. Instead, the patient interface itself may include a motor for delivering pressurized airflow directly to a patient.

In some forms, this may enable the patient interface to be more portable, which may be particularly beneficial for patients who travel. For example, the patient may be able to pack a smaller, more portable component. This may promote the continuance of therapy while the patient is away from home.

In some forms, the self-contained patient interface may promote better sleep in the patient or in a bed partner. For example, the patient may not be tethered to an RPT device, which could restrict movement while sleeping. This may allow the patient to roll or otherwise move while sleeping without being constrained. Similarly, the patient's bed partner may experience a better sleep if the patient is able to sleep throughout the night.

In certain forms, a patient (and/or the patient's bed partner) may dislike the intrusiveness of wires, tubes, and/or cords, and may find the medical appearance of the patient interface aesthetically unappealing. This could lead to lower compliance with the therapy. By reducing the external attachments on the self-contained patient interface, a patient may be more likely to use the patient interface. For example, as described below, the material of the patient interface, combined with the lack of external attachments, may reduce the medical feel of the patient interface.

In some forms, providing a single unit may be more intuitive for a patient to use. For example, the patient may need to interact with a single device, which may simplify the steps necessary to learn how to use the device.

As described below, the patient interfaces illustrated in FIGS. 7 to 22 may be similar to the patient interface 3000 described above (see e.g., FIG. 3A), and only some similarities and differences may be described.

### 5.3.10.1 Full-Face Interface

As illustrated in FIGS. 7 to 16, some forms of the patient interface are a full-face patient interface. For example, the patient interface may form a seal around the patient's nares and the patient's mouth so that pressurized air may be delivered to the patient's airways through either the patient's nose and/or the patient's mouth.

As illustrated in FIGS. 7 to 9, a first version of a full-face patient interface 6000 may include a seal-forming structure 6100, a plenum chamber 6200, a positioning and stabilising structure 6300, and a flow generator casing 6400.

The seal-forming structure 6100 may be constructed from a flexible material and may be comfortable when contacting the patient's face. For example, the seal-forming structure 6100 may be formed from a silicone material. Alternatively or additionally, the seal-forming structure 6100 may be formed from a textile material. The seal-forming structure may have a frame made of a more rigid material (e.g., plastic or polycarbonate) than the seal portion, and the frame may be attached to the casing 6408 as described in relation to FIG. 31. The seal portion and/or the frame may have a gas washout vent with one or more holes to exhaust exhaled gas to ambient. Moreover, especially in the case where the patient interface is a full face mask or oro-nasal mask, the seal-forming structure and/or the casing (described below) may include an anti-asphyxia valve (AAV) to allow the patient to breath in ambient air if the patient interface is not connected to a power source (e.g., due to a power outage).

In the illustrated example, the seal-forming structure 6100 may have a substantially low profile. As described above, a patient may feel uncomfortable wearing a large device, and may therefore be dissuaded from continuing the therapy. The seal-forming structure 6100 may therefore minimize the sealing area (e.g., area within the seal-forming structure 6100).

In certain forms, the nasal seal 6104 of the seal-forming structure 6100 may not contact a ridge of the patient's nose. For example, the seal-forming structure 6100 may seal around the patient's alar rims while avoiding contact with the patient's nasal ridge. Patients may find this more comfortable because less of their face is in contact with the seal and subjected to the therapeutic pressure.

In some forms, the seal-forming structure 6100 may not extend substantially beyond the plenum chamber 6200 and/or the flow generator casing 6400. For example, a height of the plenum chamber 6200 and/or the flow generator casing 6400 may be substantially the same as the height of the sealing area (e.g., measured from the patient's lip inferior to the pronasale). In other examples, the plenum chamber 6200 and/or the flow generator casing 6400 may extend inferior to the seal-forming structure 6100 (e.g., toward the supramenton) but may not extend substantially superior to the seal-forming structure (e.g., above the pronasale and onto the patient's nasal ridge). This may assist in contributing to a low profile of the patient interface 6000. For example, the plenum chamber 6200 and/or the flow generator casing 6400 may not substantially obstruct the patient's line of sight.

In some forms, the positioning and stabilising structure 6300 may be connected to the flow generator casing 6400. As described later, the plenum chamber 6200 and the seal-forming structure 6100 are connected to the flow generator casing 6400. The positioning and stabilising structure 6300 may therefore provide a tensile force for maintaining the seal-forming structure 6100 in a sealing position on the patient's face.

The positioning and stabilising structure 6300 may be formed as a headgear and may include a front strap 6304. The front strap 6304 may contact the patient's face between the respective eye and ear and pass over top of the patient's head. In other words, the front strap 6304 may contact the patient's cheeks and may overlay the frontal bone and/or the parietal bone on the patient's head.

In some forms, the front strap 6304 may include ends 6308 that connect to the flow generator casing 6400. In some examples, the ends 6308 may be permanently connected (e.g., via an adhesive, stitching, welding, etc.) to the flow generator casing 6400. In other examples, the ends 6308 may be removably connected (e.g., via a mechanical fastener, hook and look material, magnets, etc.) to the flow generator casing 6400. Although not shown, the ends 6308 may alternatively be connected directly to the plenum chamber 6200 (e.g., either removably or permanently).

In some forms, the front strap 6304 may be constructed from a textile or other comfortable material (e.g., a material that is flexible and soft to the touch). The textile material may promote patient compliance because it more closely resembles bed clothes and not a medical device. The improved comfort as well as the aesthetically pleasing look may encourage patients to continue to wear the patient interface 6000 and continue the therapy.

As illustrated in FIGS. 7 and 8, certain forms of the front strap 6304 may include one or more rigidizers 6312. The rigidizers 6312 may be enclosed by the textile material of the front strap 6304 so that the rigidizer 6312 is not exposed (e.g., FIG. 7 illustrates the outline of the rigidizer 6312 beneath the textile material of the front strap 6304). Encasing the rigidizer 6312 in the textile material may improve patient comfort by limiting patient contact with uncomfortable materials.

In certain forms, a single rigidizer 6312 may extend around the front strap 6304 (e.g., substantially between the ends 6308). The rigidizer 6312 may limit the extension of the top strap 6304 in order to maintain the top strap 6304 (and therefore the seal-forming structure 6100) in a desired position. Alternatively, there may be multiple rigidizers 6312 around the perimeter of the top strap 6304. There may be gaps between the different rigidizers 6312 where localized stretching may occur.

Alternatively or additionally, the top strap 6304 may include padding or a cushioning material. For example, the top strap 6304 may include the padding in place of the rigidizer 6312 or may include the padding in addition to the rigidizer 6312. The padding may be formed from a foam or other compressible material. The padding may provide increased comfort for a side sleeping patient, which may further encourage the continuation of therapy.

As shown in FIGS. 7 and 8, some forms of the front strap 6304 may include a button or user engagement device 6314. The button 6314 may be connected to a latch (not shown). The button 6314 and latch may move together when the patient actuates the button 6314. As described below, the button 6314 may enable a power source to removably connect to the positioning and stabilising structure 6300.

In some forms, the positioning and stabilising structure 6300 may further include an upper back strap 6316. The upper back strap 6316 may contact a posterior portion of the patient's head in use. For example, the upper back strap 6316 may contact the patient's head superior to a respective ear (e.g., overlaying a temporal bone) and extend toward the back of the patient's head (e.g., overlaying the occipital bone).

In some forms, the upper back strap 6316 may connect to the front strap 6304. For example, the upper back strap 6316 may connect to the front strap 6304 at a location superior to the patient's ear in use (e.g., so that the upper back strap 6316 does not intersect with the patient's ear). In some forms, the upper back strap 6316 may be permanently connected to the front strap 6304, while in other examples, the upper back strap 6316 may be removably connected to the front strap 6304.

As illustrated in FIGS. 7 and 8, some forms of the upper back strap 6316 may be constructed from an elastic material. For example, the upper back strap 6316 may be more stretchable or extendible than the front strap 6304. The elasticity of the upper back strap 6316 may allow the positioning and stabilising structure 6300 to fit a variety of patients' heads while also providing a tensile force to maintain the sealing position of the seal-forming structure 6100. The elastic material may also be similarly comfortable as the textile material of the front strap 6304.

In other forms, the upper back strap 6316 may be formed from an inextensible material.

In some forms, the positioning and stabilising structure 6300 may further include a lower back strap 6320. The lower back strap 6320 may contact a posterior portion of the patient's head in use. For example, the lower back strap 6320 may contact the patient's head inferior to a respective ear (e.g., overlaying the masseter muscle) and extend toward the back of the patient's head (e.g., overlaying the occipital bone).

In some forms, the lower back strap 6320 may connect to the front strap 6304. For example, the lower back strap 6320 may connect to the front strap 6304 at a location inferior to the patient's ear in use (e.g., so that the lower back strap 6320 does not intersect with the patient's ear). As illustrated in FIGS. 7 to 9, the lower back strap 6320 may be removably connected to the front strap 6304 (e.g., by a magnet in FIGS. 7 and 8, or by a hook and loop material in FIG. 9). Alternatively, the lower back strap 6320 may be permanently connected to the front strap 6304

As illustrated in FIGS. 7 and 8, some forms of the lower back strap 6320 may be constructed from an elastic material. For example, the lower back strap 6320 may be more stretchable or extendible than the front strap 6304. The upper and lower back straps 6316, 6320 may be constructed from the same material, or each strap may include a different extensibility. The elasticity of the lower back strap 6320 may allow the positioning and stabilising structure 6300 to fit a variety of patients' heads while also providing a tensile force to maintain the sealing position of the seal-forming structure 6100. The elastic material may also be similarly comfortable as the textile material of the front strap 6304.

As illustrated in FIGS. 7 and 8, a connector strap 6324 may connect the upper and lower back straps 6316, 6320. The connector strap 6324 may overlay the patient's occipital bone in use. The connector strap 6324 may limit the relative movement between the upper and lower back straps 6316, 6320.

As shown in FIG. 9, an alternate version of a positioning and stabilising structure 6350 may be used and connected to the flow generator casing 6400. The positioning and stabilising structure 6350 may be a knit headgear (e.g., constructed at least partially from a textile). The positioning and stabilising structure 6350 may be formed as a single piece (e.g., individual straps may not be disconnectable from one another).

In some forms, the positioning and stabilising structure 6350 may contact the patient's head in substantially the same manner (e.g., in substantially the same location) as the positioning and stabilising structure 6300.

In some forms, portions of the positioning and stabilising structure 6350 may include mesh material. The mesh and the textile that form the positioning and stabilising structure 6350 may have different extensibilities, so that the positioning and stabilising structure 6350 may stretch in a predetermined manner.

In some forms, portions of the positioning and stabilising structure 6350 may be rigidized. For example, stitching may be applied to various portions of the positioning and stabilising structure 6350 in order to limit where and how much the positioning and stabilising structure 6350 may stretch,

In certain forms, the stitching may also provide structural rigidity to the positioning and stabilising structure 6350. For example, the positioning and stabilising structure 6350 may be able to maintain a three-dimensional shape when not worn by the patient (e.g., similar to the positioning and stabilising structure 6300 with the rigidizer 6312).

With continued reference to FIG. 9, the positioning and stabilising structure 6350 may not be permanently connected to the flow generator casing 6400 (or alternatively to the plenum chamber). Instead, the positioning and stabilising structure 6350 may be removably connected.

For example, the flow generator casing 6400 may include a strap opening 6402 that a strap of the positioning and stabilising structure 6350 may be threaded through. The patient may adjust a length of the strap that is threaded through in order to control the tensile force provided by the positioning and stabilising structure 6350.

In certain forms, the positioning and stabilising structure 6350 may include hook and loop material (not shown) in order to retain the straps in a desired position.

In some forms, the flow generator casing 6400 in FIGS. 7 to 9 may include a front case 6404 connected to a rear case 6406. The front case 6404 may be connected to the rear case 6406 in order to house electrical components described in detail below.

In some forms, the rear case 6406 may be larger than the front case 6404. The front case 6404 may fit within the rear case 6406 in a substantially flush arrangement.

For example, the front and rear cases 6404, 6406 may each include a curvature that is substantially the same as the other curvature. When the rear case 6406 receives the front case 6404, the curvatures may align so that there is a substantially smooth transition between the front and rear cases 6404, 6406.

In some forms, the rear case 6406 may be covered in a textile material. The rear case 6406 is positioned closer to the patient's face than the front case 6404, and the textile material may assist in improving patient comfort and/or the overall aesthetic of the patient interface 6000.

In some forms, the front case 6404 and/or the rear case 6406 may be constructed in different colors and/or different finishes (e.g., gloss, matte, etc.). Patients may choose the color and/or finish based on person preference in order to increase the overall aesthetic appeal of the patient interface 6000.

In some forms, the front and rear cases 6404, 6406 may be removably connected to one another. For example, the front case 6404 may disconnect from the rear case 6406 in order to expose a cavity 6408 (see e.g., FIG. 31) where electrical components may be housed. The front and rear cases 6404, 6406 may be removably connected with a snap-fit, friction fit, a press fit, and/or a magnetic engagement.

FIGS. 10 to 12 may illustrate an alternate example of the first version of a full-face patient interface 6000. As described below, the full-face patient interface 6000 illustrated in FIGS. 7 to 9 may differ from the full-face patient interface in FIGS. 10 to 12 by the means of electrical power supplied to the patient interface 6000. For example, the patient interface 6000 of FIGS. 7 to 9 uses a power cord 6020 to connect to a battery 6010 separate from the patient interface 6000, while the patient interface 6000 of FIGS. 10 to 12 uses a battery 6030 connected directly to the patient interface 6000.

As shown in FIG. 13, a second version of a full-face patient interface 7000 may include a seal-forming structure 7100, a plenum chamber 7200, a positioning and stabilising structure 7300, and a flow generator casing 7400. The second version of the full-face patient interface 7000 may be similar to the first version of the full-face patient interface 6000. Accordingly, only some similarities and differences may be described below. For example, similar features may include similar reference numbers plus “1000”.

In some forms, the flow generator casing 7400 includes a central section 7430 and a pair of side sections 7432 (one illustrated in FIG. 13). The central section 7430 may be positioned proximate to the plenum chamber 7200 (e.g., aligned with the sagittal plane of the patient in use). The side sections 7432 may be disposed on either lateral side of the central section 7430.

In some forms, the central section 7430 and the side sections 7432 may include each include a curvature. The curvature of each section 7430, 7432 may have substantially the same curvature so that the sections 7430, 7432 form a substantially smooth interface (e.g., without sharp corners).

In certain forms, the curvature of the central and side sections 7430, 7432 may be similar to the curvature of the patient's face (e.g., of the mandible and/or maxilla). The curvature of the flow generator casing 7400 may assist in creating a smaller profile for the patient interface 7000 in order to reduce slight-line obstructions and make the patient interface 7000 more comfortable to wear.

As illustrated in FIG. 13 (as well as in FIG. 15), the central section 7430 may extend more superior than at least a portion of the seal-forming structure 7100 (e.g., more superior than the nasal portion 7104 of the seal-forming structure 7100). For example, a center of the central section 7430 (e.g., intersecting the sagittal plane in use) may be superior to the patient's pronasale in use.

In some forms, the central section 7430 may be removable relative to the side sections 7432. The central section 7430 may be formed from a rigid material (e.g., plastic) and may protect electrical components of the patient interface 7000.

FIGS. 14 to 16 may illustrate an alternate example of the second version of a full-face patient interface 7000. As described below, the full-face patient interface 7000 illustrated in FIG. 13 may differ from the full-face patient interface in FIGS. 14 to 16 by the means of electrical power supplied to the patient interface 7000. For example, the patient interface 7000 of FIG. 13 uses a power cord 6020 to connect to a battery 6010 separate from the patient interface 7000, while the patient interface 7000 of FIGS. 14 to 16 uses a battery 6035 connected directly to the patient interface 7000.

In some forms, the battery 6035 may include a curvature that substantially replicates the curvature of the front strap 7304. This may allow a substantially flush engagement between the battery 6035 and the front strap 7304 (e.g., there may be no sharp corners between the battery 6035 and the front strap 7304). Additionally, the battery 6035 and the front strap 7304 may form a substantially smooth curvature between the ends 7308. This may create a smaller profile (e.g., as compared to the first version illustrated in FIGS. 10 to 12). The smaller profile may provide a more aesthetically pleasing appearance and may maintain the patient interface's center of gravity closer to the patient's head.

### 5.3.10.2 Nasal Interface

As illustrated in FIGS. 17 to 22, some forms of the patient interface are a nasal patient interface. For example, the patient interface may form a seal around the patient's nares so that pressurized air may be delivered to the patient's airways through the patient's nose. A seal may not be formed around the patient's mouth so that the patient's mouth remains exposed to ambient pressure.

As illustrated in FIG. 17, a first version of a nasal patient interface 8000 may include a seal-forming structure 8100, a plenum chamber 8200, a positioning and stabilising structure 8300, and a flow generator casing 8400.

The seal-forming structure 8100 may be constructed from a flexible material and may be comfortable when contacting the patient's face. For example, the seal-forming structure 8100 may be formed from a silicone material. Alternatively or additionally, the seal-forming structure 8100 may be formed from a textile material.

In the illustrated example, the seal-forming structure 8100 may have a substantially low profile. As described above, a patient may feel uncomfortable wearing a large device, and may therefore be dissuaded from continuing the therapy. The seal-forming structure 8100 may therefore minimize the sealing area (e.g., area within the seal-forming structure 8100).

In certain forms, the nasal seal 8104 of the seal-forming structure 8100 may not contact a ridge of the patient's nose. For example, the seal-forming structure 6100 may seal around the patient's alar rims while avoiding contact with the patient's nasal ridge. Patient's may find this more comfortable because less of their face is in contact with the seal and subjected to the therapeutic pressure.

In some forms, the seal-forming structure 8100 may not extend substantially beyond the plenum chamber 8200 and/or the flow generator casing 8400. For example, a height of the plenum chamber 8200 and/or the flow generator casing 8400 may be substantially the same as the height of the sealing area (e.g., measured from the patient's lip inferior to the pronasale). In other examples, the plenum chamber 8200 and/or the flow generator casing 8400 may extend inferior to the seal-forming structure 8100 (e.g., toward the supramenton) but may not extend substantially superior to the seal-forming structure (e.g., above the pronasale and onto the patient's nasal ridge). This may assist in contributing to a low profile of the patient interface 8000. For example, the plenum chamber 8200 and/or the flow generator casing 8400 may not substantially obstruct the patient's line of sight. Moreover, there is no tube and no forehead support that extend above between the patient's eyes.

In some forms, the positioning and stabilising structure 8300 may be connected to the flow generator casing 8400. As described later, the plenum chamber 8200 and the seal-forming structure 8100 are connected to the flow generator casing 8400. The positioning and stabilising structure 8300 may therefore provide a tensile force for maintaining the seal-forming structure 8100 in a sealing position on the patient's face.

The positioning and stabilising structure 8300 may be formed as a headgear and may include a front strap 8304. The front strap 8304 may contact the patient's face between the respective eye and ear and pass over top of the patient's head. In other words, the front strap 8304 may contact the patient's cheeks and may overlay the frontal bone and/or the parietal bone on the patient's head.

In some forms, the front strap 8304 may include ends 8308 that connect to the flow generator casing 8400. In some examples, the ends 8308 may be permanently connected (e.g., via an adhesive, stitching, welding, etc.) to the flow generator casing 8400. In other examples, the ends 8308 may be removably connected (e.g., via a press fit, snap fit, frictional fit, etc.) to the flow generator casing 8400. Although not shown, the ends 8308 may alternatively be connected directly to the plenum chamber 8200 (e.g., either removably or permanently).

In certain forms, the flow generator casing 8400 may extend beyond the seal-forming structure 8100 (e.g., in the posterior direction, in use). Ends 8450 of the flow generator casing 8400 may form a substantially flush connection with the ends 8308 of the front strap 8304. The ends 8450 of the flow generator casing 8400 may therefore form part of the positioning and stabilising structure 8300 once connected to the front strap 8304.

In some forms, the front strap 8304 may be constructed from a textile or other comfortable material (e.g., a material that is flexible and soft to the touch). The textile material may promote patient compliance because it more closely resembles bed clothes and not a medical device. The improved comfort as well as the aesthetically pleasing look may encourage patients to continue to wear the patient interface 8000 and continue the therapy.

In some forms, the front strap 8304 may include one or more rigidizing elements. As illustrated in FIG. 17, certain forms of the front strap 8304 may include one or more rigidizers (not shown). The rigidizers may be enclosed by the textile material of the front strap 8304 so that the rigidizer is not exposed (e.g., FIG. 17 illustrates a rounded shape of the front strap 8304 that may be maintained by the rigidizer beneath the textile material of the front strap 8304). Encasing the rigidizer in the textile material may improve patient comfort by limiting patient contact with uncomfortable materials.

In certain forms, a single rigidizer 6312 may extend around the front strap 6304 (e.g., substantially between the ends 6308). The rigidizer 6312 may limit the extension of the top strap 6304 in order to maintain the top strap 6304 (and therefore the seal-forming structure 6100) in a desired position. Alternatively, there may be multiple rigidizers 6312 around the perimeter of the top strap 6304. There may be gaps between the different rigidizers 6312 where localized stretching may occur.

Alternatively or additionally, the top strap 8304 may include padding or a cushioning material. For example, the top strap 8304 may include the padding in place of the rigidizer or may include the padding in addition to the rigidizer. The padding may also assist in providing a three-dimensional shape for the front strap 8304. The padding may be formed from a foam or other compressible material. The padding may provide increased comfort for a side sleeping patient, which may further encourage the continuation of therapy.

In some forms, the positioning and stabilising structure 8300 may further include an upper back strap 8316. The upper back strap 8316 may contact a posterior portion of the patient's head in use. For example, the upper back strap 8316 may contact the patient's head superior to a respective ear (e.g., overlaying a temporal bone) and extend toward the back of the patient's head (e.g., overlaying the occipital bone).

In some forms, the upper back strap 8316 may connect to the front strap 8304. For example, the upper back strap 8316 may connect to the front strap 8304 at a location superior to the patient's ear in use (e.g., so that the upper back strap 8316 does not intersect with the patient's ear). In some forms, the upper back strap 8316 may be permanently connected to the front strap 8304, while in other examples, the upper back strap 8316 may be removably connected to the front strap 8304.

As illustrated in FIG. 17, some forms of the upper back strap 8316 may be constructed from an elastic material. For example, the upper back strap 8316 may be more stretchable or extendible than the front strap 8304. The elasticity of the upper back strap 8316 may allow the positioning and stabilising structure 8300 to fit a variety of patients' heads while also providing a tensile force to maintain the sealing position of the seal-forming structure 8100. The elastic material may also be similarly comfortable as the textile material of the front strap 8304.

In other forms, the upper back strap 8316 may be formed from an inextensible material.

In some forms, the flow generator casing 8400 in FIG. 17 may include a front case 8404 connected to a rear case 8406. The front case 8404 may be connected to the rear case 8406 in order to house electrical components described in detail below.

In some forms, the rear case 8406 may be larger than the front case 8404. The front case 8404 may fit within the rear case 8406 in a substantially flush arrangement.

For example, the front and rear cases 8404, 8406 may each include a curvature that is substantially the same as the other curvature. When the rear case 8406 receives the front case 8404, the curvatures may align so that there is a substantially smooth transition between the front and rear cases 8404, 8406.

In some forms, the rear case 8406 may be covered in a textile material. The rear case 8406 is positioned closer to the patient's face than the front case 8404, and the textile material may assist in improving patient comfort and/or the overall aesthetic of the patient interface 8000.

In some forms, the front case 8404 and/or the rear case 8406 may be constructed in different colors and/or different finishes (e.g., gloss, matte, etc.). Patients may choose the color and/or finish based on person preference in order to increase the overall aesthetic appeal of the patient interface 8000.

In some forms, the rear case 8406 may project a distance away from the plenum chamber 8200. For example, the distance between the front case 8404 and the plenum chamber 8200 may be smaller than the similar distance measured in the full-face patient interfaces 6000, 7000 described above. The rear case 8406 may be smaller than the rear case 6406. The extra distance extending from the patient's face may provide additional room to fit electrical components of the patient interface.

In some forms, the casing, e.g., a superior surface 8452 of the casing, e.g., rear case 8406 and/or front case 8404, may include at least one air opening 8454 that may provide fluid communication between the cavity within the flow generator casing 8400 and the ambient. As illustrated in FIG. 17, the superior surface 8452 may include a single opening 8454, which may be formed as a slot extending across the superior surface 8542. The opening 8454 may be an inlet for the flow generator (described below). For example, the flow generator may draw ambient air in through the opening 8454 to then pressurize and distribute to the patient. The opening 8454 may be a slot or space that remains or is formed between the front and rear cases when they are connected to each other.

In some forms, the front and rear cases 8404, 8406 may be removably connected to one another. For example, the front case 8404 may disconnect from the rear case 8406 in order to expose a cavity 6408 (see e.g., FIG. 31) where electrical components may be housed. The front and rear cases 8404, 8406 may be removably connected with a snap-fit, friction fit, a press fit, and/or a magnetic engagement.

FIGS. 18 and 19 may illustrate an alternate example of the first version of a nasal patient interface 8000. As described below, the nasal patient interface 8000 illustrated in FIG. 17 may differ from the nasal patient interface in FIGS. 18 and 19 by the means of electrical power supplied to the patient interface 8000. For example, the patient interface 8000 of FIG. 17 uses a power cord 6020 to connect to a battery 6010 (see e.g., FIG. 33) separate from the patient interface 8000, while the patient interface 8000 of FIGS. 18 and 19 uses a battery 6035 connected directly to the patient interface 8000.

In some forms, the battery 6035 may include a curvature that substantially replicates the curvature of the front strap 9304. This may allow a substantially flush engagement between the battery 6035 and the front strap 9304 (e.g., there may be no sharp corners between the battery 6035 and the front strap 9304). Additionally, the battery 6035 and the front strap 9304 may form a substantially smooth curvature between the ends 9308. This may create a smaller profile (e.g., as compared to the first version illustrated in FIGS. 10 to 12). The smaller profile may provide a more aesthetically pleasing appearance and may maintain the patient interface's center of gravity closer to the patient's head.

As shown in FIG. 20, a second version of a nasal patient interface 9000 may include a seal-forming structure 9100, a plenum chamber 9200, a positioning and stabilising structure 9300, and a flow generator casing 9400. The second version of the nasal patient interface 9000 may be similar to the first version of the nasal patient interface 8000. Accordingly, only some similarities and differences may be described below. For example, similar features may include similar reference numbers plus “1000”.

In some forms, the flow generator casing 9400 may include a front case 9404 and a rear case 9406. The front case 9404 may be connected (e.g., removably or permanently) to the rear case 9406 in order to enclose a cavity (not shown).

In some forms, the front case 9404 and the rear case 9406 may be approximately the same height. In other words, the front case 9404 may not be received within the rear case 9406 like in the first version of the nasal patient interface 8000 (see e.g., FIGS. 17 to 19). Instead, outer edges of the front case 9404 may interface with the outer edges of the rear case 9406. This interface may be substantially smooth in order to limit patient discomfort from sharp edges.

In some forms, a gap or opening 9454 may be formed in the flow generator casing. This may be similar to the opening 8454, and may act as an inlet for the flow generator. However, the opening 9454 may be formed between the front case 9404 and the rear case 9406 instead on in a surface of the front or rear cases 9404, 9406. Other examples may include (either instead of or in addition to the gap between the cases 9404, 9406) at least one opening in the surface of either the front case 9404 or the rear case 9406.

FIGS. 21 and 22 may illustrate an alternate example of the second version of a full-face patient interface 9000. As described below, the full-face patient interface 9000 illustrated in FIG. 20 may differ from the full-face patient interface in FIGS. 21 and 22 by the means of electrical power supplied to the patient interface 9000. For example, the patient interface 9000 of FIG. 20 uses a power cord 6020 to connect to a battery 6010 separate from the patient interface 9000, while the patient interface 9000 of FIGS. 21 and 22 uses a battery 6035 connected directly to the patient interface 9000.

In some forms, the battery 6035 may include a curvature that substantially replicates the curvature of the front strap 9304. This may allow a substantially flush engagement between the battery 6035 and the front strap 9304 (e.g., there may be no sharp corners between the battery 6035 and the front strap 9304). Additionally, the battery 6035 and the front strap 9304 may form a substantially smooth curvature between the ends 9308. This may create a smaller profile (e.g., as compared to the first version illustrated in FIGS. 10 to 12). The smaller profile may provide a more aesthetically pleasing appearance and may maintain the patient interface's center of gravity closer to the patient's head.

The patient interfaces 8000, 9000 may include a smaller flow generator casing 8400, 9400 than the flow generator casings 6400, 7400. In some forms (not shown), the nasal patient interfaces 8000, 9000 may additionally include a mouth seal while retaining the smaller flow generator casing 8400, 9400. A smaller blower may be included in the smaller flow generator casing 8400, 9400, which may provide less noise disturbance to a patient or bed partner.

### 5.3.10.3 Power Source

As described above, a patient wearing a patient interface may feel uncomfortable surrounded by cables or wires. The cables or wires may constrain the patient's movement while wearing the device. The wires and cables may also give the patient interface a medical feel that may contribute to decreased compliance.

However, the wires and cables generally connect the patient interface to an RPT device, which requires electrical power to supply a flow of pressurized air. The self-contained patient interfaces described above include a flow generator (e.g., within the flow generator casing 6400), but still require a power source in order to operate.

The illustrated examples are therefore capable of providing power to the patient interface without substantially diminishing the aesthetically pleasing elements for promoting compliance described above.

### 5.3.10.3.1 Remote Power Source

As illustrated in FIG. 23, a battery 6010 may be provided for providing power to an embodiment of the patient interface. The battery 6010 may store an electrical charge, which may be used to power electrical elements of the patient interface (e.g., the flow generator, sensors, etc.).

In some forms, the battery 6010 is a rechargeable battery and may be reused numerous times. In other forms, the battery 6010 is a single use battery and must be replaced after a predetermined number of usage hours.

As illustrated in FIGS. 8, 9, 17, and 20, a power cord 6020 may be connected to the respective patient interface. Another end of the power cord (not shown) may be connected to the battery 6010. The power cord may transfer electrical energy from the battery 6010 to the electrical components of the patient interface.

In some forms, this may be beneficial for a patient because the patient interface may be used any distance from a wall outlet. Although a cord 6020 still extends from the patient interface, the patient still may experience a greater degree of freedom, both to move and in the location of use.

As illustrated in FIGS. 8, 17, and 20, the power cord 6020 may be connected to the front strap 6304. For example, a socket (not shown) may removably receive the power cord 6020. The socket may be positioned at a superior portion of the front strap 6304 (e.g., proximate to the patient's sagittal plane when worn).

In some forms, electrical wires (not shown) may be contained within the textile material of the front strap 6304. The electrical wires may be in electrical communication with the socket and the electrical components in the flow generator casing 6400. Electrical energy may therefore be transferred between the battery and the electrical components.

In certain forms, the front strap 6304 may include the rigidizer(s) 6312 in order to limit the extension of the electrical wires. For example, the wires extend within the front strap and may not be able to substantially extend without failing. The rigidizer(s) 6312 may limit the extension of the front strap 6304 in order to protect the electrical wires.

As illustrated in FIG. 9, an alternate version includes a socket (not shown) on the flow generator casing 6400, and the power cord 6020 removably connected to the socket. In this form, electrical wires may not be needed through the front strap 6304 as the electrical power from the battery 6010 is delivered directly to the flow generator casing 6400. However, electrical wires may still be present if electrical components (e.g., sensors) are connected to the front strap 6304.

In some forms, the patient interface may include a socket on both the front strap 6304 and the flow generator casing 6400. The patient may connect the power cord 6020 to either socket based on patient comfort and/or patient preference.

### 5.3.10.3.2 Connected Power Source

As illustrated in FIGS. 10 to 12, 14 to 16, 18, 19, 21, and 22, a battery 6030 may be provided for providing power to an embodiment of the patient interface. The battery 6030 may store an electrical charge, which may be used to power electrical elements of the patient interface (e.g., the flow generator, sensors, etc.). An electrical conductor, e.g. wire, may extend from the battery, along a strap of the positioning and stabilising structure, to the blower.

In some forms, the battery 6030 is a rechargeable battery and may be reused numerous times. In other forms, the battery 6030 is a single use battery and must be replaced after a predetermined number of usage hours.

Unlike the battery 6010, the battery 6030 may be connected directly to the respective patient interface. In other words, a power cord may not be required to connect the battery to the patient interface in order to power the various electrical components.

In the illustrated examples, the superior region of the front strap 6304 (e.g., the portion overlaying the frontal bone and/or the parietal bone) may include a battery dock 6328. The battery dock 6328 may have a complementary shape to the battery 6030 so that the battery 6030 may be removably received on the battery dock 6328.

In some forms, the button 6314 may be positioned proximate to the battery dock 6328. For example, the battery 6030 may engage the projection when positioned on the battery dock 6328. The projection may engage the battery 6030 and retain it in position. The button 6314 may be actuated in order to move the projection relative to the battery 6030, so that the battery 6030 may be removed from the battery dock 6328 (e.g., in order to be recharged and/or replaced).

FIGS. 14 to 16 may illustrate an alternate battery 6035 that is connected to the front strap 7304 of the patient interface 7000. The battery 6035 may include the buttons 6037, which are selectively actuatable. A latch (not shown) may be connected to either button 6037 so that movement of the button 6037 moves the latch. The latch may selectively engage the front strap 7304 (e.g., proximate to a battery dock 7328 illustrated in FIG. 13) so that the battery 6035 may be selectively removed from the patient interface 7000. FIGS. 19, 20, 21, and 22 similarly illustrate the alternate battery 6035 with the buttons 6037.

In some forms, the battery 6030 may be covered or encased in a textile material (e.g., the same or similar material as the front strap 6304). This may give the patient interface with the battery 6030 a similar non-medical feel as the positioning and stabilising structure.

In some forms, the battery 6030 may be formed with curved or rounded edges and/or sides. This may reduce sharp edges that may otherwise cause discomfort in the patient. Additionally, the curvature of the battery 6030 may form a smooth (or relatively smooth) interface between the battery 6030 and the front strap 6030. This may assist in providing a low profile design, which may be more comfortable and aesthetically pleasing to the patient.

In some forms, the patient interface may include at least one socket and a battery dock 6328. In other words, the patient may power the patient interface using either the battery 6010 and the power cord 6020, or the battery 6030 attached to the battery dock 6328. The patient may use either battery 6010, 6030 based on patient preference.

### 5.3.10.3.3 Charging Dock

As illustrated in FIGS. 24 and 25, a patient interface may be connected to a charger 6040 in order to recharge the battery 6030. FIGS. 24 and 25 illustrate the full-face patient interface 6000, but any of the patient interfaces (e.g., patient interfaces 7000, 8000, 9000) may be used with the charger 6040. Additionally, either battery (e.g., battery 6030 or battery 6035) may be connected to a patient interface and recharged using the charger 6040.

In some forms, the charger 6040 may include a base 6042 and a charging dock 6044 extending vertically from the charging base 6042. The charging dock 6044 may include a grooved surface that removably receives the patient interface (e.g., the front strap 6304 of the positioning and stabilising structure 6300). The grooved surface may include an electrical connector (not shown) that forms an electrical connection with the patient interface 6000 when received in the charging dock 6044.

For example, the front strap 6304 may be positioned in the grooved surface of the charging dock 6044. The front strap 6304 may be positioned so that the battery dock 6328 of the front strap 6304 is positioned proximate to the grooved surface. In some forms, an inner surface of the front strap 6304 (i.e., portion that contacts the patient) may include an electrical terminal (not shown). The electrical terminal may provide an electrical connection to the battery 6030 connected to the battery dock 6328.

In some forms, the charging dock 6044 may itself be a battery with a stored electrical charge. This may allow the charger 6040 to be freely movable and positionable in a variety of locations in order to charge the battery 6030.

Alternatively or additionally, the charger 6040 may include a power cord (not shown) that may be connected to a wall socket. The wall socket may provide electrical power to the charger 6040, which may in turn provide electrical power to recharge the battery 6030.

In some forms, the positioning and stabilising structure 6300 may substantially maintain its three-dimensional shape when not in use (i.e., on the patient's head). For example, the rigidizer 6312 may maintain the front strap 6304 in a three-dimensional shape (e.g., similar to the shape of the patient's head). The charging dock 6044 may be formed with a height that is substantially same as a distance between the flow generator casing 6400 and the battery dock 6328. This may also be the distance between the patient's mouth and the top of the patient's head measured in the superior direction along the sagittal plane. When connected to the charger 6040 flow generator casing 6400 may be in contact with the charging base 6042 so that it is supported (e.g., and not hanging above the surface). Additionally, the front strap 6304 may not be bunched or folded (e.g., as a result of the height of the charging dock 6044 being less than the distance between the flow generator casing 6400 and the battery dock 6328).

In other forms where the front strap 6304 does not include a rigidizer 6312, the height of the charging dock 6044 may be similar to the height described above so that the flow generator casing 6400 may still contact the charging base 6042.

### 5.3.10.4 Carry Case

FIG. 26 illustrates a case 6050 for transporting the patient interface (e.g., the patient interface 6000 is illustrated but any may be inserted into the case 6050). As described above, the positioning and stabilising structure 6300 may include a three-dimensional shape (e.g., as a result of rigidizers 6312 in the front strap 6304). The case 6050 may be large enough in order to receive the patient interface 6000 without substantially bending the front strap 6304. This may be useful in maintaining the longevity of the positioning and stabilising structure 6300. The back straps (e.g., the upper back strap 6316 and the lower back strap 6320) may not include a rigid element and may be bendable when inserted into the case 6050.

### 5.4 RPT Device

An RPT device 4000 in accordance with one aspect of the present technology comprises mechanical, pneumatic, and/or electrical components and is configured to execute one or more algorithms 4300, such as any of the methods, in whole or in part, described herein. The RPT device 4000 may be configured to generate a flow of air for delivery to a patient's airways, such as to treat one or more of the respiratory conditions described elsewhere in the present document.

In one form, the RPT device 4000 is constructed and arranged to be capable of delivering a flow of air in a range of −20 L/min to +150 L/min while maintaining a positive pressure of at least 4 cmH2O, or at least 10 cmH2O, or at least 20 cmH2O.

The RPT device may have an external housing 4010, formed in two parts, an upper portion 4012 and a lower portion 4014. Furthermore, the external housing 4010 may include one or more panel(s) 4015. The RPT device 4000 comprises a chassis 4016 that supports one or more internal components of the RPT device 4000. The RPT device 4000 may include a handle 4018.

The pneumatic path of the RPT device 4000 may comprise one or more air path items, e.g., an inlet air filter 4112, an inlet muffler 4122, a pressure generator 4140 capable of supplying air at positive pressure (e.g., a blower 4142), an outlet muffler 4124 and one or more transducers 4270, such as pressure sensors 4272 and flow rate sensors 4274.

One or more of the air path items may be located within a removable unitary structure which will be referred to as a pneumatic block 4020. The pneumatic block 4020 may be located within the external housing 4010. In one form a pneumatic block 4020 is supported by, or formed as part of the chassis 4016.

The RPT device 4000 may have an electrical power supply 4210, one or more input devices 4220, a central controller 4230, a therapy device controller 4240, a pressure generator 4140, one or more protection circuits 4250, memory 4260, transducers 4270, data communication interface 4280 and one or more output devices 4290. Electrical components 4200 may be mounted on a single Printed Circuit Board Assembly (PCBA) 4202. In an alternative form, the RPT device 4000 may include more than one PCBA 4202.

### 5.4.1 RPT Device Mechanical & Pneumatic Components

An RPT device may comprise one or more of the following components in an integral unit. In an alternative form, one or more of the following components may be located as respective separate units.

### 5.4.1.1 Air Filter(s)

An RPT device in accordance with one form of the present technology may include an air filter 4110, or a plurality of air filters 4110.

In one form, an inlet air filter 4112 is located at the beginning of the pneumatic path upstream of a pressure generator 4140.

In one form, an outlet air filter 4114, for example an antibacterial filter, is located between an outlet of the pneumatic block 4020 and a patient interface 3000.

### 5.4.1.2 Muffler(s)

An RPT device in accordance with one form of the present technology may include a muffler 4120, or a plurality of mufflers 4120.

In one form of the present technology, an inlet muffler 4122 is located in the pneumatic path upstream of a pressure generator 4140.

In one form of the present technology, an outlet muffler 4124 is located in the pneumatic path between the pressure generator 4140 and a patient interface 3000.

### 5.4.1.3 Pressure Generator

In one form of the present technology, a pressure generator 4140 for producing a flow, or a supply, of air at positive pressure is a controllable blower 4142. For example, the blower 4142 may include a brushless DC motor 4144 with one or more impellers. The impellers may be located in a volute. The blower may be capable of delivering a supply of air, for example at a rate of up to about 120 litres/minute, at a positive pressure in a range from about 4 cmH2O to about 20 cmH2O, or in other forms up to about 30 cmH2O when delivering respiratory pressure therapy. The blower may be as described in any one of the following patents or patent applications the contents of which are incorporated herein by reference in their entirety: U.S. Pat. Nos. 7,866,944; 8,638,014; 8,636,479; and PCT Patent Application Publication No. WO 2013/020167.

The pressure generator 4140 may be under the control of the therapy device controller 4240.

In other forms, a pressure generator 4140 may be a piston-driven pump, a pressure regulator connected to a high pressure source (e.g. compressed air reservoir), or a bellows. 5.4.1.4 Transducer(s)

Transducers may be internal of the RPT device, or external of the RPT device. External transducers may be located for example on or form part of the air circuit, e.g., the patient interface. External transducers may be in the form of non-contact sensors such as a Doppler radar movement sensor that transmit or transfer data to the RPT device.

In one form of the present technology, one or more transducers 4270 are located upstream and/or downstream of the pressure generator 4140. The one or more transducers 4270 may be constructed and arranged to generate signals representing properties of the flow of air such as a flow rate, a pressure or a temperature at that point in the pneumatic path.

In one form of the present technology, one or more transducers 4270 may be located proximate to the patient interface 3000.

In one form, a signal from a transducer 4270 may be filtered, such as by low-pass, high-pass or band-pass filtering.

### 5.4.1.4.1 Flow Rate Sensor

A flow rate sensor 4274 in accordance with the present technology may be based on a differential pressure transducer, for example, an SDP600 Series differential pressure transducer from SENSIRION.

In one form, a signal generated by the flow rate sensor 4274 and representing a flow rate is received by the central controller 4230.

### 5.4.1.4.2 Pressure Sensor

A pressure sensor 4272 in accordance with the present technology is located in fluid communication with the pneumatic path. An example of a suitable pressure sensor is a transducer from the HONEYWELL ASDX series. An alternative suitable pressure sensor is a transducer from the NPA Series from GENERAL ELECTRIC.

In one form, a signal generated by the pressure sensor 4272 and representing a pressure is received by the central controller 4230.

### 5.4.1.4.3 Motor Speed Transducer

In one form of the present technology a motor speed transducer 4276 is used to determine a rotational velocity of the motor 4144 and/or the blower 4142. A motor speed signal from the motor speed transducer 4276 may be provided to the therapy device controller 4240. The motor speed transducer 4276 may, for example, be a speed sensor, such as a Hall effect sensor.

### 5.4.1.5 Anti-Spill Back Valve

In one form of the present technology, an anti-spill back valve 4160 is located between the humidifier 5000 and the pneumatic block 4020. The anti-spill back valve is constructed and arranged to reduce the risk that water will flow upstream from the humidifier 5000, for example to the motor 4144.

### 5.4.2 RPT Device Electrical Components

### 5.4.2.1 Power Supply

A power supply 4210 may be located internal or external of the external housing 4010 of the RPT device 4000.

In one form of the present technology, power supply 4210 provides electrical power to the RPT device 4000 only. In another form of the present technology, power supply 4210 provides electrical power to both RPT device 4000 and humidifier 5000.

### 5.4.2.2 Input Devices

In one form of the present technology, an RPT device 4000 includes one or more input devices 4220 in the form of buttons, switches or dials to allow a person to interact with the device. The buttons, switches or dials may be physical devices, or software devices accessible via a touch screen. The buttons, switches or dials may, in one form, be physically connected to the external housing 4010, or may, in another form, be in wireless communication with a receiver that is in electrical connection to the central controller 4230.

In one form, the input device 4220 may be constructed and arranged to allow a person to select a value and/or a menu option.

### 5.4.2.3 Central Controller

In one form of the present technology, the central controller 4230 is one or a plurality of processors suitable to control an RPT device 4000.

Suitable processors may include an x86 INTEL processor, a processor based on ARM® Cortex®-M processor from ARM Holdings such as an STM32 series microcontroller from ST MICROELECTRONIC. In certain alternative forms of the present technology, a 32-bit RISC CPU, such as an STR9 series microcontroller from ST MICROELECTRONICS or a 16-bit RISC CPU such as a processor from the MSP430 family of microcontrollers, manufactured by TEXAS INSTRUMENTS may also be suitable.

In one form of the present technology, the central controller 4230 is a dedicated electronic circuit.

In one form, the central controller 4230 is an application-specific integrated circuit. In another form, the central controller 4230 comprises discrete electronic components.

The central controller 4230 may be configured to receive input signal(s) from one or more transducers 4270, one or more input devices 4220, and the humidifier 5000.

The central controller 4230 may be configured to provide output signal(s) to one or more of an output device 4290, a therapy device controller 4240, a data communication interface 4280, and the humidifier 5000.

In some forms of the present technology, the central controller 4230 is configured to implement the one or more methodologies described herein, such as the one or more algorithms 4300 which may be implemented with processor-control instructions, expressed as computer programs stored in a non-transitory computer readable storage medium, such as memory 4260. In some forms of the present technology, the central controller 4230 may be integrated with an RPT device 4000. However, in some forms of the present technology, some methodologies may be performed by a remotely located device. For example, the remotely located device may determine control settings for a ventilator or detect respiratory related events by analysis of stored data such as from any of the sensors described herein.

### 5.4.2.4 Clock

The RPT device 4000 may include a clock 4232 that is connected to the central controller 4230.

### 5.4.2.5 Therapy Device Controller

In one form of the present technology, therapy device controller 4240 is a therapy control module 4330 that forms part of the algorithms 4300 executed by the central controller 4230.

In one form of the present technology, therapy device controller 4240 is a dedicated motor control integrated circuit. For example, in one form a MC33035 brushless DC motor controller, manufactured by ONSEMI is used.

### 5.4.2.6 Protection Circuits

The one or more protection circuits 4250 in accordance with the present technology may comprise an electrical protection circuit, a temperature and/or pressure safety circuit.

### 5.4.2.7 Memory

In accordance with one form of the present technology the RPT device 4000 includes memory 4260, e.g., non-volatile memory. In some forms, memory 4260 may include battery powered static RAM. In some forms, memory 4260 may include volatile RAM.

Memory 4260 may be located on the PCBA 4202. Memory 4260 may be in the form of EEPROM, or NAND flash.

Additionally, or alternatively, RPT device 4000 includes a removable form of memory 4260, for example a memory card made in accordance with the Secure Digital (SD) standard.

In one form of the present technology, the memory 4260 acts as a non-transitory computer readable storage medium on which is stored computer program instructions expressing the one or more methodologies described herein, such as the one or more algorithms 4300.

### 5.4.2.8 Data Communication Systems

In one form of the present technology (see e.g., FIG. 4C), a data communication interface 4280 is provided, and is connected to the central controller 4230. Data communication interface 4280 may be connectable to a remote external communication network 4282 and/or a local external communication network 4284. The remote external communication network 4282 may be connectable to a remote external device 4286. The local external communication network 4284 may be connectable to a local external device 4288.

In one form, data communication interface 4280 is part of the central controller 4230. In another form, data communication interface 4280 is separate from the central controller 4230, and may comprise an integrated circuit or a processor.

In one form, remote external communication network 4282 is the Internet. The data communication interface 4280 may use wired communication (e.g. via Ethernet, or optical fibre) or a wireless protocol (e.g. CDMA, GSM, LTE) to connect to the Internet.

In one form, local external communication network 4284 utilises one or more communication standards, such as Bluetooth, or a consumer infrared protocol.

In one form, remote external device 4286 is one or more computers, for example a cluster of networked computers. In one form, remote external device 4286 may be virtual computers, rather than physical computers. In either case, such a remote external device 4286 may be accessible to an appropriately authorised person such as a clinician.

The local external device 4288 may be a personal computer, mobile phone, tablet or remote control.

### 5.4.2.9 Output Devices Including Optional Display, Alarms

As shown in FIG. 4C, an output device 4290 in accordance with the present technology may take the form of one or more of a visual, audio and haptic unit. A visual display may be a Liquid Crystal Display (LCD) or Light Emitting Diode (LED) display.

### 5.4.2.9.1 Display Driver

As shown in FIG. 4C, a display driver 4292 receives as an input the characters, symbols, or images intended for display on the display 4294, and converts them to commands that cause the display 4294 to display those characters, symbols, or images.

### 5.4.2.9.2 Display

As shown in FIG. 4C, a display 4294 is configured to visually display characters, symbols, or images in response to commands received from the display driver 4292. For example, the display 4294 may be an eight-segment display, in which case the display driver 4292 converts each character or symbol, such as the figure “0”, to eight logical signals indicating whether the eight respective segments are to be activated to display a particular character or symbol.

### 5.4.3 RPT Device Algorithms

As mentioned above, in some forms of the present technology (see e.g., FIG. 4D), the central controller 4230 may be configured to implement one or more algorithms 4300 expressed as computer programs stored in a non-transitory computer readable storage medium, such as memory 4260. The algorithms 4300 are generally grouped into groups referred to as modules.

In other forms of the present technology, some portion or all of the algorithms 4300 may be implemented by a controller of an external device such as the local external device 4288 or the remote external device 4286. In such forms, data representing the input signals and/or intermediate algorithm outputs necessary for the portion of the algorithms 4300 to be executed at the external device may be communicated to the external device via the local external communication network 4284 or the remote external communication network 4282. In such forms, the portion of the algorithms 4300 to be executed at the external device may be expressed as computer programs, such as with processor control instructions to be executed by one or more processor(s), stored in a non-transitory computer readable storage medium accessible to the controller of the external device. Such programs configure the controller of the external device to execute the portion of the algorithms 4300.

In such forms, the therapy parameters generated by the external device via the therapy engine module 4320 (if such forms part of the portion of the algorithms 4300 executed by the external device) may be communicated to the central controller 4230 to be passed to the therapy control module 4330.

### 5.4.3.1 Pre-Processing Module

A pre-processing module 4310 in accordance with one form of the present technology (see e.g., FIG. 4D) receives as an input a signal from a transducer 4270, for example a flow rate sensor 4274 or pressure sensor 4272, and performs one or more process steps to calculate one or more output values that will be used as an input to another module, for example a therapy engine module 4320.

In one form of the present technology, the output values include the interface pressure Pm, the vent flow rate Qv, the respiratory flow rate Qr, and the leak flow rate Ql.

In various forms of the present technology, the pre-processing module 4310 comprises one or more of the following algorithms: interface pressure estimation 4312, vent flow rate estimation 4314, leak flow rate estimation 4316, and respiratory flow rate estimation 4318.

### 5.4.3.1.1 Interface Pressure Estimation

In one form of the present technology, an interface pressure estimation algorithm 4312 receives as inputs a signal from the pressure sensor 4272 indicative of the pressure in the pneumatic path proximal to an outlet of the pneumatic block (the device pressure Pd) and a signal from the flow rate sensor 4274 representative of the flow rate of the airflow leaving the RPT device 4000 (the device flow rate Qd). The device flow rate Qd, absent any supplementary gas 4180, may be used as the total flow rate Qt. The interface pressure algorithm 4312 estimates the pressure drop JP through the air circuit 4170. The dependence of the pressure drop ΔP on the total flow rate Qt may be modelled for the particular air circuit 4170 by a pressure drop characteristic ΔP(Q). The interface pressure estimation algorithm, 4312 then provides as an output an estimated pressure, Pm, in the patient interface 3000. The pressure, Pm, in the patient interface 3000 may be estimated as the device pressure Pd minus the air circuit pressure drop JP.

### 5.4.3.1.2 Vent Flow Rate Estimation

In one form of the present technology, a vent flow rate estimation algorithm 4314 receives as an input an estimated pressure, Pm, in the patient interface 3000 from the interface pressure estimation algorithm 4312 and estimates a vent flow rate of air, Qv, from a vent 3400 in a patient interface 3000. The dependence of the vent flow rate Qv on the interface pressure Pm for the particular vent 3400 in use may be modelled by a vent characteristic Qv(Pm).

### 5.4.3.1.3 Leak Flow Rate Estimation

In one form of the present technology, a leak flow rate estimation algorithm 4316 receives as an input a total flow rate, Qt, and a vent flow rate Qv, and provides as an output an estimate of the leak flow rate Ql. In one form, the leak flow rate estimation algorithm estimates the leak flow rate Ql by calculating an average of the difference between total flow rate Qt and vent flow rate Qv over a period sufficiently long to include several breathing cycles, e.g. about 10 seconds.

In one form, the leak flow rate estimation algorithm 4316 receives as an input a total flow rate Qt, a vent flow rate Qv, and an estimated pressure, Pm, in the patient interface 3000, and provides as an output a leak flow rate Ql, by calculating a leak conductance, and determining a leak flow rate Ql to be a function of leak conductance and pressure, Pm. Leak conductance is calculated as the quotient of low pass filtered non-vent flow rate equal to the difference between total flow rate Qt and vent flow rate Qv, and low pass filtered square root of pressure Pm, where the low pass filter time constant has a value sufficiently long to include several breathing cycles, e.g. about 10 seconds. The leak flow rate Ql may be estimated as the product of leak conductance and a function of pressure, Pm.

### 5.4.3.1.4 Respiratory Flow Rate Estimation

In one form of the present technology, a respiratory flow rate estimation algorithm 4318 receives as an input a total flow rate, Qt, a vent flow rate, Qv, and a leak flow rate, Ql, and estimates a respiratory flow rate of air, Qr, to the patient, by subtracting the vent flow rate Qv and the leak flow rate Ql from the total flow rate Qt.

### 5.4.3.2 Therapy Engine Module

In one form of the present technology, a therapy engine module 4320 receives as inputs one or more of a pressure, Pm, in a patient interface 3000, and a respiratory flow rate of air to a patient, Qr, and provides as an output one or more therapy parameters.

In one form of the present technology, a therapy parameter is a treatment pressure Pt.

In one form of the present technology, therapy parameters are one or more of an amplitude of a pressure variation, a base pressure, and a target ventilation.

In various forms, the therapy engine module 4320 comprises one or more of the following algorithms: phase determination 4321, waveform determination 4322, ventilation determination 4323, inspiratory flow limitation determination 4324, apnea/hypopnea determination 4325, snore determination 4326, airway patency determination 4327, target ventilation determination 4328, and therapy parameter determination 4329.

### 5.4.3.2.1 Phase Determination

In one form of the present technology, the RPT device 4000 does not determine phase.

In one form of the present technology, a phase determination algorithm 4321 receives as an input a signal indicative of respiratory flow rate, Qr, and provides as an output a phase Φ of a current breathing cycle of a patient 1000.

In some forms, known as discrete phase determination, the phase output D is a discrete variable. One implementation of discrete phase determination provides a bi-valued phase output Φ with values of either inhalation or exhalation, for example represented as values of 0 and 0.5 revolutions respectively, upon detecting the start of spontaneous inhalation and exhalation respectively. RPT devices 4000 that “trigger” and “cycle” effectively perform discrete phase determination, since the trigger and cycle points are the instants at which the phase changes from exhalation to inhalation and from inhalation to exhalation, respectively. In one implementation of bi-valued phase determination, the phase output Φ is determined to have a discrete value of 0 (thereby “triggering” the RPT device 4000) when the respiratory flow rate Qr has a value that exceeds a positive threshold, and a discrete value of 0.5 revolutions (thereby “cycling” the RPT device 4000) when a respiratory flow rate Qr has a value that is more negative than a negative threshold. The inhalation time Ti and the exhalation time Te may be estimated as typical values over many respiratory cycles of the time spent with phase Φ equal to 0 (indicating inspiration) and 0.5 (indicating expiration) respectively.

Another implementation of discrete phase determination provides a tri-valued phase output Φ with a value of one of inhalation, mid-inspiratory pause, and exhalation.

In other forms, known as continuous phase determination, the phase output Φ is a continuous variable, for example varying from 0 to 1 revolutions, or 0 to 2π radians. RPT devices 4000 that perform continuous phase determination may trigger and cycle when the continuous phase reaches 0 and 0.5 revolutions, respectively. In one implementation of continuous phase determination, a continuous value of phase Φ is determined using a fuzzy logic analysis of the respiratory flow rate Qr. A continuous value of phase determined in this implementation is often referred to as “fuzzy phase”. In one implementation of a fuzzy phase determination algorithm 4321, the following rules are applied to the respiratory flow rate Qr:
1. If Qr is zero and increasing fast then Φ is 0 revolutions.
2. If Qr is large positive and steady then Φ is 0.25 revolutions.
3. If Qr is zero and falling fast, then Φ is 0.5 revolutions.
4. If Qr is large negative and steady then Φ is 0.75 revolutions.
5. If Qr is zero and steady and the 5-second low-pass filtered absolute value of Qr is large then Φ is 0.9 revolutions.
6. If Qr is positive and the phase is expiratory, then Φ is 0 revolutions.
7. If Qr is negative and the phase is inspiratory, then Φ is 0.5 revolutions.
8. If the 5-second low-pass filtered absolute value of Qr is large, Φ is increasing at a steady rate equal to the patient's breathing rate, low-pass filtered with a time constant of 20 seconds.

The output of each rule may be represented as a vector whose phase is the result of the rule and whose magnitude is the fuzzy extent to which the rule is true. The fuzzy extent to which the respiratory flow rate is “large”, “steady”, etc. is determined with suitable membership functions. The results of the rules, represented as vectors, are then combined by some function such as taking the centroid. In such a combination, the rules may be equally weighted, or differently weighted.

In another implementation of continuous phase determination, the phase Φ is first discretely estimated from the respiratory flow rate Qr as described above, as are the inhalation time Ti and the exhalation time Te. The continuous phase Φ at any instant may be determined as the half the proportion of the inhalation time Ti that has elapsed since the previous trigger instant, or 0.5 revolutions plus half the proportion of the exhalation time Te that has elapsed since the previous cycle instant (whichever instant was more recent).

### 5.4.3.2.2 Waveform Determination

In one form of the present technology, the therapy parameter determination algorithm 4329 provides an approximately constant treatment pressure throughout a respiratory cycle of a patient.

In other forms of the present technology, the therapy control module 4330 controls the pressure generator 4140 to provide a treatment pressure Pt that varies as a function of phase Φ of a respiratory cycle of a patient according to a waveform template Π(Φ).

In one form of the present technology, a waveform determination algorithm 4322 provides a waveform template Π(Φ) with values in the range [0, 1] on the domain of phase values Φ provided by the phase determination algorithm 4321 to be used by the therapy parameter determination algorithm 4329.

In one form, suitable for either discrete or continuously-valued phase, the waveform template Π(Φ) is a square-wave template, having a value of 1 for values of phase up to and including 0.5 revolutions, and a value of 0 for values of phase above 0.5 revolutions. In one form, suitable for continuously-valued phase, the waveform template Π(Φ) comprises two smoothly curved portions, namely a smoothly curved (e.g. raised cosine) rise from 0 to 1 for values of phase up to 0.5 revolutions, and a smoothly curved (e.g. exponential) decay from 1 to 0 for values of phase above 0.5 revolutions. In one form, suitable for continuously-valued phase, the waveform template Π(Φ) is based on a square wave, but with a smooth rise from 0 to 1 for values of phase up to a “rise time” that is less than 0.5 revolutions, and a smooth fall from 1 to 0 for values of phase within a “fall time” after 0.5 revolutions, with a “fall time” that is less than 0.5 revolutions.

In some forms of the present technology, the waveform determination algorithm 4322 selects a waveform template Π(Φ) from a library of waveform templates, dependent on a setting of the RPT device. Each waveform template Π(Φ) in the library may be provided as a lookup table of values Π against phase values Φ. In other forms, the waveform determination algorithm 4322 computes a waveform template Π(Φ) “on the fly” using a predetermined functional form, possibly parametrised by one or more parameters (e.g. time constant of an exponentially curved portion). The parameters of the functional form may be predetermined or dependent on a current state of the patient 1000.

In some forms of the present technology, suitable for discrete bi-valued phase of either inhalation (Φ=0 revolutions) or exhalation (Φ=0.5 revolutions), the waveform determination algorithm 4322 computes a waveform template Π“on the fly” as a function of both discrete phase Φ and time t measured since the most recent trigger instant. In one such form, the waveform determination algorithm 4322 computes the waveform template Π(Φ, t) in two portions (inspiratory and expiratory) as follows:

$$\prod ( Φ , t ) = \{ \begin{matrix}{\prod }_{i} ( t ) , & Φ = 0 \\ {\prod }_{e} ( t - {T}_{i} ) , & Φ = 0.5\end{matrix}$$

where Πᵢ(t) and Πₑ(t) are inspiratory and expiratory portions of the waveform template Π(Φ, t). In one such form, the inspiratory portion Πᵢ(t) of the waveform template is a smooth rise from 0 to 1 parametrised by a rise time, and the expiratory portion Πₑ(t) of the waveform template is a smooth fall from 1 to 0 parametrised by a fall time.

### 5.4.3.2.3 Ventilation Determination

In one form of the present technology, a ventilation determination algorithm 4323 receives an input a respiratory flow rate Qr, and determines a measure indicative of current patient ventilation, Vent.

In some implementations, the ventilation determination algorithm 4323 determines a measure of ventilation Vent that is an estimate of actual patient ventilation. One such implementation is to take half the absolute value of respiratory flow rate, Qr, optionally filtered by low-pass filter such as a second order Bessel low-pass filter with a corner frequency of 0.11 Hz.

In other implementations, the ventilation determination algorithm 4323 determines a measure of ventilation Vent that is broadly proportional to actual patient ventilation. One such implementation estimates peak respiratory flow rate Qpeak over the inspiratory portion of the cycle. This and many other procedures involving sampling the respiratory flow rate Qr produce measures which are broadly proportional to ventilation, provided the flow rate waveform shape does not vary very much (here, the shape of two breaths is taken to be similar when the flow rate waveforms of the breaths normalised in time and amplitude are similar). Some simple examples include the median positive respiratory flow rate, the median of the absolute value of respiratory flow rate, and the standard deviation of flow rate. Arbitrary linear combinations of arbitrary order statistics of the absolute value of respiratory flow rate using positive coefficients, and even some using both positive and negative coefficients, are approximately proportional to ventilation. Another example is the mean of the respiratory flow rate in the middle K proportion (by time) of the inspiratory portion, where 0<K<1. There is an arbitrarily large number of measures that are exactly proportional to ventilation if the flow rate shape is constant.

### 5.4.3.2.4 Determination of Inspiratory Flow Limitation

In one form of the present technology, the central controller 4230 executes an inspiratory flow limitation determination algorithm 4324 for the determination of the extent of inspiratory flow limitation.

In one form, the inspiratory flow limitation determination algorithm 4324 receives as an input a respiratory flow rate signal Qr and provides as an output a metric of the extent to which the inspiratory portion of the breath exhibits inspiratory flow limitation.

In one form of the present technology, the inspiratory portion of each breath is identified by a zero-crossing detector. A number of evenly spaced points (for example, sixty-five), representing points in time, are interpolated by an interpolator along the inspiratory flow rate-time curve for each breath. The curve described by the points is then scaled by a scalar to have unity length (duration/period) and unity area to remove the effects of changing breathing rate and depth. The scaled breaths are then compared in a comparator with a pre-stored template representing a normal unobstructed breath, similar to the inspiratory portion of the breath shown in FIG. 6A. Breaths deviating by more than a specified threshold (typically 1 scaled unit) at any time during the inspiration from this template, such as those due to coughs, sighs, swallows and hiccups, as determined by a test element, are rejected. For non-rejected data, a moving average of the first such scaled point is calculated by the central controller 4230 for the preceding several inspiratory events. This is repeated over the same inspiratory events for the second such point, and so on. Thus, for example, sixty-five scaled data points are generated by the central controller 4230, and represent a moving average of the preceding several inspiratory events, e.g., three events. The moving average of continuously updated values of the (e.g., sixty-five) points are hereinafter called the “scaled flow rate”, designated as Qs(t). Alternatively, a single inspiratory event can be utilised rather than a moving average.

From the scaled flow rate, two shape factors relating to the determination of partial obstruction may be calculated.

Shape factor 1 is the ratio of the mean of the middle (e.g. thirty-two) scaled flow rate points to the mean overall (e.g. sixty-five) scaled flow rate points. Where this ratio is in excess of unity, the breath will be taken to be normal. Where the ratio is unity or less, the breath will be taken to be obstructed. A ratio of about 1.17 is taken as a threshold between partially obstructed and unobstructed breathing, and equates to a degree of obstruction that would permit maintenance of adequate oxygenation in a typical patient.

Shape factor 2 is calculated as the RMS deviation from unit scaled flow rate, taken over the middle (e.g. thirty-two) points. An RMS deviation of about 0.2 units is taken to be normal. An RMS deviation of zero is taken to be a totally flow-limited breath. The closer the RMS deviation to zero, the breath will be taken to be more flow limited.

Shape factors 1 and 2 may be used as alternatives, or in combination. In other forms of the present technology, the number of sampled points, breaths and middle points may differ from those described above. Furthermore, the threshold values can be other than those described.

### 5.4.3.2.5 Determination of Apneas and Hypopneas

In one form of the present technology, the central controller 4230 executes an apnea/hypopnea determination algorithm 4325 for the determination of the presence of apneas and/or hypopneas.

In one form, the apnea/hypopnea determination algorithm 4325 receives as an input a respiratory flow rate signal Qr and provides as an output a flag that indicates that an apnea or a hypopnea has been detected.

In one form, an apnea will be said to have been detected when a function of respiratory flow rate Qr falls below a flow rate threshold for a predetermined period of time. The function may determine a peak flow rate, a relatively short-term mean flow rate, or a flow rate intermediate of relatively short-term mean and peak flow rate, for example an RMS flow rate. The flow rate threshold may be a relatively long-term measure of flow rate.

In one form, a hypopnea will be said to have been detected when a function of respiratory flow rate Qr falls below a second flow rate threshold for a predetermined period of time. The function may determine a peak flow, a relatively short-term mean flow rate, or a flow rate intermediate of relatively short-term mean and peak flow rate, for example an RMS flow rate. The second flow rate threshold may be a relatively long-term measure of flow rate. The second flow rate threshold is greater than the flow rate threshold used to detect apneas.

### 5.4.3.2.6 Determination of Snore

In one form of the present technology, the central controller 4230 executes one or more snore determination algorithms 4326 for the determination of the extent of snore.

In one form, the snore determination algorithm 4326 receives as an input a respiratory flow rate signal Qr and provides as an output a metric of the extent to which snoring is present.

The snore determination algorithm 4326 may comprise the step of determining the intensity of the flow rate signal in the range of 30-300 Hz. Further, the snore determination algorithm 4326 may comprise a step of filtering the respiratory flow rate signal Qr to reduce background noise, e.g., the sound of airflow in the system from the blower.

### 5.4.3.2.7 Determination of Airway Patency

In one form of the present technology, the central controller 4230 executes one or more airway patency determination algorithms 4327 for the determination of the extent of airway patency.

In one form, the airway patency determination algorithm 4327 receives as an input a respiratory flow rate signal Qr, and determines the power of the signal in the frequency range of about 0.75 Hz and about 3 Hz. The presence of a peak in this frequency range is taken to indicate an open airway. The absence of a peak is taken to be an indication of a closed airway.

In one form, the frequency range within which the peak is sought is the frequency of a small forced oscillation in the treatment pressure Pt. In one implementation, the forced oscillation is of frequency 2 Hz with amplitude about 1 cmH₂O.

In one form, airway patency determination algorithm 4327 receives as an input a respiratory flow rate signal Qr, and determines the presence or absence of a cardiogenic signal. The absence of a cardiogenic signal is taken to be an indication of a closed airway.

### 5.4.3.2.8 Determination of Target Ventilation

In one form of the present technology, the central controller 4230 takes as input the measure of current ventilation, Vent, and executes one or more target ventilation determination algorithms 4328 for the determination of a target value Vtgt for the measure of ventilation.

In some forms of the present technology, there is no target ventilation determination algorithm 4328, and the target value Vtgt is predetermined, for example by hard-coding during configuration of the RPT device 4000 or by manual entry through the input device 4220.

In other forms of the present technology, such as adaptive servo-ventilation (ASV), the target ventilation determination algorithm 4328 computes a target value Vtgt from a value Vtyp indicative of the typical recent ventilation of the patient.

In some forms of adaptive servo-ventilation, the target ventilation Vtgt is computed as a high proportion of, but less than, the typical recent ventilation Vtyp. The high proportion in such forms may be in the range (80%, 100%), or (85%, 95%), or (87%, 92%).

In other forms of adaptive servo-ventilation, the target ventilation Vtgt is computed as a slightly greater than unity multiple of the typical recent ventilation Vtyp.

The typical recent ventilation Vtyp is the value around which the distribution of the measure of current ventilation Vent over multiple time instants over some predetermined timescale tends to cluster, that is, a measure of the central tendency of the measure of current ventilation over recent history. In one implementation of the target ventilation determination algorithm 4328, the recent history is of the order of several minutes, but in any case, should be longer than the timescale of Cheyne-Stokes waxing and waning cycles. The target ventilation determination algorithm 4328 may use any of the variety of well-known measures of central tendency to determine the typical recent ventilation Vtyp from the measure of current ventilation, Vent. One such measure is the output of a low-pass filter on the measure of current ventilation Vent, with time constant equal to one hundred seconds.

### 5.4.3.2.9 Determination of Therapy Parameters

In some forms of the present technology, the central controller 4230 executes one or more therapy parameter determination algorithms 4329 for the determination of one or more therapy parameters using the values returned by one or more of the other algorithms in the therapy engine module 4320.

In one form of the present technology, the therapy parameter is an instantaneous treatment pressure Pt. In one implementation of this form, the therapy parameter determination algorithm 4329 determines the treatment pressure Pt using the equation

$$Pt = A \prod ( Φ , t ) + {P}_{0} \qquad ( 1 )$$

where:
A is the amplitude,
Π(Φ, t) is the waveform template value (in the range 0 to 1) at the current value Φ of phase and t of time, and
P₀ is a base pressure.

If the waveform determination algorithm 4322 provides the waveform template Π(Φ, t) as a lookup table of values Π indexed by phase Φ, the therapy parameter determination algorithm 4329 applies equation (1) by locating the nearest lookup table entry to the current value Φ of phase returned by the phase determination algorithm 4321, or by interpolation between the two entries straddling the current value Φ of phase.

The values of the amplitude A and the base pressure P₀ may be set by the therapy parameter determination algorithm 4329 depending on the chosen respiratory pressure therapy mode in the manner described below. 5.4.3.3 Therapy Control module

The therapy control module 4330 in accordance with one aspect of the present technology receives as inputs the therapy parameters from the therapy parameter determination algorithm 4329 of the therapy engine module 4320, and controls the pressure generator 4140 to deliver a flow of air in accordance with the therapy parameters.

In one form of the present technology, the therapy parameter is a treatment pressure Pt, and the therapy control module 4330 controls the pressure generator 4140 to deliver a flow of air whose interface pressure Pm at the patient interface 3000 is equal to the treatment pressure Pt.

### 5.4.3.4 Detection of Fault Conditions

In one form of the present technology, the central controller 4230 executes one or more methods 4340 for the detection of fault conditions. The fault conditions detected by the one or more methods 4340 may include at least one of the following:
Power failure (no power, or insufficient power)
Transducer fault detection
Failure to detect the presence of a component
Operating parameters outside recommended ranges (e.g. pressure, flow rate, temperature, PaO2)
Failure of a test alarm to generate a detectable alarm signal.

Upon detection of the fault condition, the corresponding algorithm 4340 signals the presence of the fault by one or more of the following:
Initiation of an audible, visual &/or kinetic (e.g. vibrating) alarm
Sending a message to an external device
Logging of the incident

### 5.4.4 Self-Contained Flow Generator

As illustrated in FIGS. 27 to 31, an RPT device 6500 may be disposed within the flow generator casing 6400 of the patient interface 6000 (or any of the patient interfaces). For example, the RPT device 6500 may be disposed or enclosed within the cavity 6408 formed between the front case 6404 and the rear case 6406. The front case 6404 and the rear case 6406 may be formed from a rigid or semi-rigid material in order to protect the components housed in the cavity 6408.

In some forms, the RPT device 6500 may include a blower 6502. The blower 6502 may be substantially cylindrical in shape and arranged laterally within the cavity 6408.

In some forms, the RPT device 6500 may include a suspension 6504. The suspension 6504 may receive and support the blower 6502 within the cavity 6408.

As illustrated in FIG. 31, the suspension 6504 may include at least one opening, e.g. two openings corresponding to the air intakes at the ends of the motor 6502, e.g., as seen in FIG. 48. As shown in FIG. 48, motor 6502 may have at least a pair of impellers 6550 connected to a common shaft and configured to pressurize breathable air in a range suitable for therapy. The impellers are arranged in series and their combined flows are released at one or more central outlets near the center of the motor 6502.

Turning back to FIG. 31, the suspension 6504 may be formed as a substantially cylindrical body (e.g., similar to the blower 6502) and may include at least a first opening 6506 along a longitudinal direction of the suspension 6504. Only one opening is visible in FIG. 31, but the other end of the suspension 6504 may include a second opening (for the opposed impeller on that side). The first opening 6506 may be sized in order to receive the blower 6502, and each first opening is sized to allow intake air from ambient to flow into the respective impeller.

The suspension 6504 may also include a second opening 6508 that extends tangentially from the cylindrical surface of the suspension 6504. The second opening 6508 may be in fluid communication with the first opening 6506. In use, airflow generated by the blower 6502 and output from the at least one central outlet of the blower 6502 may be forced through the second opening 6504 and toward the plenum chamber 6200.

In some forms, a manifold 6510 may be connected to the suspension 6504 proximate to the second opening 6508. For example, FIGS. 28 and 29 illustrate the manifold 6510 directly connected to the suspension 6504 at the second opening 6508, and extends away from the cylindrical body of the suspension 6504.

As shown in FIG. 29, the manifold 6510 may be hollow and may form an airflow path with the suspension 6504 in order to assist in conveying pressurized air from the blower 6502 to the plenum chamber 6200. The manifold 6510 may include a first opening 6512 directly connected to the second opening 6508 of the suspension 6504. The manifold 6510 may also include a second opening 6514 at an opposite end from the first opening 6512. Airflow may therefore flow through the manifold 6510, entering through the first opening 6512 and exiting through the second opening 6514.

In some forms, the first opening 6512 and the second opening 6514 may be oriented substantially perpendicular with respect to one another. The manifold 6510 may therefore be able to change the direction of the airflow path. For example, the airflow path may travel in the superior direction of FIG. 29 through the first opening 6512 before traveling in a posterior direction (e.g., to the right as illustrated in FIG. 29) through the second opening 6514.

In some forms, an expiratory activated valve (EAV) 6516 may be connected to the manifold 6510. For example, the EAV 6516 may be connected adjacent to the second opening 6514 of the manifold 6510. The EAV 6516 may be described in more detail below. However, the EAV 6516 may include at least one airflow path so that the pressurized air exiting the second opening 6514 of the manifold 6510 may continue to be directed toward the plenum chamber 6200.

As illustrated in FIG. 29, the EAV 6516 may be positioned at least partially within an inlet 6518, e.g., in the form of an inlet tube, of the rear case 6468. The plenum chamber 6200 may be connected to the rear case 6406, and the inlet 6518 may extend into the plenum chamber 6200 so that airflow through the inlet 6518 enters the plenum chamber 6200. Thus, there may be an airflow path for delivering the pressurized air from the blower 6502 to the plenum chamber 6200. As illustrated in FIG. 31, the rear case 6406 may include a central groove or recess 6410, which is part of the cavity 6408 when the front case 6404 is connected. The central groove or recess 6410 may be semi-cylindrical in shape, and may be configured to receive the blower 6502 and the suspension 6504. The plenum chamber 6200 includes an anterior surface with a concave section that receives a convex exterior part of the casing or rear case that houses the blower, as shown in FIGS. 29 and 31. The casing, e.g., rear case 6408, includes an inlet tube, e.g., somewhat oval in shape to match the shape of the valve shown in FIGS. 44-47) positioned above the convex exterior part of the casing or rear case, and the plenum chamber includes an inlet opening that may be provided in a more rigid part of the seal-forming portion.

In some forms, the central groove or recess 6410 may be substantially linear, but the rear case 6406 may curve as described above. One or more mufflers 6412 may be connected to the rear case 6406 within the cavity 6408 and outside of the central groove 6410. In other words, a muffler 6412 may be positioned at one or both ends of the blower 6502. The mufflers 6512, therefore, may assist in reducing the noise output of the blower 6502. This may be useful because the blower 6502 is positioned proximate to the patient's face and could cause sleep disturbances because of the noise.

Returning to FIGS. 27 and 29, the casing, e.g., rear case and/or front case 6404, may include outlet vents 6414. Although in other examples, the outlet vents 6414 may be included on the rear case 6406. The outlet vents 6414 may allow exhaled air to exhaust to ambient.

In the illustrated example (see e.g., FIG. 29), the rear case 6406 may include an exhaust channel 6416. The EAV 6516 may be smaller than the inlet 6518 so that airflow may pass around the outside of the EAV 6516. The exhausted air flowing around the outside of the EAV 6516 (e.g., air exiting the plenum chamber 6200) may be directed by the EAV 6516 into the exhaust channel 6416 and through the outlet vents 6414.

### 5.4.4.1 Blower

As shown in FIG. 48, the interior of the blower 6502 may be shown. The blower 6502 may include at least one impeller 6550 (see e.g., FIG. 49), e.g., at least one pair of opposed impellers arranged in parallel within the blower housing 6552 and mounted on the common shaft. For example, the illustrated blower 6502 may include at least a pair or two pair (four impellers) 6550, e.g., a first pair of impellers, on each side of the magnet 6554 and the winding 6556 and/or a second pair of impellers, again with one on each side of the magnet.

As shown in FIGS. 50 and 51, an alternate version of the impeller 6550-1 is shown. The impeller 6550-1 may be a double sided impeller and may achieve the same pressure as the impeller 6550 (e.g., in FIG. 49) in a smaller space. This may be beneficial in order to maximize the limited space within the flow generator casing 6400.

In some forms, an elastomer bearing 6558 may be coupled between a common shaft on which the impellers are mounted and the impeller 6550-1. The elastomer bearing 6558 may limit vibrations in the blower 6502.

In some forms, the blower may be similar to the blower described in U.S. patent application Ser. Nos. 16/320,565 and 17/602,552, both of which are incorporated herein by reference in their entirety.

### 5.5 Air Circuit

An air circuit 4170 in accordance with an aspect of the present technology is a conduit or a tube constructed and arranged to allow, in use, a flow of air to travel between two components such as RPT device 4000 and the patient interface 3000.

In particular, the air circuit 4170 may be in fluid connection with the outlet of the pneumatic block 4020 and the patient interface. The air circuit may be referred to as an air delivery tube. In some cases there may be separate limbs of the circuit for inhalation and exhalation. In other cases a single limb is used.

In some forms, the air circuit 4170 may comprise one or more heating elements configured to heat air in the air circuit, for example to maintain or raise the temperature of the air. The heating element may be in a form of a heated wire circuit, and may comprise one or more transducers, such as temperature sensors. In one form, the heated wire circuit may be helically wound around the axis of the air circuit 4170. The heating element may be in communication with a controller such as a central controller 4230. One example of an air circuit 4170 comprising a heated wire circuit is described in U.S. Pat. No. 8,733,349, which is incorporated herewithin in its entirety by reference.

### 5.5.1 Valve Assembly

As described above, the patient interface 6000 (or any of the described patient interfaces) may include an EAV 6516. The EAV 6516 may control airflow through the patient interface. Although an EAV 6516 is described below, other types of valves may replace the EAV 6516 without departing for the scope of the examples.

As illustrated in FIG. 32, the EAV 6516 may be shaped like a disc. The EAV 6516 may include a retention flange 6520. The retention flange 6520 may form an outer perimeter of the EAV 6516 and may assist in locating and/or retaining the EAV 6516 in a desired position.

In some forms, a center of the EAV 6516 may include a duckbill valve 6522, which may allow air to pass through the EAV 6516 in one direction only. The duckbill valve 6522 may block airflow through the EAV 6516 in the opposite direction.

In some forms, the EAV 6516 includes a membrane 6524 connecting the retention flange 6520 to the duckbill valve 6522. The retention flange 6520 may be the only part of the EAV 6516 to be fixed on assembly allowing the membrane 6524 to move relative to the pressure differential acting on either side of the EAV 6516. Although they may be formed as one part, the duckbill valve 6522 and membrane 6524 may work independently to each other. The duckbill valve 6522 may control airflow from the RPT device (e.g., blower 6502) to the patient interface (e.g., plenum chamber 6200), and the membrane 6524 controls the airflow from the patient interface (e.g., the plenum chamber 6524) to the outlet vent 6414. The EAV 6516 may be a dynamic component, reacting to every breath that the patient takes.

In some forms, the EAV 6516 is constructed from a flexible material (e.g., silicone). The EAV 6516 may be formed with LSR and/or CMSR. The EAV 6516 may also have a shore hardness of 30-60 Shore (A).

As shown in FIGS. 33 and 34, the EAV 6516 may have duckbill valve 6522 in the center of the membrane 6524. The duckbill valve 6522 may be a one-way valve allowing air to pass through the membrane 6524 in one direction only. The pressure differential acting on either side of the duckbill valve 6522 will determine if the EAV 6516 is open or closed. The size and shape of the duckbill valve 6522 will have an effect on the flow rate and impedance through the membrane

For example, as illustrated in FIG. 33, a high pressure is located proximate to the duckbill valve 6522 and a low pressure is located proximate to the flange 6520. The illustrated pressure differential draws air toward the duckbill valve 6522 in order to reach the low pressure. However, the airflow contacts the sides (e.g., an outer surface) of the duckbill valve 6522, and maintains the duckbill valve in the closed position.

Alternatively, as illustrated in FIG. 34, a low pressure is located proximate to the duckbill valve 6522 and a high pressure is located proximate to the flange 6520. The illustrated pressure differential draws air toward the inner surface of the duckbill valve 6522 in order to reach the low pressure outside of the duckbill valve 6522. Unlike in FIG. 33, flow in the direction illustrate in FIG. 34 opens the duckbill valve 6522 so that the higher pressure air may reach the lower pressure air.

As illustrated in FIG. 35, the EAV 6516 may have a thin membrane 6524 connecting the outer diameter (e.g., proximate to the flange 6520) to the duckbill valve 6522 in the center. The membrane 6524 may connect to the duckbill valve 6522 at a connection section 6526 (e.g., a small flat section).

In one form, the membrane 6524 has a trampoline-like ability moving up or down relative to the pressure differential acting on either side of the membrane 6524. The movement of the membrane 6524 does not affect the shape or performance of the outer diameter (e.g., proximate to the ridge 6520) and/or duckbill valve 6522.

In some forms, the retention flange 6520 may locate the EAV 6516 against the manifold 6510. The retention flange 6520 may also be a seal between the RPT device (e.g., blower 6502) and mask air path (e.g., from the manifold 6510 to the inlet 6518), and between the RPT device and outlet vent 6414. Unintentional leak (e.g., airflow generated by the blower 6502 that does not reach the plenum chamber 6200) may be considered wasted effort and may affect the workings of the EAV 6516. When integrated, the retention flange 6520 may be the only part of the EAV 6516 to be fixed on assembly therefore allowing the membrane 6524 to move relative to the pressure differential acting on either side.

In one form, the present technology comprises a pressure sensor connected to the patient interface. A controller (for example the therapy device controller 4240) monitors the pressure in the patient interface measured by the pressure sensor and controls the pressure generator 4140 to adjust the flow rate of the generated air supply.

The retention flange 6520 of the EAV 6516 may sit within a recess of one part of the flow generator casing 6400 (e.g., the inlet 6518 of the rear case 6406). This feature may ensure the EAV 6516 sits concentric to the components of the flow generator casing 6400 and limits any lateral movement.

When assembled, the retention flange 6520 of the EAV 6516 creates an interference seal with the manifold 6510.

The section 6526 of the valve member membrane 6524 that interfaces with the inlet 6518 may align vertically with a rib 6418 extending inwardly from the inlet 6518. The clearance gap between the valve member membrane 6524 and rib 6418 may control the ambient port flow. The holes of the outlet vent 6414 are larger and more open to atmosphere than the clearance gap. They may be considered a secondary ambient port and have less effect on the ambient port flow itself than the size of the clearance gap but may provide more of a protection shroud for the valve member membrane 6524.

As shown in FIG. 36, on inspiration the patient interface pressure drops due to the increase of volume created by the user's diaphragm contracting (moving down). The pressure sensor records this change and signals the RPT device 6500 to provide more airflow to maintain the set CPAP pressure. The RPT device airflow will cause an increase in pressure on the RPT device side of the EAV 6516. This pressure is greater than the patient interface pressure (e.g., the pressure in the plenum chamber 6200) and causes the valve member membrane 6524 to move until contact is made with the patient interface port rib 6418, reducing airflow loss. Simultaneously the silicone duckbill valve 6522 will open allowing air flow into the plenum chamber 6200. This airflow will cause the patient interface pressure to increase until the desired CPAP pressure is reached.

In certain forms as shown in FIG. 37, on inspiration the RPT device 6500 is signalled to provide an air flow to pressurise the plenum chamber 6200 and user's airway. The air flow from the blower 6502 will cause a build-up of pressure on the RPT device side of the EAV 6516. This pressure build-up results in the valve member membrane 6524 moving until contact is made with the patient interface port rib 6418. The pressure differential to atmosphere causes the valve member membrane 6524 to stay in this position during inspiration reducing air flow loses and thus efficiencies.

In certain forms as shown in FIG. 38, on inspiration the pressure proximate to the blower 6502 is greater than the pressure within the plenum chamber 6200. The silicone duckbill valve 6522 will open allowing air flow into the plenum chamber 6200. This air flow will cause the pressure in the plenum chamber 6200 to increase until the desired CPAP pressure is reached.

As shown in FIG. 39, on expiration the pressure in the plenum chamber 6200 increases due to the decrease of volume created by the user's diaphragm relaxing (moving up). The pressure sensor records this change and signals the blower 6502 to stop or slow down. The pressure in the plenum chamber 6200 is therefore greater than the pressure on the side of the EAV 6516 proximate to the blower 6502, and the valve member membrane 6524 moves away from the patient interface port rib 6418, introducing an airflow path to atmosphere (e.g., through the outlet vent 6414). Simultaneously the duckbill valve 6522 will close ensuring no reverse airflow travels back toward the blower 6502 (e.g., through the manifold 6510). The pressure inside the plenum chamber 6200 will decrease as airflow is released to atmosphere (e.g., through the outlet vent 6414). The pressure sensor will monitor this pressure drop and the airflow from the blower 6502 will remain off (or reduced) until the patient interface pressure falls below the set CPAP pressure.

In certain forms as shown in FIG. 40, on expiration the pressure in the plenum chamber 6200 is greater than RPT device pressure (e.g., the pressure on the side of the EAV 6516 proximate to the blower 6502). This pressure differential allows the valve member membrane 6524 to move away from the patient interface port rib 6418, introducing an airflow path to atmosphere (E.g., through the outlet vent 6414). This clearance gap 6420 is considered the primary ambient port on the EAV 6516 and has the biggest influence on the flow rate through the outlet vent 6414. The vented airflow then moves into a chamber 6422 and out of secondary ambient ports 6424, which may be formed in the rear case 6406 radially outside of the inlet 6518. The ambient ports (e.g., the clearance gap 6420, the secondary ambient port 6424, and the outlet vent 6414) provide protection for the EAV 6516, protecting it from foreign bodies (e.g., backflow from debris).

In certain forms as shown in FIG. 41, when the pressure in the plenum chamber 6200 is greater than the pressure in the suspension 6504, the silicone duckbill valve 6522 will close. Therefore, no user airflow on expiration will flow back into the manifold 6510 toward the blower 6502.

In certain forms, the EAV 6516 will dynamically move between the inspiration position (see e.g., FIGS. 36 to 38) and expiration position (see e.g., FIGS. 39 to 41) during the patient's breathing cycles. The valve assembly may be called an expiratory activated valve (EAV), but it is constantly reacting to all stages of the breathing cycle and the simplified, exemplary illustrations within this document only describe the extremes.

As highlighted above, the EAV 6500 only vents to atmosphere on expiration in certain forms of the technology. Therefore, the vent flow curve may not follow the constant flow characteristic of a traditional system, but may more follow an ‘on-off’ curve shape. All the vent flow that may occur is powered by the patient's effort, thus reducing vent flow losses on inspiration and improving electrical and blower efficiencies when compared to a tradition CPAP.

On a traditional patient interface, when a patient expires, the increase in air volume will cause an increase in pressure in the plenum chamber (e.g., pressure swing). The additional pressure will dissipate as flow out the vent holes but also down the air circuit. Therefore, a traditional patient interface system has a larger internal volume than that of the present technology. In accordance with some forms of the present technology, all the expired airflow may now exit the valve assembly. To reduce the risk of pressure increases (swings) the ambient port of the valve assembly may allow a higher flow rate, reducing the impedance to atmosphere.

As illustrated in FIGS. 42 to 47, the EAV 6516 may be designed with different shapes in order to different impedances and/or deflections. Although the different EAVs 6516 may be shaped differently, each may provide the same functionality described above.

As shown in FIGS. 42 and 43, a first version of the EAV 6516-1 may include a flange 6520 with a generally circular perimeter. The duckbill valve 6522 may be positioned within the circumference of the flange 6520 (as described above), and may also include a generally circular shape.

As shown in FIGS. 44 and 45, a second version of the EAV 6516-2 may include a flange 6520 with a generally elliptical shape. The duckbill valve 6522 may similarly have an elliptical shape.

As shown in FIGS. 46 and 47, a third version of the EAV 6516-3 may include a flange 6520 with a generally elliptical shape. The duckbill valve 6522 may similarly have an elliptical shape. The elliptical shape of the third version 6516-3 may be more rounded than the second version 6516-2 (e.g., less elongated), but may not be fully circular like the first version 6516-1.

In some forms, the difference versions may be used to achieve different impedances. For example, an EAV with a more rounded shape (e.g., approximating a circle) may include a higher impedance. The first version 6516-1 may therefore include the highest impedance and the second version 6516-2 may include the lowest impedance.

In some forms, the different versions may also include different levels of deflection. For example, the more rounded versions (e.g., the first and third versions 6516-1, 6516-3) may include a higher displacement of the membrane 6524 at a given pressure for both exhalation and inhalation.

As shown in FIG. 52, a controller may determine the pressure at the blower 6502 (e.g., before the EAV 6516), the pressure in the plenum chamber 6200 (e.g., after the EAV 6516), and the flow of the blower 6502. The controller may use these data points to create a vent flow estimation model (e.g., using the calculation of FIG. 53), which can be used to estimate the vent flow. In some forms, the control may control the blower 6502 in order to optimize the vent flow.

### 5.6 Humidifier

### 5.6.1 Humidifier Overview

In one form of the present technology there is provided a humidifier 5000 (e.g. as shown in FIG. 5A) to change the absolute humidity of air or gas for delivery to a patient relative to ambient air. Typically, the humidifier 5000 is used to increase the absolute humidity and increase the temperature of the flow of air (relative to ambient air) before delivery to the patient's airways.

The humidifier 5000 may comprise a humidifier reservoir 5110, a humidifier inlet 5002 to receive a flow of air, and a humidifier outlet 5004 to deliver a humidified flow of air. In some forms, as shown in FIG. 5A and FIG. 5B, an inlet and an outlet of the humidifier reservoir 5110 may be the humidifier inlet 5002 and the humidifier outlet 5004 respectively. The humidifier 5000 may further comprise a humidifier base 5006, which may be adapted to receive the humidifier reservoir 5110 and comprise a heating element 5240.

### 5.6.2 Humidifier Components

### 5.6.2.1 Water Reservoir

According to one arrangement, the humidifier 5000 may comprise a water reservoir 5110 configured to hold, or retain, a volume of liquid (e.g. water) to be evaporated for humidification of the flow of air. The water reservoir 5110 may be configured to hold a predetermined maximum volume of water in order to provide adequate humidification for at least the duration of a respiratory therapy session, such as one evening of sleep. Typically, the reservoir 5110 is configured to hold several hundred millilitres of water, e.g. 300 millilitres (ml), 325 ml, 350 ml or 400 ml. In other forms, the humidifier 5000 may be configured to receive a supply of water from an external water source such as a building's water supply system.

According to one aspect, the water reservoir 5110 is configured to add humidity to a flow of air from the RPT device 4000 as the flow of air travels therethrough. In one form, the water reservoir 5110 may be configured to encourage the flow of air to travel in a tortuous path through the reservoir 5110 while in contact with the volume of water therein.

According to one form, the reservoir 5110 may be removable from the humidifier 5000, for example in a lateral direction as shown in FIG. 5A and FIG. 5B.

The reservoir 5110 may also be configured to discourage egress of liquid therefrom, such as when the reservoir 5110 is displaced and/or rotated from its normal, working orientation, such as through any apertures and/or in between its sub-components. As the flow of air to be humidified by the humidifier 5000 is typically pressurised, the reservoir 5110 may also be configured to prevent losses in pneumatic pressure through leak and/or flow impedance.

### 5.6.2.2 Conductive Portion

According to one arrangement, the reservoir 5110 comprises a conductive portion 5120 configured to allow efficient transfer of heat from the heating element 5240 to the volume of liquid in the reservoir 5110. In one form, the conductive portion 5120 may be arranged as a plate, although other shapes may also be suitable. All or a part of the conductive portion 5120 may be made of a thermally conductive material such as aluminium (e.g. approximately 2 mm thick, such as 1 mm, 1.5 mm, 2.5 mm or 3 mm), another heat conducting metal or some plastics. In some cases, suitable heat conductivity may be achieved with less conductive materials of suitable geometry.

### 5.6.2.3 Humidifier Reservoir Dock

In one form, the humidifier 5000 may comprise a humidifier reservoir dock 5130 (as shown in FIG. 5B) configured to receive the humidifier reservoir 5110. In some arrangements, the humidifier reservoir dock 5130 may comprise a locking feature such as a locking lever 5135 configured to retain the reservoir 5110 in the humidifier reservoir dock 5130.

### 5.6.2.4 Water Level Indicator

The humidifier reservoir 5110 may comprise a water level indicator 5150 as shown in FIG. 5A-5B. In some forms, the water level indicator 5150 may provide one or more indications to a user such as the patient 1000 or a care giver regarding a quantity of the volume of water in the humidifier reservoir 5110. The one or more indications provided by the water level indicator 5150 may include an indication of a maximum, predetermined volume of water, any portions thereof, such as 25%, 50% or 75% or volumes such as 200 ml, 300 ml or 400 ml.

### 5.6.2.5 Heating Element

A heating element 5240 may be provided to the humidifier 5000 in some cases to provide a heat input to one or more of the volume of water in the humidifier reservoir 5110 and/or to the flow of air. The heating element 5240 may comprise a heat generating component such as an electrically resistive heating track. One suitable example of a heating element 5240 is a layered heating element such as one described in the PCT Patent Application Publication No. WO 2012/171072, which is incorporated herewith by reference in its entirety.

In some forms, the heating element 5240 may be provided in the humidifier base 5006 where heat may be provided to the humidifier reservoir 5110 primarily by conduction as shown in FIG. 5B.

### 5.7 Breathing Waveforms

FIG. 6A shows a model typical breath waveform of a person while sleeping. The horizontal axis is time, and the vertical axis is respiratory flow rate. While the parameter values may vary, a typical breath may have the following approximate values: tidal volume Vt 0.5 L, inhalation time Ti 1.6 s, peak inspiratory flow rate Qpeak 0.4 L/s, exhalation time Te 2.4 s, peak expiratory flow rate Qpeak −0.5 L/s. The total duration of the breath, Ttot, is about 4 s. The person typically breathes at a rate of about 15 breaths per minute (BPM), with Ventilation Vent about 7.5 L/min. A typical duty cycle, the ratio of Ti to Ttot, is about 40%.

### 5.8 Respiratory Therapy Modes

Various respiratory therapy modes may be implemented by the disclosed respiratory therapy system.

### 5.8.1 CPAP Therapy

In some implementations of respiratory pressure therapy, the central controller 4230 sets the treatment pressure Pt according to the treatment pressure equation (1) as part of the therapy parameter determination algorithm 4329. In one such implementation, the amplitude A is identically zero, so the treatment pressure Pt (which represents a target value to be achieved by the interface pressure Pm at the current instant of time) is identically equal to the base pressure P₀ throughout the respiratory cycle. Such implementations are generally grouped under the heading of CPAP therapy. In such implementations, there is no need for the therapy engine module 4320 to determine phase Φ or the waveform template Π(Φ).

In CPAP therapy, the base pressure P₀ may be a constant value that is hard-coded or manually entered to the RPT device 4000. Alternatively, the central controller 4230 may repeatedly compute the base pressure P₀ as a function of indices or measures of sleep disordered breathing returned by the respective algorithms in the therapy engine module 4320, such as one or more of flow limitation, apnea, hypopnea, patency, and snore. This alternative is sometimes referred to as APAP therapy.

FIG. 4E is a flow chart illustrating a method 4500 carried out by the central controller 4230 to continuously compute the base pressure P₀ as part of an APAP therapy implementation of the therapy parameter determination algorithm 4329, when the pressure support A is identically zero.

The method 4500 starts at step 4520, at which the central controller 4230 compares the measure of the presence of apnea/hypopnea with a first threshold, and determines whether the measure of the presence of apnea/hypopnea has exceeded the first threshold for a predetermined period of time, indicating an apnea/hypopnea is occurring. If so, the method 4500 proceeds to step 4540; otherwise, the method 4500 proceeds to step 4530. At step 4540, the central controller 4230 compares the measure of airway patency with a second threshold. If the measure of airway patency exceeds the second threshold, indicating the airway is patent, the detected apnea/hypopnea is deemed central, and the method 4500 proceeds to step 4560; otherwise, the apnea/hypopnea is deemed obstructive, and the method 4500 proceeds to step 4550.

At step 4530, the central controller 4230 compares the measure of flow limitation with a third threshold. If the measure of flow limitation exceeds the third threshold, indicating inspiratory flow is limited, the method 4500 proceeds to step 4550; otherwise, the method 4500 proceeds to step 4560.

At step 4550, the central controller 4230 increases the base pressure P₀ by a predetermined pressure increment ΔP, provided the resulting treatment pressure Pt would not exceed a maximum treatment pressure Pmax. In one implementation, the predetermined pressure increment JP and maximum treatment pressure Pmax are 1 cmH2O and 25 cmH2O respectively. In other implementations, the pressure increment ΔP can be as low as 0.1 cmH2O and as high as 3 cmH2O, or as low as 0.5 cmH2O and as high as 2 cmH2O. In other implementations, the maximum treatment pressure Pmax can be as low as 15 cmH2O and as high as 35 cmH2O, or as low as 20 cmH2O and as high as 30 cmH2O. The method 4500 then returns to step 4520.

At step 4560, the central controller 4230 decreases the base pressure P₀ by a decrement, provided the decreased base pressure P₀ would not fall below a minimum treatment pressure Pmin. The method 4500 then returns to step 4520. In one implementation, the decrement is proportional to the value of P₀−Pmin, so that the decrease in P₀ to the minimum treatment pressure Pmin in the absence of any detected events is exponential. In one implementation, the constant of proportionality is set such that the time constant τ of the exponential decrease of P₀ is 60 minutes, and the minimum treatment pressure Pmin is 4 cmH2O. In other implementations, the time constant τ could be as low as 1 minute and as high as 300 minutes, or as low as 5 minutes and as high as 180 minutes. In other implementations, the minimum treatment pressure Pmin can be as low as 0 cmH2O and as high as 8 cmH2O, or as low as 2 cmH2O and as high as 6 cmH2O. Alternatively, the decrement in P₀ could be predetermined, so the decrease in P₀ to the minimum treatment pressure Pmin in the absence of any detected events is linear.

### 5.8.2 Bi-Level Therapy

In other implementations of this form of the present technology, the value of amplitude A in equation (1) may be positive. Such implementations are known as bi-level therapy, because in determining the treatment pressure Pt using equation (1) with positive amplitude A, the therapy parameter determination algorithm 4329 oscillates the treatment pressure Pt between two values or levels in synchrony with the spontaneous respiratory effort of the patient 1000. That is, based on the typical waveform templates Π(Φ, t) described above, the therapy parameter determination algorithm 4329 increases the treatment pressure Pt to P₀+A (known as the IPAP) at the start of, or during, or inspiration and decreases the treatment pressure Pt to the base pressure P₀ (known as the EPAP) at the start of, or during, expiration.

In some forms of bi-level therapy, the IPAP is a treatment pressure that has the same purpose as the treatment pressure in CPAP therapy modes, and the EPAP is the IPAP minus the amplitude A, which has a “small” value (a few cmH2O) sometimes referred to as the Expiratory Pressure Relief (EPR). Such forms are sometimes referred to as CPAP therapy with EPR, which is generally thought to be more comfortable than straight CPAP therapy. In CPAP therapy with EPR, either or both of the IPAP and the EPAP may be constant values that are hard-coded or manually entered to the RPT device 4000. Alternatively, the therapy parameter determination algorithm 4329 may repeatedly compute the IPAP and/or the EPAP during CPAP with EPR. In this alternative, the therapy parameter determination algorithm 4329 repeatedly computes the EPAP and/or the IPAP as a function of indices or measures of sleep disordered breathing returned by the respective algorithms in the therapy engine module 4320 in analogous fashion to the computation of the base pressure P₀ in ΔPAP therapy described above.

In other forms of bi-level therapy, the amplitude A is large enough that the RPT device 4000 does some or all of the work of breathing of the patient 1000. In such forms, known as pressure support ventilation therapy, the amplitude A is referred to as the pressure support, or swing. In pressure support ventilation therapy, the IPAP is the base pressure P₀ plus the pressure support A, and the EPAP is the base pressure P₀.

In some forms of pressure support ventilation therapy, known as fixed pressure support ventilation therapy, the pressure support A is fixed at a predetermined value, e.g. 10 cmH2O. The predetermined pressure support value is a setting of the RPT device 4000, and may be set for example by hard-coding during configuration of the RPT device 4000 or by manual entry through the input device 4220.

In other forms of pressure support ventilation therapy, broadly known as servo-ventilation, the therapy parameter determination algorithm 4329 takes as input some currently measured or estimated parameter of the respiratory cycle (e.g. the current measure Vent of ventilation) and a target value of that respiratory parameter (e.g. a target value Vtgt of ventilation) and repeatedly adjusts the parameters of equation (1) to bring the current measure of the respiratory parameter towards the target value. In a form of servo-ventilation known as adaptive servo-ventilation (ASV), which has been used to treat CSR, the respiratory parameter is ventilation, and the target ventilation value Vtgt is computed by the target ventilation determination algorithm 4328 from the typical recent ventilation Vtyp, as described above.

In some forms of servo-ventilation, the therapy parameter determination algorithm 4329 applies a control methodology to repeatedly compute the pressure support A so as to bring the current measure of the respiratory parameter towards the target value. One such control methodology is Proportional-Integral (PI) control. In one implementation of PI control, suitable for ASV modes in which a target ventilation Vtgt is set to slightly less than the typical recent ventilation Vtyp, the pressure support A is repeatedly computed as:

$$A = G \int ( Vent - Vtgt ) dt \qquad ( 2 )$$

where G is the gain of the PI control. Larger values of gain G can result in positive feedback in the therapy engine module 4320. Smaller values of gain G may permit some residual untreated CSR or central sleep apnea. In some implementations, the gain G is fixed at a predetermined value, such as −0.4 cmH2O/(L/min)/sec. Alternatively, the gain G may be varied between therapy sessions, starting small and increasing from session to session until a value that substantially eliminates CSR is reached. Conventional means for retrospectively analysing the parameters of a therapy session to assess the severity of CSR during the therapy session may be employed in such implementations. In yet other implementations, the gain G may vary depending on the difference between the current measure Vent of ventilation and the target ventilation Vtgt.

Other servo-ventilation control methodologies that may be applied by the therapy parameter determination algorithm 4329 include proportional (P), proportional-differential (PD), and proportional-integral-differential (PID).

The value of the pressure support A computed via equation (2) may be clipped to a range defined as [Amin, Amax]. In this implementation, the pressure support A sits by default at the minimum pressure support Amin until the measure of current ventilation Vent falls below the target ventilation Vtgt, at which point A starts increasing, only falling back to Amin when Vent exceeds Vtgt once again.

The pressure support limits Amin and Amax are settings of the RPT device 4000, set for example by hard-coding during configuration of the RPT device 4000 or by manual entry through the input device 4220.

In pressure support ventilation therapy modes, the EPAP is the base pressure P₀. As with the base pressure P₀ in CPAP therapy, the EPAP may be a constant value that is prescribed or determined during titration. Such a constant EPAP may be set for example by hard-coding during configuration of the RPT device 4000 or by manual entry through the input device 4220. This alternative is sometimes referred to as fixed-EPAP pressure support ventilation therapy. Titration of the EPAP for a given patient may be performed by a clinician during a titration session with the aid of PSG, with the aim of preventing obstructive apneas, thereby maintaining an open airway for the pressure support ventilation therapy, in similar fashion to titration of the base pressure P₀ in constant CPAP therapy.

Alternatively, the therapy parameter determination algorithm 4329 may repeatedly compute the base pressure P₀ during pressure support ventilation therapy. In such implementations, the therapy parameter determination algorithm 4329 repeatedly computes the EPAP as a function of indices or measures of sleep disordered breathing returned by the respective algorithms in the therapy engine module 4320, such as one or more of flow limitation, apnea, hypopnea, patency, and snore. Because the continuous computation of the EPAP resembles the manual adjustment of the EPAP by a clinician during titration of the EPAP, this process is also sometimes referred to as auto-titration of the EPAP, and the therapy mode is known as auto-titrating EPAP pressure support ventilation therapy, or auto-EPAP pressure support ventilation therapy.

### 5.8.3 High Flow Therapy

In other forms of respiratory therapy, the pressure of the flow of air is not controlled as it is for respiratory pressure therapy. Rather, the central controller 4230 controls the pressure generator 4140 to deliver a flow of air whose device flow rate Qd is controlled to a treatment or target flow rate Qtgt that is typically positive throughout the patient's breathing cycle. Such forms are generally grouped under the heading of flow therapy. In flow therapy, the treatment flow rate Qtgt may be a constant value that is hard-coded or manually entered to the RPT device 4000. If the treatment flow rate Qtgt is sufficient to exceed the patient's peak inspiratory flow rate, the therapy is generally referred to as high flow therapy (HFT). Alternatively, the treatment flow rate may be a profile Qtgt(t) that varies over the respiratory cycle.

### 5.9 AR/VR

A user may experience augmented reality (AR) and/or virtual reality (VR) with the use of a head-mounted display interface. Examples of head-mounted display interfaces are disclosed in WO 2021/137766, WO 2021/189096, U.S. 2021/0302749, and U.S. 2021/0302748, which are each herein incorporated by reference in their entirety.

As shown in FIGS. 54 and 55, a head-mounted display interface 11000 may include a user interface structure 11100, a display unit housing 11200, and a support structure 11300. The head-mounted display interface 11000 may output a computer-generated image to the user wearing the head-mounted display interface 11000.

In some forms, the user interface structure 11100 may be constructed from a comfortable material (e.g., foam, textile, silicone, etc.) and may contact the user's face. The user interface structure 11100 may assist in dispersing the force applied to the user's face so that the head-mounted display interface 11000 is more comfortable to wear.

The display unit housing 11200 may include the electrical components for outputting the computer generated image. The display unit housing 11200 may be formed from a rigid or semi-rigid material in order to protect the electrical components.

The support structure 11300 may be similar to the positioning and stabilising structures described above. For example, the support structure 11300 may include straps constructed at least partially from textile materials. The straps may be able to stretch in order to fit different sized users. The straps may also be rigidized or include a rigidizer in order to provide stiffness and/or stability.

In some forms, the head-mounted display interface 1100 may include a battery (e.g., a rechargeable battery) in the display unit housing 11200. The head-mounted display interface may be removably connected to the charger 6040 in order to recharge the battery.

In other forms, the head-mounted display interface 11000 may include a port (not shown) for receiving a power cord 6020 connected to a battery 6010 (see e.g., FIG. 23).

As illustrated in FIG. 55, some forms of a head-mounted display interface 11000 may include at least one opening 11104 in the user interface structure 11000. When the user dons the head-mounted display interface 11000, the at least one opening 11104 may be aligned with the user's nose. For example, a single opening may align with both nares, or there may be separate openings for each naris. The illustrated example also shows a user interface structure 11000 that cradles the user's nose (e.g., similar to the seal-forming structure 8000 in FIG. 17). Alternatively, the user interface structure 11000 may include a structure around the at least opening 11104 that is received within the user's naris.

In some forms, the display unit housing 11200 may include a blower (e.g., not shown but similar to blower 6502). The blower in the display unit housing 11200 may generate a flow of pressurized breathable gas, which may be output through the at least one opening 11104. The patient may inhale the pressurized gas through their nose as described in any of the examples above. Thus, the user interface structure 11000 may seal around at least a part of the user's face (e.g., to prevent leaks of pressurized air). Additionally, although not illustrated, the opening 11104 may extend around the user's mouth so that the user could also inhale the pressurized air through their mouth.

In some forms, the head-mounted display interface 11000 may combine the features of AR/VR and respiratory therapy. For example, a patient may use the head-mounted display interface 11000 to receive pressurized air in order to alleviate a breathing disorder. Simultaneously, the user may view a computer generated image output from the display unit housing 11200. Utilizing AR/VR with the therapy may make the therapy and wearing a patient interface more comfortable (e.g., thus improving patient compliance). For example, the computer generated image may assist the patient in falling to sleep faster in order to more effectively take advantage of the therapy.

### ALTERNATIVE EXAMPLES

FIGS. 57-79 show a patient interface 12000 combining the features of AR/VR and respiratory therapy according to alternative examples of the present technology. The patient interface 12000 may include a respiratory pressure therapy (RPT) device including an electric blower 12050 configured to generate pressurized breathable air, a head-mounted display interface configured to generate a computer-generated image to the patient, a user interface structure 12100 configured to engage with a patient's face, a flow generator casing 12200 (also referred to as a display unit housing) that at least partly encloses the head-mounted display interface and the electric blower 12050, and a positioning and stabilising structure 12300 (also referred to as a support structure) configured to maintain the user interface structure 12100 in a therapeutically effective position. In some forms, the head-mounted display interface may be in the form of a head-mounted interface configured to substantially cover the eyes of the patient. Such head-mounted interface may not include any display or screen and may not generate any computer-generated image.

The flow generator casing 12200 is configured to support components associated with respiratory therapy (e.g., blower 12050, one or more batteries (e.g., a pair of rechargeable batteries 12070) to power the blower, therapy cushion component 12500) along with components associated with AR/VR (e.g., display interface with electrical components for outputting a computer-generated image, stability cushion component 12700), e.g., see FIGS. 60-64. In an example, the features associated with respiratory therapy may be integrated into existing space (e.g., the casing) occupied by AR/VR features, i.e., synergy between respiratory therapy and AR/VR. Thus, the patient interface 12000 according to an example of the present technology comprises a self-contained CPAP system (e.g., therapy cushion component and RPT device configured to deliver a flow of pressurized breathable air to the patient in a positive pressure range suitable for treatment of a respiratory disorder) along with an AR/VR system (e.g., stability cushion component and display interface) to enhance therapy (video and/or audio output). As noted above, in some forms, the CPAP system may be configured so that the head-mounted interface may not include any display or screen and may not generate any computer-generated image.

### Two-Part Cushion Component

An aspect of one form of the present technology relates to a user interface structure for a patient interface configured to treat a patient with a respiratory disorder, the user interface structure including a stability cushion component and a therapy cushion component that is separate and distinct from the stability cushion component. In this example, the user interface structure 12100 of the interface includes a multi-part construction. For example, the user interface structure 12100 includes a two-part cushion component, i.e., a stability cushion component 12700 and a therapy cushion component 12500 that is separate and distinct from the stability cushion component 12700, e.g., see FIGS. 66-73. The stability cushion component 12700 is configured to removably and interchangeably couple with the flow generator casing 12200, and the therapy cushion component 12500 is configured to removably and interchangeably couple with the stability cushion component 12700 and/or the flow generator casing 12200. This arrangement separates the seal provided by the stability cushion component 12700 from the seal provided by the therapy cushion component 12500 so that they can be independently configured and designed for their specific functions, e.g., stability cushion component 12700 is configured to enhance structure and stability for supporting the weight of the patient interface on the patient's face whereas the therapy cushion component 12500 is configured to enhance seal and comfort for respiratory therapy.

### Stability Cushion Component

An aspect of one form of the present technology relates to a stability cushion component for a patient interface configured to treat a patient with a respiratory disorder, the stability cushion component configured to extend about a periphery of the patient's eyes. In an example, the stability cushion component 12700 is configured to extend about a periphery of the display interface (e.g., VR display) contained by the flow generator casing 12200, and form a viewing opening to the display interface. In use, the stability cushion component 12700 extends around a periphery of the user's eyes, and may engage with the user's face (e.g., along the user's cheek and/or forehead) to provide substantially complete sealing around the user's eyes (e.g., to at least partially block light from entering the flow generator casing 12200 in order to facilitate immersion in the use of the display interface). Moreover, the stability cushion component 12700 is configured and arranged to support the patient interface 12000 on the user's face in a comfortable stable manner.

The stability cushion component 12700 includes a chassis 12750 and a cushion 12775 configured to engage with the user's face. The chassis 12750 is relatively harder than the cushion 12775, e.g., the chassis comprises polycarbonate and the cushion comprises foam and/or silicone or other suitable materials.

The chassis 12750 is configured and arranged to repeatably engage with and disengage from the flow generator casing 12200. In an example, the chassis 12750 may comprise one or more retainer parts (e.g., clips) around its periphery configured to repeatably and removably engage corresponding retainer parts provided to the flow generator casing 12200 when the stability cushion component 12700 is coupled with the flow generator casing 12200. In an example, the retainer parts are substantially rigid such that engagement between the retainer parts provides a hard-to-hard connection.

The chassis 12750 acts as a base for the stability cushion component 12700, and may at least partially act as a base for the therapy cushion component 12500, e.g., when the therapy cushion component 12500 is coupled with the stability cushion component 12700. In addition, the chassis 12750 may provide some rigidity and necessary structure to the stability cushion component 12700, and may also at least partially provide some rigidity and necessary structure to the therapy cushion component 12500.

In an example, the cushion 12775 comprises a face contacting portion configured to engage with the user's face and a non-face contacting portion configured to connect the cushion 12775 to the chassis 12750.

In one example, e.g., see FIGS. 74 and 75, the non-face contacting portion includes a side wall 12780 provided to the chassis 12750 and a ledge 12785 configured to project inwardly from the side wall 12780 in a cantilever manner towards the viewing opening. The face contacting portion comprises a membrane 12790 including a face engaging surface configured to contact the user's face around a periphery of the user's eyes. The face contacting portion also comprises a foam cushion 12795. As illustrated, the membrane 12790 forms a loop so that the membrane 12790 and the face engaging surface thereof covers the foam cushion 12795 such that the foam cushion 12795 is arranged underneath the face engaging surface and enclosed by the membrane 12790. The non-face contacting portion is configured to resiliently support the foam cushion and the membrane such that it applies a reactive force when in use on a user's face, e.g., the ledge 12785 is configured to provide a resilient, spring-like support to the foam cushion 12795 and the membrane 12790. In an example, the side wall 12780, the ledge 12785, and the membrane 12790 comprise an elastomeric material, e.g., silicone.

In another example, e.g., see FIGS. 76 and 77, the non-face contacting portion includes a side wall 12780 provided to the chassis 12750 and a ledge 12785 configured to project outwardly from the side wall 12780 in a cantilever manner away from the viewing opening. The face contacting portion comprises a foam cushion 12795 including a face engaging surface configured to contact the user's face around a periphery of the user's eyes. The non-face contacting portion is configured to resiliently support the foam cushion such that it applies a reactive force when in use on a user's face, e.g., the ledge 12785 is configured to provide a resilient, spring-like support to the foam cushion 12795. In an example, the side wall 12780 and the ledge 12785 comprise an elastomeric material, e.g., silicone.

In an example, the foam cushion 12795 (e.g., polyurethane foam or viscoelastic foam) may be at least partially compressed against the user's face (e.g., via the non-face contacting portion) to conform to the contours of the user's face and to support at least a portion of the weight of the patient interface. The foam cushion may be configured and arranged such that the compression force or load applied to the user's face is distributed or spread around a periphery thereof so that the load is not concentrated on a minimal number of contact points. Moreover, the foam cushion may comprise varying compliance (e.g., varied rigidity or density) around the periphery thereof configured to allow selective distribution of the force onto the user's face. This arrangement allows higher levels of pressure to be spread across regions of the user's face that are more adept at absorbing the pressure, e.g., epicranius and the sphenoid.

In an example, the foam cushion is configured to provide comfort whereas the non-face contacting portion provides a resilient support structure (e.g., bellows-type structure with at least one fold to provide stabilizing and load bearing properties).

In an example, e.g., see FIGS. 76-77, the non-face contacting portion is configured to form an outward concave curvature which encourages outward bending of the cushion in use, e.g., foam cushion 12795 oriented so that the face engaging surface deflects away from the viewing opening when in use.

In an example, one or more sensors may be provided to the stability cushion component (e.g., adjacent the forehead) to detect degeneration of the component to suggest replacement and/or provide a reminder to clean the component.

### Therapy Cushion Component

An aspect of one form of the present technology relates to a therapy cushion component for a patient interface configured to treat a patient with a respiratory disorder, the therapy cushion component is configured to form a plenum chamber and form a seal with the patient's nose and/or mouth. In an example, the therapy cushion component 12500 is configured to form a seal with the user's nose and/or mouth, and to deliver the flow of pressurized breathable air from the blower 12050 to the patient's nose and/or mouth. The therapy cushion component 12500 may repeatably and removably engage with the stability cushion component 12700 and/or the flow generator casing 12200.

In an example, the therapy cushion component 12500 includes a front portion 12550 and a cushion 12575 (also referred to as a seal-forming structure) configured to form a seal against the patient's face, e.g., with the patient's nose and/or mouth. The front portion 12550 and the cushion 12575 form at least a portion of an interior breathing chamber (plenum chamber) configured to receive the pressurized air. The front portion 12550 is relatively harder than the cushion 12575 (e.g., the front portion comprises polycarbonate and the cushion comprises silicone), and the front portion 12550 forms an opening configured to communicate with the flow generator casing and the flow of pressurized gas from the blower.

The front portion 12550 may be permanently (e.g., co-molded, overmolded) or removably (e.g., mechanical interlock) connected to the cushion 12575. In an example, the cushion is constructed of a relatively flexible or pliable material (e.g., silicone) and the front portion is constructed of a relatively rigid material (e.g., polycarbonate).

The front portion 12550 and the cushion 12575 of the therapy cushion component may form a unit that as a whole is interchangeable with the stability cushion component 12700 and/or the flow generator casing 12200. That is, the therapy cushion component 12500 is repeatedly engageable with and removably disengageable from the stability cushion component 12700 and/or the flow generator casing 12200.

In an example, the stability cushion component 12700 may be provided in one size and/or type (e.g., one size fits most), which may be selectively utilized with multiple sizes and/or types of the therapy cushion component 12500. The therapy cushion component 12500 may be provided in two or more sizes and/or types (e.g., small, medium and large sizes, silicone and/or foam cushions, etc.) that include at least one aspect different from one another (e.g., different heights and/or widths of the cushion, different materials of the cushion). In alternative examples, the stability cushion component 12700 may also be provided with multiple sizes and/or types (e.g., silicone and/or foam cushions).

Each front portion 12550 of the different size/type therapy cushion components 12500 includes a retainer part or interface that is common or similar for all sizes/types, which allows the different size/type therapy cushion components 12500 to be connected or interfaced with the common stability component and/or the flow generator casing. For example, the one size or common stability cushion component may be connected or interfaced with each of the different size/type therapy cushion components, i.e., each therapy cushion component includes common mating geometry for all sizes/types.

In an example, the cushion 12575 of the therapy cushion component 12500 comprises a nasal cushion including a flexible membrane 12580 configured to seal around the patient's nose, and sealing forces (force vectors) are arranged to extend substantially horizonal (e.g., substantially parallel to the Frankfort horizontal plane). That is, the nasal cushion is pulled (via the positioning and stabilising structure 12300) into the user's face under a suitable compressive force that is substantially in line with the Frankfort horizontal plane (pulled directly back into the user's face). Such force vectors substantially align with the force vectors associated with the stability cushion component 12700 which are also substantially in line with the Frankfort horizontal plane. This arrangement may simplify use and adjustment as forces vectors for both the therapy cushion component 12500 and the stability cushion component 12700 are in the same plane, e.g., the positioning and stabilising structure 12300 may simply include side straps arranged in the Frankfort horizontal plane.

In an example, the therapy cushion component 12500 and the stability cushion component 12700 may comprise different compression rates, e.g., therapy cushion may be softer or more compliant than the stability cushion to readily deform to contours of the patient's face for sealing during therapy.

### Connection of Therapy Cushion Component to Stability Cushion Component

In an example, e.g., see FIGS. 72 and 73, the therapy cushion component 12500 and the stability cushion component 12700 include cooperating retaining structures to repeatably and removably connect the therapy cushion component 12500 to the stability cushion component 12700. In an example, the therapy cushion component 12500 is releasably connectable to the stability cushion component 12700 to facilitate replacement and/or cleaning, and to allow alternative therapy cushion components 12500 and stability cushion components 12700 to be connected to one another. Such arrangement allows multiple therapy cushion components 12500 (e.g., sizes and/or types) to be used with the stability cushion component 12700 and therefore provide a user interface structure 12100 suitable for most users.

In an example, the chassis 12750 of the stability cushion component 12700 (e.g., constructed of a relatively hard plastic material such as polycarbonate) provides an interface with the therapy cushion component 12500. For example, the front portion 12550 of the therapy cushion component 12500 includes a retainer part configured and arranged to repeatably and removably engage a retainer part provided to the chassis 12750 of the stability cushion component 12700 when the therapy cushion component 12500 is coupled with the stability cushion component 12700. In an example, the retainer parts are substantially rigid such that engagement between the retainer parts provides a hard-to-hard connection, e.g., via a snap-fit connection (e.g., repeatably engagable and disengageble).

In an example, the perimeter and/or shape of the front portion of the therapy cushion component is configured to facilitate alignment of the front portion to the chassis of the stability cushion component for engagement. For example, the shape of the front portion may be asymmetrical to minimize misalignment.

In an example, when the therapy cushion component and the stability cushion component are engaged, accidental disengagement is prevented to the structure or geometry of the retainer parts, i.e., the retainer parts are configured to maintain engagement during use and prevent any unintentional or partial disassembly during use.

In an example, the therapy cushion component is coupled with the stability cushion component to provide a substantially non-adjustable assembly in which adjustable movement between the therapy cushion component and the stability cushion component is substantially resisted.

### Manifold

In an example, a manifold 12400 (e.g., constructed of a relatively hard plastic material such as polycarbonate) is provided to the chassis 12750 (e.g., integrally molded) of the stability cushion component 12700, and the manifold 12400 provides the interface or retaining structure with the therapy cushion component 12500, e.g., see FIGS. 72 and 73. The manifold 12400 is hollow and is configured to form an airflow path to assist in conveying the flow of pressurized breathable air from the blower 12050 to the interior breathing chamber of the therapy cushion component 12500.

As illustrated, the manifold 12400 includes a first opening 12410 and a second opening 12420 that is oriented substantially perpendicular to the first opening 12410, which allows the manifold to change the direction of the airflow. For example, the airflow path may travel in the inferior direction through the first opening before traveling in a posterior direction through the second opening.

The first opening 12410 is configured to communicate with the interior of the flow generator casing 12200 and the blower 12050 thereof, and the second opening 12420 is configured to communicate with the therapy cushion component 12500, e.g., see FIG. 75. A retention flange bounds the second opening 12420 and forms at least part of a retainer part configured to repeatably and removably engage a retainer part provided to the front portion 12550 of the therapy cushion component 12500 when the therapy cushion component 12500 is coupled with the stability cushion component 12700.

In one form, the therapy cushion component 12500 may be engageable/disengageable with the stability cushion component 12700 by moving the therapy cushion component 12500 towards the stability cushion component 12700 in a substantially anterior-posterior direction, e.g., see FIG. 73. However, engagement/disengagement may comprise alternative directions, e.g., depending on the retaining structures.

When the therapy cushion component 12500 is coupled with the stability cushion component 12700, the therapy cushion component 12500 and the stability cushion component 12700 may form a unit that as a whole is interchangeable with the flow generator casing 12200, e.g., see FIG. 72. That is, the stability cushion component 12700 and the therapy cushion component 12500 connected thereto is repeatedly engageable with and removably disengageable from the flow generator casing 12200.

In addition, when the stability cushion component 12700 is coupled with the flow generator casing 12200, the therapy cushion component 12500 may be repeatably engageable with and removably disengageable from the stability cushion component 12700, e.g., to allow removal of the therapy cushion component 12500 while the stability cushion component 12700 remains connected to the flow generator casing 12200.

In another example, the manifold 12400 may form a separate and distinct structure from the stability cushion component 12700. In this example, the manifold 12400 may be releasably coupled to the therapy cushion component 12500, and therefore the manifold 12400 and the therapy cushion component 12500 may form a unit that as a whole is interchangeable with the flow generator casing 12200 and/or the stability cushion component 12700, e.g., see FIGS. 67-71. For example, the manifold 12400 may include cooperating retaining structures to repeatably and removably connect the manifold 12400 (along with the therapy cushion component 12500) to the flow generator casing 12200 and/or the stability cushion component 12700.

In an example, the manifold 12400 (along with the therapy cushion component 12500) and the stability cushion component 12700 may be repeatedly and removably engageable with the flow generator casing 12000 independent from one another.

In one form, the stability cushion component 12700 may be engageable/disengageable with the flow generator casing 12000 by moving the stability cushion component 12700 towards the flow generator casing 12000 in a substantially anterior-posterior direction, and the therapy cushion component 12500 may be engageable/disengageable with the flow generator casing 12000 by moving the therapy cushion component 12500 towards the flow generator casing 12000 in a substantially superior-inferior direction, e.g., see FIG. 70. That is, the assembly directions for the stability cushion component 12700 and the therapy cushion component 12500 may be different from one another, e.g., transverse. However, engagement/disengagement may comprise alternative directions, e.g., depending on the retaining structures.

### EAV

In an example, an expiratory activated valve (EAV) 12800 is arranged adjacent to the manifold 12400 and the therapy cushion component 12500 to control airflow to the therapy cushion component 12500, e.g., see FIGS. 71, 73, and 75. As described above, the EAV 12800 includes a one-way duck bill valve configured to open during patient inspiration to direct pressurized gas from the blower 12050 to the breathing chamber of the therapy cushion component 12500, and close during patient exhalation to prevent reverse airflow back towards the blower 12050 while the EAV 12800 introduces an airflow path from the breathing chamber to atmosphere for venting. It should be appreciated that other types of valves may replace the EAV without departing from the scope of the examples.

In an example, as shown in FIGS. 71, 73, and 75, the manifold 12400 and the front portion 12550 of the therapy cushion component 12500 cooperate to house the EAV 12800, e.g., each of the manifold and the front portion at least partially receives, locates and/or retains a portion of the EAV in position.

In the illustrated examples, the therapy cushion component 12500 is repeatably engageable with and removably disengageable from the manifold 12400, which allows removal of the EAV 12800 for replacement and/or cleaning.

In one form, the EAV 12800 may be integrated with the therapy cushion component 12500 (e.g., the EAV 12800, manifold 12400, and therapy cushion component 12500 form a unit) so that the EAV 12800 and the therapy cushion component 12500 may be replaceable as a unit, e.g., see FIGS. 67-71.

### Flow Generator Casing

In an example, e.g., see FIGS. 65-69, the flow generator casing 12200 includes a front case 12210 and a rear case 12220 that cooperate to house and support components for respiratory therapy and AR/VR. The flow generator casing 12200 may also include a shroud 12230, e.g., to hide interfaces between the front case/rear case, provide connection to the positioning and stabilising structure, provide air inlet openings, provide air outlet openings, house inlet filter.

In an example the rear case 12220 includes a two-part construction, i.e., a first part 12221 and a second part 12222 connected to the first part 12221.

In an example, the posterior side of the first part 12221 of the rear case comprises one or more retainer parts (e.g., clips) around its periphery configured to repeatably and removably engage with the corresponding retainer parts provided to the chassis 12770 of the stability cushion component 12700 when the stability cushion component 12700 is coupled with the flow generator casing 12200.

In an example, an inferior portion of the first part 12221 is configured to at least partially receive, locate and/or retain a portion of the therapy cushion component 12500 and/or manifold 12400. For example, the inferior portion of the first part 12221 may form a recess with cooperating retaining structures to repeatably and removably connect the manifold 12400 (along with the therapy cushion component 12500) to the flow generator casing 12200.

The second part 12222 of the rear case forms a cavity configured to at least partially receive, locate and/or retain the blower 12050. In an example, a superior portion of the cavity is configured to retain the blower 12050, and an inferior portion of the cavity is communicated with a central outlet of the blower 12050. When connected, the second part 12222 cooperates with the first part 12221 to form a volute configured to convey the flow of pressurized breathable air from the blower 12050 to the manifold 12400 (which then conveys the flow of pressurized breathable air to the therapy cushion component 12500), e.g., see FIGS. 74-75.

As described above, the blower 12050 includes a substantially cylindrical shape and forms a parallel stage arrangement, wherein the blower 12050 includes at least a pair of impellers coupled to a common shaft and arranged in parallel. That is, the blower 12050 includes an inlet with a corresponding impeller on each side of the blower that will produce parallel flows of air through the blower that will combine to achieve a desired flow at the central outlet.

In an example, a suspension may be provided to receive and resiliently support the blower 12050 within the rear case 12220. In an example, the second part 12222 includes retaining walls with respective openings configured to align with respective ones of the inlets of the blower 12050.

In an example, the second part 12222 of the rear case is configured to at least partially receive, locate and/or retain a portion of a battery, e.g., to power the blower and/or electronics of the AR/VR. For example, in the illustrated example, the second part 12222 is configured to retain two batteries 12070, i.e., one battery 12070 arranged on each side of the blower 12050. As illustrated, each battery 12070 includes a substantially cylindrical shape, and each side of the second part 12222 may include a rounded retaining structure configured to retain respective ones of the batteries 12070.

The front case 12210 is connected (e.g., removably connected) to the rear case 12220 so that the front case 12221 encloses the second part 12222 and substantially covers the anterior side of the first part 12221. As a result, the front case and the rear case cooperate to house and retain the blower, batteries, display interface, and electronics associated with the blower and display interface.

In an example, the front case, the rear case and/or the shroud may include one or more inlet openings to receive ambient air for delivery to the blower. For example, FIGS. 78-79 show an example in which inlet openings 12231 are provided to the shroud 12230. In an example, such intake airflow may at least partially pass along and/or through the user interface structure to at least partially cool the same, which cooling may have a beneficial effect for sleep.

In an example, the intake airflow and/or pressurized airflow from the blower may have alternative uses than respiratory therapy, e.g., use airflow for cooling the cushions, immersive gaming, aromatherapy.

In an example, the front case and/or the rear case may include one or more outlet openings (vent openings) to allow exhaust to ambient from respiratory therapy. In an example, the front case and/or the rear case may form an exhaust channel communicated with the EAV to allow exhaled air to pass around the EAV, into the exhaust channel, and through the one or more vent openings.

In an example, one or more pressure sensors may be supported within the flow generator casing 12200 to monitor pressure associated with respiratory therapy, e.g., adjacent the blower, adjacent the therapy cushion component.

### Positioning and Stabilising Structure

In an example, the positioning and stabilising structure 12300 may be adjustably and removably connected to at least a portion of the flow generator casing 12200 to hold the patient interface 12000 in an operable position on the patient's face.

In an example, as shown in FIGS. 57-59, the positioning and stabilising structure 12300 may include a single side strap 12310 configured to extend along the sides of the patient's head and pass over respective ears, e.g., to facilitate donning and adjustment. The side strap 12310 may include ends that adjustably connect (via a mechanical fastener, hook and look material, magnets, etc.) to respective sides of the flow generator casing 12200. In an example, each side of the flow generator casing 12200 may include a strap connector, e.g., cross-bar that forms a strap opening that respective ends of the side strap may be threaded through.

In an example, one or more strap connectors for the positioning and stabilising structure 12300 may be provided to the front case 12210, the rear case 12220 and/or the shroud 12230 of the flow generator casing 12200.

### Two-Part Cushion for Full-Face Patient Interface

In an example, a two-part cushion construction may be applicable to the seal-forming structure of the full-face patient interface for respiratory therapy described above, e.g., the seal-forming structure 6100 in FIG. 31.

For example, as shown in FIGS. 80 to 83, the seal-forming structure 6100 may include a two-part cushion component, i.e., a nasal cushion component 6110 and a mouth cushion component 6120 that is separate and distinct from the nasal cushion component 6110. The nasal cushion component 6110 is configured to removably and interchangeably couple with the casing 6408, and the mouth cushion component 6120 is configured to removably and interchangeably couple with the casing 6408 independent from the nasal cushion component 6110. This arrangement separates the seal provided by the nasal cushion component 6110 from the seal provided by the mouth cushion component 6120 so that they can be independently configured and selected for their specific functions, e.g., mouth cushion component is configured to enhance structure and stability for supporting the weight of the interface on the patient's face whereas the nasal cushion component is configured to enhance seal and comfort for respiratory therapy.

In an example, the nasal cushion component 6110 is configured to form a seal with the user's nose (i.e., around the patient's nares), and to deliver the flow of pressurized breathable air from the blower to the patient's nose. For example, the nasal cushion component 6110 may seal around the patient's alar rims while avoiding contact with the patient's nasal ridge.

In an example, the mouth cushion component 6120 may comprise a foam cushion arrangement similar to that described above in relation to the stability cushion component 12700, e.g., the mouth cushion component 6120 includes a foam cushion resiliently supported by a non-face contacting portion. In an example, the mouth cushion component may be configured to form a seal around at least a portion of the patient's mouth, e.g., nasal cushion component configured to form a seal along the patient's upper lip/upper portion of the patient's mouth.

In an example, the patient interface is configured such that pressurized breathable air from the blower is only delivered to the patient's nose via the nasal cushion component 6110. In an alternative example, the patient interface may be configured such that pressurized breathable air is delivered to both the patient's nose and mouth via both the nasal cushion component and the mouth cushion component.

Such two-part arrangement allows different size/type nasal cushion components and/or mouth cushion components to be connected with the casing, and allows removal of the nasal cushion component and/or the mouth cushion component for replacement and/or cleaning.

In an alternative example, the nasal cushion component may be releasably coupled to the mouth cushion component, and therefore the nasal cushion component and the mouth cushion component may form a unit that as a whole is interchangeable with the casing.

### 5.10 Audio

As shown in FIG. 56, an audio system 6800 may be used with a patient interface. For example, the patient interface 8000 is shown, but the audio system 6800 may be used with any of the patient interfaces. Additionally, the audio system 6800 may also be used with the head-mounted display interface 11000.

In some forms, the patient interface 8000 may include an alternate positioning and stabilising structure 8375, which may include a front strap 8376 that may be similar to the front strap 6304. For example, the illustrated front strap 8376 may connect (e.g., either permanently or removably) to the flow generator casing 6400. The positioning and stabilising structure 8375 is illustrated with the audio system 6800, but may also be used without the audio system 6800.

In certain forms, the positioning and stabilising 8376 may include a hoop 8378 connected to the front strap 8376. The hoop 8378 may extend around the patient's head from the patient's frontal bone to the patient's occipital bone. In the illustrated example, the hoop 8378 may be a continuous piece of material, although in other examples, the hoop 8378 may include multiple pieces that allow for adjustment of the length of the hoop 8378.

In some forms, the front strap 8376 may be adjustable relative to the hoop 8378 so that patient may change the angle of the front strap 8376 relative to the hoop 8378 (e.g., so that the patient can find a comfortable fit).

In some forms, an audio system 6800 may be connected to the positioning and stabilising structure 8375. For example, the audio system 6800 may be connected to the hoop 8378. The audio system 6800 may be positioned on an interior of the hoop 8378 so that the patient wearing the patient interface 8000 may contact the audio system 6800.

In some forms, the audio system 6800 includes a pair of output devices 6804. Each output device 6804 may output sound to one of the patient's ears. In the illustrated example, the output devices 6804 are formed as earmuffs and may rest against and/or enclose each of the patient's ears. In other examples (not shown), the output devices 6804 may be earbuds that fit within the patient's ears.

In certain forms, the output devices 6804 may output noise to the patient using the patient interface 8000. For example, the patient may play music, white noise, or any desired sound. The sound output from the output device 6804 may assist the patient to relax while using the patient interface 8000 (e.g., in order to fall asleep quicker and stay asleep).

In certain forms, a user may use the audio system 6800 without the patient interface 8000. For example, International Application No. PCT/SG2021/050590 describes an example without a patient interface for providing pressurized air, and is incorporated herein by reference in its entirety.

In some forms, the power cord 6020 may be connected to the front strap 8376 of the positioning and stabilising structure 8375. In other examples, the power cord 6020 may be connected to the flow generator casing 8400, or the patient interface 8000 may include a removable battery (e.g., battery 6030 or battery 6035).

### 5.11 Glossary

For the purposes of the present technology disclosure, in certain forms of the present technology, one or more of the following definitions may apply. In other forms of the present technology, alternative definitions may apply.

### 5.11.1 General

Air: In certain forms of the present technology, air may be taken to mean atmospheric air, and in other forms of the present technology air may be taken to mean some other combination of breathable gases, e.g. oxygen enriched air.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

In the example of patient respiration, a flow rate may be nominally positive for the inspiratory portion of a breathing cycle of a patient, and hence negative for the expiratory portion of the breathing cycle of a patient. Device flow rate, Qd, is the flow rate of air leaving the RPT device. Total flow rate, Qt, is the flow rate of air and any supplementary gas reaching the patient interface via the air circuit. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient's respiratory system.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Oxygen enriched air: Air with a concentration of oxygen greater than that of atmospheric air (21%), for example at least about 50% oxygen, at least about 60% oxygen, at least about 70% oxygen, at least about 80% oxygen, at least about 90% oxygen, at least about 95% oxygen, at least about 98% oxygen, or at least about 99% oxygen. “Oxygen enriched air” is sometimes shortened to “oxygen”.

Medical Oxygen: Medical oxygen is defined as oxygen enriched air with an oxygen concentration of 80% or greater.

Patient: A person, whether or not they are suffering from a respiratory condition.

Pressure: Force per unit area. Pressure may be expressed in a range of units, including cmH₂O, g-f/cm² and hectopascal. 1 cmH₂O is equal to 1 g-f/cm² and is approximately 0.98 hectopascal (1 hectopascal=100 Pa=100 N/m²=1 millibar~0.001 atm). In this specification, unless otherwise stated, pressure is given in units of cmH₂O.

The pressure in the patient interface is given the symbol Pm, while the treatment pressure, which represents a target value to be achieved by the interface pressure Pm at the current instant of time, is given the symbol Pt.

Respiratory Pressure Therapy: The application of a supply of air to an entrance to the airways at a treatment pressure that is typically positive with respect to atmosphere.

Ventilator: A mechanical device that provides pressure support to a patient to perform some or all of the work of breathing.

5.11.1.1 Materials & their Properties

(Durometer Hardness (Indentation Hardness): A material property measured by indentation of an indentor (e.g. As measured in accordance with ASTM D2240).
‘Soft’ materials may include silicone or thermo-plastic elastomer (TPE), and may, e.g. readily deform under finger pressure.
‘Hard’ materials may include polycarbonate, polypropylene, steel or aluminium, and may not e.g. readily deform under finger pressure.

Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45.

Polycarbonate: a thermoplastic polymer of Bisphenol-A Carbonate.

### 5.11.1.2 Mechanics

### Axes:

a. Neutral axis: An axis in the cross-section of a beam or plate along which there are no longitudinal stresses or strains.

Deformation: The process where the original geometry of a member (structure or component) changes when subjected to forces, e.g. a force in a direction with respect to an axis. The process may include stretching or compressing, bending and twisting.

Stiffness: The ability of a structure or component to resist deformation in response to an applied load. A structure or component may have an axial stiffness, a bending stiffness, and a torsional stiffness. A structure or component is said to be stiff when it does not deform easily when subject to mechanical forces. Stiffness of a structure or component is related to its material properties and its shape. The inverse of stiffness is flexibility.

Elasticity: The ability of a material to return to its original geometry after deformation.

Viscous: The ability of a material to resist flow.

Visco-elasticity: The ability of a material to display both elastic and viscous behaviour in deformation.

Yield: The situation when a material can no longer return back to its original geometry after deformation.

### 5.11.1.3 Structural Elements

Thin Structures:
   a. Beams,
   b. Membranes, Plates & Shells
Thick structures: Solids

Shell: A shell will be taken to mean a curved, relatively thin structure having bending, tensile and compressive stiffness. For example, a curved structural wall of a mask may be a shell. In some forms, a shell may be faceted. In some forms a shell may be airtight. In some forms a shell may not be airtight.

Membrane: Membrane will be taken to mean a typically thin element that has, preferably, substantially no resistance to bending, but has resistance to being stretched.

Load transfer member: A structural member which transfers load from one location to another.

Load support member: A structural member which transfers load from one location to a non-structural item, such as the face.

Tension member: A structural element that is subjected to tensional forces.

Tie (noun): A structure designed to resist tension.

Compression member: A structural element that is subjected to compression forces.

Strut: A strut will be taken to be a structural component designed to increase the compression resistance of another component in at least one direction.

Stiffener: A stiffener will be taken to mean a structural component designed to increase the bending resistance of another component in at least one direction.

Elbow: An elbow is an example of a structure that directs an axis of flow of air travelling therethrough to change direction through an angle. In one form, the angle may be approximately 90 degrees. In another form, the angle may be more, or less than 90 degrees. The elbow may have an approximately circular cross-section. In another form the elbow may have an oval or a rectangular cross-section. In certain forms an elbow may be rotatable with respect to a mating component, e.g. about 360 degrees. In certain forms an elbow may be removable from a mating component, e.g. via a snap connection. In certain forms, an elbow may be assembled to a mating component via a one-time snap during manufacture, but not removable by a patient.

Frame: Frame will be taken to mean a mask structure that bears the load of tension between two or more points of connection with a headgear. A mask frame may be a non-airtight load bearing structure in the mask. However, some forms of mask frame may also be air-tight.

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Swivel (noun): A subassembly of components configured to rotate about a common axis, preferably independently, preferably under low torque. In one form, the swivel may be constructed to rotate through an angle of at least 360 degrees. In another form, the swivel may be constructed to rotate through an angle less than 360 degrees. When used in the context of an air delivery conduit, the sub-assembly of components preferably comprises a matched pair of cylindrical conduits. There may be little or no leak flow of air from the swivel in use.

### 5.11.2 Respiratory Cycle

Apnea: According to some definitions, an apnea is said to have occurred when flow falls below a predetermined threshold for a duration, e.g. 10 seconds. An obstructive apnea will be said to have occurred when, despite patient effort, some obstruction of the airway does not allow air to flow. A central apnea will be said to have occurred when an apnea is detected that is due to a reduction in breathing effort, or the absence of breathing effort, despite the airway being patent. A mixed apnea occurs when a reduction or absence of breathing effort coincides with an obstructed airway.

Breathing rate: The rate of spontaneous respiration of a patient, usually measured in breaths per minute.

Duty cycle: The ratio of inhalation time, Ti to total breath time, Ttot.

Effort (breathing): The work done by a spontaneously breathing person attempting to breathe.

Expiratory portion of a breathing cycle: The period from the start of expiratory flow to the start of inspiratory flow.

Flow limitation: Flow limitation will be taken to be the state of affairs in a patient's respiration where an increase in effort by the patient does not give rise to a corresponding increase in flow. Where flow limitation occurs during an inspiratory portion of the breathing cycle it may be described as inspiratory flow limitation. Where flow limitation occurs during an expiratory portion of the breathing cycle it may be described as expiratory flow limitation.

Types of flow limited inspiratory waveforms:
(i) Flattened: Having a rise followed by a relatively flat portion, followed by a fall.
(ii) M-shaped: Having two local peaks, one at the leading edge, and one at the trailing edge, and a relatively flat portion between the two peaks.
(iii) Chair-shaped: Having a single local peak, the peak being at the leading edge, followed by a relatively flat portion.
(iv) Reverse-chair shaped: Having a relatively flat portion followed by single local peak, the peak being at the trailing edge.

Hypopnea: According to some definitions, a hypopnea is taken to be a reduction in flow, but not a cessation of flow. In one form, a hypopnea may be said to have occurred when there is a reduction in flow below a threshold rate for a duration. A central hypopnea will be said to have occurred when a hypopnea is detected that is due to a reduction in breathing effort. In one form in adults, either of the following may be regarded as being hypopneas:
(i) a 30% reduction in patient breathing for at least 10 seconds plus an associated 4% desaturation; or
(ii) a reduction in patient breathing (but less than 50%) for at least 10 seconds, with an associated desaturation of at least 3% or an arousal.

Hyperpnea: An increase in flow to a level higher than normal.

Inspiratory portion of a breathing cycle: The period from the start of inspiratory flow to the start of expiratory flow will be taken to be the inspiratory portion of a breathing cycle.

Patency (airway): The degree of the airway being open, or the extent to which the airway is open. A patent airway is open. Airway patency may be quantified, for example with a value of one (1) being patent, and a value of zero (0), being closed (obstructed).

Positive End-Expiratory Pressure (PEEP): The pressure above atmosphere in the lungs that exists at the end of expiration.

Peak flow rate (Qpeak): The maximum value of flow rate during the inspiratory portion of the respiratory flow waveform.

Respiratory flow rate, patient airflow rate, respiratory airflow rate (Qr): These terms may be understood to refer to the RPT device's estimate of respiratory flow rate, as opposed to “true respiratory flow rate” or “true respiratory flow rate”, which is the actual respiratory flow rate experienced by the patient, usually expressed in litres per minute.

Tidal volume (Vt): The volume of air inhaled or exhaled during normal breathing, when extra effort is not applied. In principle the inspiratory volume Vi (the volume of air inhaled) is equal to the expiratory volume Ve (the volume of air exhaled), and therefore a single tidal volume Vt may be defined as equal to either quantity. In practice the tidal volume Vt is estimated as some combination, e.g. the mean, of the inspiratory volume Vi and the expiratory volume Ve.

Inhalation Time (Ti): The duration of the inspiratory portion of the respiratory flow rate waveform.

Exhalation Time (Te): The duration of the expiratory portion of the respiratory flow rate waveform.

Total Time (Ttot): The total duration between the start of one inspiratory portion of a respiratory flow rate waveform and the start of the following inspiratory portion of the respiratory flow rate waveform.

Typical recent ventilation: The value of ventilation around which recent values of ventilation Vent over some predetermined timescale tend to cluster, that is, a measure of the central tendency of the recent values of ventilation.

Upper airway obstruction (UAO): includes both partial and total upper airway obstruction. This may be associated with a state of flow limitation, in which the flow rate increases only slightly or may even decrease as the pressure difference across the upper airway increases (Starling resistor behaviour).

Ventilation (Vent): A measure of a rate of gas being exchanged by the patient's respiratory system. Measures of ventilation may include one or both of inspiratory and expiratory flow, per unit time. When expressed as a volume per minute, this quantity is often referred to as “minute ventilation”. Minute ventilation is sometimes given simply as a volume, understood to be the volume per minute.

### 5.11.3 Ventilation

Adaptive Servo-Ventilator (ASV): A servo-ventilator that has a changeable, rather than fixed target ventilation. The changeable target ventilation may be learned from some characteristic of the patient, for example, a respiratory characteristic of the patient.

Backup rate: A parameter of a ventilator that establishes the minimum breathing rate (typically in number of breaths per minute) that the ventilator will deliver to the patient, if not triggered by spontaneous respiratory effort.

Cycled: The termination of a ventilator's inspiratory phase. When a ventilator delivers a breath to a spontaneously breathing patient, at the end of the inspiratory portion of the breathing cycle, the ventilator is said to be cycled to stop delivering the breath.

Expiratory positive airway pressure (EPAP): a base pressure, to which a pressure varying within the breath is added to produce the desired interface pressure which the ventilator will attempt to achieve at a given time.

End expiratory pressure (EEP): Desired interface pressure which the ventilator will attempt to achieve at the end of the expiratory portion of the breath. If the pressure waveform template Π(Φ) is zero-valued at the end of expiration, i.e. Π(Φ)=0 when Φ=1, the EEP is equal to the EPAP.

Inspiratory positive airway pressure (IPAP): Maximum desired interface pressure which the ventilator will attempt to achieve during the inspiratory portion of the breath.

Pressure support: A number that is indicative of the increase in pressure during ventilator inspiration over that during ventilator expiration, and generally means the difference in pressure between the maximum value during inspiration and the base pressure (e.g., PS=IPAP−EPAP). In some contexts, pressure support means the difference which the ventilator aims to achieve, rather than what it actually achieves.

Servo-ventilator: A ventilator that measures patient ventilation, has a target ventilation, and which adjusts the level of pressure support to bring the patient ventilation towards the target ventilation.

Spontaneous/Timed (SIT): A mode of a ventilator or other device that attempts to detect the initiation of a breath of a spontaneously breathing patient. If however, the device is unable to detect a breath within a predetermined period of time, the device will automatically initiate delivery of the breath.

Swing: Equivalent term to pressure support.

Triggered: When a ventilator, or other respiratory therapy device such as an RPT device or portable oxygen concentrator, delivers a volume of breathable gas to a spontaneously breathing patient, it is said to be triggered to do so. Triggering usually takes place at or near the initiation of the respiratory portion of the breathing cycle by the patient's efforts.

### 5.11.4 Anatomy

### 5.11.4.1 Anatomy of the Face

Ala: the external outer wall or “wing” of each nostril (plural: alar)

Alare: The most lateral point on the nasal ala.

Alar curvature (or alar crest) point: The most posterior point in the curved base line of each ala, found in the crease formed by the union of the ala with the cheek.

Auricle: The whole external visible part of the ear.

(nose) Bony framework: The bony framework of the nose comprises the nasal bones, the frontal process of the maxillae and the nasal part of the frontal bone.

(nose) Cartilaginous framework: The cartilaginous framework of the nose comprises the septal, lateral, major and minor cartilages.

Columella: the strip of skin that separates the nares and which runs from the pronasale to the upper lip.

Columella angle: The angle between the line drawn through the midpoint of the nostril aperture and a line drawn perpendicular to the Frankfort horizontal while intersecting subnasale.

Frankfort horizontal plane: A line extending from the most inferior point of the orbital margin to the left tragion. The tragion is the deepest point in the notch superior to the tragus of the auricle.

Glabella: Located on the soft tissue, the most prominent point in the midsagittal plane of the forehead.

Lateral nasal cartilage: A generally triangular plate of cartilage. Its superior margin is attached to the nasal bone and frontal process of the maxilla, and its inferior margin is connected to the greater alar cartilage.

Greater alar cartilage: A plate of cartilage lying below the lateral nasal cartilage. It is curved around the anterior part of the naris. Its posterior end is connected to the frontal process of the maxilla by a tough fibrous membrane containing three or four minor cartilages of the ala.

Nares (Nostrils): Approximately ellipsoidal apertures forming the entrance to the nasal cavity. The singular form of nares is naris (nostril). The nares are separated by the nasal septum.

Naso-labial sulcus or Naso-labial fold: The skin fold or groove that runs from each side of the nose to the corners of the mouth, separating the cheeks from the upper lip.

Naso-labial angle: The angle between the columella and the upper lip, while intersecting subnasale.

Otobasion inferior: The lowest point of attachment of the auricle to the skin of the face.

Otobasion superior: The highest point of attachment of the auricle to the skin of the face.

Pronasale: the most protruded point or tip of the nose, which can be identified in lateral view of the rest of the portion of the head.

Philtrum: the midline groove that runs from lower border of the nasal septum to the top of the lip in the upper lip region.

Pogonion: Located on the soft tissue, the most anterior midpoint of the chin.

Ridge (nasal): The nasal ridge is the midline prominence of the nose, extending from the Sellion to the Pronasale.

Sagittal plane: A vertical plane that passes from anterior (front) to posterior (rear). The midsagittal plane is a sagittal plane that divides the body into right and left halves.

Sellion: Located on the soft tissue, the most concave point overlying the area of the frontonasal suture.

Septal cartilage (nasal): The nasal septal cartilage forms part of the septum and divides the front part of the nasal cavity.

Subalare: The point at the lower margin of the alar base, where the alar base joins with the skin of the superior (upper) lip.

Subnasal point: Located on the soft tissue, the point at which the columella merges with the upper lip in the midsagittal plane.

Supramenton: The point of greatest concavity in the midline of the lower lip between labrale inferius and soft tissue pogonion

### 5.11.4.2 Anatomy of the Skull

Frontal bone: The frontal bone includes a large vertical portion, the squama frontalis, corresponding to the region known as the forehead.

Mandible: The mandible forms the lower jaw. The mental protuberance is the bony protuberance of the jaw that forms the chin.

Maxilla: The maxilla forms the upper jaw and is located above the mandible and below the orbits. The frontal process of the maxilla projects upwards by the side of the nose, and forms part of its lateral boundary.

Nasal bones: The nasal bones are two small oblong bones, varying in size and form in different individuals; they are placed side by side at the middle and upper part of the face, and form, by their junction, the “bridge” of the nose.

Nasion: The intersection of the frontal bone and the two nasal bones, a depressed area directly between the eyes and superior to the bridge of the nose.

Occipital bone: The occipital bone is situated at the back and lower part of the cranium. It includes an oval aperture, the foramen magnum, through which the cranial cavity communicates with the vertebral canal. The curved plate behind the foramen magnum is the squama occipitalis.

Orbit: The bony cavity in the skull to contain the eyeball.

Parietal bones: The parietal bones are the bones that, when joined together, form the roof and sides of the cranium.

Temporal bones: The temporal bones are situated on the bases and sides of the skull, and support that part of the face known as the temple.

Zygomatic bones: The face includes two zygomatic bones, located in the upper and lateral parts of the face and forming the prominence of the cheek.

### 5.11.4.3 Anatomy of the Respiratory System

Diaphragm: A sheet of muscle that extends across the bottom of the rib cage. The diaphragm separates the thoracic cavity, containing the heart, lungs and ribs, from the abdominal cavity. As the diaphragm contracts the volume of the thoracic cavity increases and air is drawn into the lungs.

Larynx: The larynx, or voice box houses the vocal folds and connects the inferior part of the pharynx (hypopharynx) with the trachea.

Lungs: The organs of respiration in humans. The conducting zone of the lungs contains the trachea, the bronchi, the bronchioles, and the terminal bronchioles. The respiratory zone contains the respiratory bronchioles, the alveolar ducts, and the alveoli.

Nasal cavity: The nasal cavity (or nasal fossa) is a large air filled space above and behind the nose in the middle of the face. The nasal cavity is divided in two by a vertical fin called the nasal septum. On the sides of the nasal cavity are three horizontal outgrowths called nasal conchae (singular “concha”) or turbinates. To the front of the nasal cavity is the nose, while the back blends, via the choanae, into the nasopharynx.

Pharynx: The part of the throat situated immediately inferior to (below) the nasal cavity, and superior to the oesophagus and larynx. The pharynx is conventionally divided into three sections: the nasopharynx (epipharynx) (the nasal part of the pharynx), the oropharynx (mesopharynx) (the oral part of the pharynx), and the laryngopharynx (hypopharynx).

### 5.11.5 Patient Interface

Anti-asphyxia valve (AAV): The component or sub-assembly of a mask system that, by opening to atmosphere in a failsafe manner, reduces the risk of excessive CO2 rebreathing by a patient.

Functional dead space: (description to be inserted here)

Headgear: Headgear will be taken to mean a form of positioning and stabilizing structure designed to hold a device, e.g. a mask, on a head.

Plenum chamber: a mask plenum chamber will be taken to mean a portion of a patient interface having walls at least partially enclosing a volume of space, the volume having air therein pressurised above atmospheric pressure in use. A shell may form part of the walls of a mask plenum chamber.

Vent: (noun): A structure that allows a flow of air from an interior of the mask, or conduit, to ambient air for clinically effective washout of exhaled gases. For example, a clinically effective washout may involve a flow rate of about 10 litres per minute to about 100 litres per minute, depending on the mask design and treatment pressure.

### 5.11.6 Shape of Structures

Products in accordance with the present technology may comprise one or more three-dimensional mechanical structures, for example a mask cushion or an impeller. The three-dimensional structures may be bounded by two-dimensional surfaces. These surfaces may be distinguished using a label to describe an associated surface orientation, location, function, or some other characteristic. For example a structure may comprise one or more of an anterior surface, a posterior surface, an interior surface and an exterior surface. In another example, a seal-forming structure may comprise a face-contacting (e.g. outer) surface, and a separate non-face-contacting (e.g. underside or inner) surface. In another example, a structure may comprise a first surface and a second surface.

To facilitate describing the shape of the three-dimensional structures and the surfaces, we first consider a cross-section through a surface of the structure at a point, p. See FIG. 3B to FIG. 3F, which illustrate examples of cross-sections at point p on a surface, and the resulting plane curves. FIGS. 3B to 3F also illustrate an outward normal vector at p. The outward normal vector at p points away from the surface. In some examples we describe the surface from the point of view of an imaginary small person standing upright on the surface.

### 5.11.6.1 Curvature in One Dimension

The curvature of a plane curve at p may be described as having a sign (e.g. positive, negative) and a magnitude (e.g. 1/radius of a circle that just touches the curve at p).

Positive curvature: If the curve at p turns towards the outward normal, the curvature at that point will be taken to be positive (if the imaginary small person leaves the point p they must walk uphill). See FIG. 3B (relatively large positive curvature compared to FIG. 3C) and FIG. 3C (relatively small positive curvature compared to FIG. 3B). Such curves are often referred to as concave.

Zero curvature: If the curve at p is a straight line, the curvature will be taken to be zero (if the imaginary small person leaves the point p, they can walk on a level, neither up nor down). See FIG. 3D.

Negative curvature: If the curve at p turns away from the outward normal, the curvature in that direction at that point will be taken to be negative (if the imaginary small person leaves the point p they must walk downhill). See FIG. 3E (relatively small negative curvature compared to FIG. 3F) and FIG. 3F (relatively large negative curvature compared to FIG. 3E). Such curves are often referred to as convex.

### 5.11.6.2 Curvature of Two Dimensional Surfaces

A description of the shape at a given point on a two-dimensional surface in accordance with the present technology may include multiple normal cross-sections. The multiple cross-sections may cut the surface in a plane that includes the outward normal (a “normal plane”), and each cross-section may be taken in a different direction. Each cross-section results in a plane curve with a corresponding curvature. The different curvatures at that point may have the same sign, or a different sign. Each of the curvatures at that point has a magnitude, e.g. relatively small. The plane curves in FIGS. 3B to 3F could be examples of such multiple cross-sections at a particular point.

Principal curvatures and directions: The directions of the normal planes where the curvature of the curve takes its maximum and minimum values are called the principal directions. In the examples of FIG. 3B to FIG. 3F, the maximum curvature occurs in FIG. 3B, and the minimum occurs in FIG. 3F, hence FIG. 3B and FIG. 3F are cross sections in the principal directions. The principal curvatures at p are the curvatures in the principal directions.

Region of a surface: A connected set of points on a surface. The set of points in a region may have similar characteristics, e.g. curvatures or signs.

Saddle region: A region where at each point, the principal curvatures have opposite signs, that is, one is positive, and the other is negative (depending on the direction to which the imaginary person turns, they may walk uphill or downhill).

Dome region: A region where at each point the principal curvatures have the same sign, e.g. both positive (a “concave dome”) or both negative (a “convex dome”).

Cylindrical region: A region where one principal curvature is zero (or, for example, zero within manufacturing tolerances) and the other principal curvature is non-zero.

Planar region: A region of a surface where both of the principal curvatures are zero (or, for example, zero within manufacturing tolerances).

Edge of a surface: A boundary or limit of a surface or region.

Path: In certain forms of the present technology, ‘path’ will be taken to mean a path in the mathematical—topological sense, e.g. a continuous space curve from f(0) to f(1) on a surface. In certain forms of the present technology, a ‘path’ may be described as a route or course, including e.g. a set of points on a surface. (The path for the imaginary person is where they walk on the surface, and is analogous to a garden path).

Path length: In certain forms of the present technology, ‘path length’ will be taken to mean the distance along the surface from f(0) to f(1), that is, the distance along the path on the surface. There may be more than one path between two points on a surface and such paths may have different path lengths. (The path length for the imaginary person would be the distance they have to walk on the surface along the path).

Straight-line distance: The straight-line distance is the distance between two points on a surface, but without regard to the surface. On planar regions, there would be a path on the surface having the same path length as the straight-line distance between two points on the surface. On non-planar surfaces, there may be no paths having the same path length as the straight-line distance between two points. (For the imaginary person, the straight-line distance would correspond to the distance ‘as the crow flies’.)

### 5.11.6.3 Space Curves

Space curves: Unlike a plane curve, a space curve does not necessarily lie in any particular plane. A space curve may be closed, that is, having no endpoints. A space curve may be considered to be a one-dimensional piece of three-dimensional space. An imaginary person walking on a strand of the DNA helix walks along a space curve. A typical human left ear comprises a helix, which is a left-hand helix, see FIG. 3Q. A typical human right ear comprises a helix, which is a right-hand helix, see FIG. 3R. FIG. 3S shows a right-hand helix. The edge of a structure, e.g. the edge of a membrane or impeller, may follow a space curve. In general, a space curve may be described by a curvature and a torsion at each point on the space curve. Torsion is a measure of how the curve turns out of a plane. Torsion has a sign and a magnitude. The torsion at a point on a space curve may be characterised with reference to the tangent, normal and binormal vectors at that point.

Tangent unit vector (or unit tangent vector): For each point on a curve, a vector at the point specifies a direction from that point, as well as a magnitude. A tangent unit vector is a unit vector pointing in the same direction as the curve at that point. If an imaginary person were flying along the curve and fell off her vehicle at a particular point, the direction of the tangent vector is the direction she would be travelling.

Unit normal vector: As the imaginary person moves along the curve, this tangent vector itself changes. The unit vector pointing in the same direction that the tangent vector is changing is called the unit principal normal vector. It is perpendicular to the tangent vector.

Binormal unit vector: The binormal unit vector is perpendicular to both the tangent vector and the principal normal vector. Its direction may be determined by a right-hand rule (see e.g. FIG. 3P), or alternatively by a left-hand rule (FIG. 3O).

Osculating plane: The plane containing the unit tangent vector and the unit principal normal vector. See FIGS. 30 and 3P.

Torsion of a space curve: The torsion at a point of a space curve is the magnitude of the rate of change of the binormal unit vector at that point. It measures how much the curve deviates from the osculating plane. A space curve which lies in a plane has zero torsion. A space curve which deviates a relatively small amount from the osculating plane will have a relatively small magnitude of torsion (e.g. a gently sloping helical path). A space curve which deviates a relatively large amount from the osculating plane will have a relatively large magnitude of torsion (e.g. a steeply sloping helical path). With reference to FIG. 3S, since T2>T1, the magnitude of the torsion near the top coils of the helix of FIG. 3S is greater than the magnitude of the torsion of the bottom coils of the helix of FIG. 3S

With reference to the right-hand rule of FIG. 3P, a space curve turning towards the direction of the right-hand binormal may be considered as having a right-hand positive torsion (e.g. a right-hand helix as shown in FIG. 3S). A space curve turning away from the direction of the right-hand binormal may be considered as having a right-hand negative torsion (e.g. a left-hand helix).

Equivalently, and with reference to a left-hand rule (see FIG. 3O), a space curve turning towards the direction of the left-hand binormal may be considered as having a left-hand positive torsion (e.g. a left-hand helix). Hence left-hand positive is equivalent to right-hand negative. See FIG. 3T.

### 5.11.6.4 Holes

A surface may have a one-dimensional hole, e.g. a hole bounded by a plane curve or by a space curve. Thin structures (e.g. a membrane) with a hole, may be described as having a one-dimensional hole. See for example the one dimensional hole in the surface of structure shown in FIG. 3I, bounded by a plane curve.

A structure may have a two-dimensional hole, e.g. a hole bounded by a surface. For example, an inflatable tyre has a two dimensional hole bounded by the interior surface of the tyre. In another example, a bladder with a cavity for air or gel could have a two-dimensional hole. See for example the cushion of FIG. 3L and the example cross-sections therethrough in FIG. 3M and FIG. 3N, with the interior surface bounding a two dimensional hole indicated. In a yet another example, a conduit may comprise a one-dimension hole (e.g. at its entrance or at its exit), and a two-dimension hole bounded by the inside surface of the conduit. See also the two dimensional hole through the structure shown in FIG. 3K, bounded by a surface as shown.

### 5.12 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Furthermore, “approximately”, “substantially”, “about”, or any similar term used herein means +/−5-10% of the recited value.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

1.-20. (canceled)

21. A patient interface for treating a patient with a respiratory disorder, comprising:
a respiratory pressure therapy (RPT) device including an electric blower configured to generate pressurized breathable air;
a user interface structure configured to engage against the patient's face, the user interface structure at least partially forming a plenum chamber configured to receive the pressurized breathable air;
a head-mounted interface configured to substantially cover the eyes of the patient;
a flow generator casing that at least partly encloses the head-mounted interface and the electric blower and is connected to the plenum chamber, the flow generator casing including at least one air opening to receive ambient air for delivery to the RPT device;
a positioning and stabilising structure configured to maintain the user interface structure in a therapeutically effective position,
wherein the user interface structure includes a stability cushion component and a therapy cushion component that is separate and distinct from the stability cushion component,
wherein the stability cushion component is configured to extend about a periphery of the patient's eyes and form a viewing opening to the head-mounted interface,
wherein the therapy cushion component is configured to form the plenum chamber and form a seal with the patient's nose and/or mouth, and
wherein the stability cushion component is configured to removably and interchangeably couple with the flow generator casing, and the therapy cushion component is configured to removably and interchangeably couple with the stability cushion component and/or the flow generator casing; and
a manifold configured to form an airflow path to convey the pressurized breathable air from the electric blower to the therapy cushion component,
wherein the manifold includes a first opening and a second opening that is oriented substantially perpendicular to the first opening such that the airflow path is configured to extend in an inferior direction through the first opening and into a posterior direction through the second opening, and
wherein the manifold includes a retainer part along the second opening configured to removably engage a retainer part provided to the therapy cushion component.

22. The patient interface according to claim 21, wherein the stability cushion component is configured to engage the patient's face to provide substantially complete sealing around the patient's eyes.

23. The patient interface according to claim 22, wherein the stability cushion component includes a chassis and a cushion configured to engage with the patient's face, and the chassis is configured and arranged to repeatably engage with and disengage from the flow generator casing.

24. The patient interface according to claim 23, wherein the chassis is relatively harder than the cushion.

25. The patient interface according to claim 23, wherein the cushion includes a face contacting portion configured to engage with the patient's face and a non-face contacting portion configured to connect the cushion to the chassis.

26. The patient interface according to claim 25, wherein the face contacting portion comprises a foam cushion including a face engaging surface configured to contact the patient's face around a periphery of the user's eyes.

27. The patient interface according to claim 25, wherein the face contacting portion comprises a silicone membrane including a face engaging surface configured to contact the patient's face around a periphery of the user's eyes.

28. The patient interface according to claim 25, wherein the non-face contacting portion includes a side wall provided to the chassis and a ledge configured to project away from the side wall in a cantilever manner, the non-face contacting portion configured to provide a resilient, spring-like support to the face contacting portion.

29. The patient interface according to claim 21, wherein the therapy cushion component is configured to removably and interchangeably couple with the flow generator casing.

30. The patient interface according to claim 21, wherein the therapy cushion component includes a front portion and a cushion configured to form a seal with the patient's nose and/or mouth.

31. The patient interface according to claim 30, wherein the front portion is relatively harder than the cushion.

32. The patient interface according to claim 30, wherein the cushion is a nasal cushion including a silicone membrane configured to form a seal with the patient's nose.

33. The patient interface according to claim 21, wherein the stability cushion component is provided in one size and/or type and the therapy cushion component is provided in multiple sizes and/or types.

34. The patient interface according to claim 21, wherein the manifold and the therapy cushion component cooperate to house an expiratory activated valve (EAV) to direct the pressurized breathable air from the blower to the therapy cushion component during inhalation and direct the pressurized breathable air from the therapy cushion component to an exhaust channel during exhalation.

35. The patient interface according to claim 21, wherein the blower includes at least a pair of impellers coupled to a common shaft and arranged in parallel.

36. The patient interface according to claim 21, further comprising a pair of batteries supported by the flow generator casing on respective sides of the blower.

37. The patient interface according to claim 21, wherein the positioning and stabilising structure is connected to at least a portion of the flow generator casing.

38. The patient interface according to claim 21, wherein the stability cushion component includes a chassis and a cushion configured to engage with the patient's face, wherein the chassis is configured and arranged to repeatably engage with and disengage from the flow generator casing, and wherein the therapy cushion component is configured to removably connect to the chassis of the stability cushion component such that the therapy cushion component and the stability cushion component form a unit that as a whole is removable and interchangeable with the flow generator casing.

39. The patient interface according to claim 21, wherein the manifold and the chassis of the stability cushion component form an integrally molded construction.
